SEQUENCE LISTING (28 sequences)

SEQ ID NO: 1 - protein, 28 aa, Homo sapiens
  MOD_RES (3)..(3): Amino acid in position 3 is modified with a fatty acid
GSSFLSPEHQRVQQRKESKKPPAKLQPR

SEQ ID NO: 2 - protein, 27 aa, Homo sapiens
  MOD_RES (3)..(3): Amino acid in position 3 is modified with a fatty acid
GSSFLSPEHQRVQRKESKKPPAKLQPR

SEQ ID NO: 3 - protein, 28 aa, Rattus rattus
  MOD_RES (3)..(3): Amino acid in position 3 is modified with a fatty acid
GSSFLSPEHQKAQQRKESKKPPAKLQPR

SEQ ID NO: 4 - protein, 25 aa, Homo sapiens
FLSPEHQRVQQRKESKKPPAKLQPR

SEQ ID NO: 5 - protein, 24 aa, Homo sapiens
FLSPEHQRVQQRKESKKPPAKLQP

SEQ ID NO: 6 - protein, 23 aa, Homo sapiens
FLSPEHQRVQQRKESKKPPAKLQ

SEQ ID NO: 7 - protein, 22 aa, Homo sapiens
FLSPEHQRVQQRKESKKPPAKL

SEQ ID NO: 8 - protein, 21 aa, Homo sapiens
FLSPEHQRVQQRKESKKPPAK

SEQ ID NO: 9 - protein, 20 aa, Homo sapiens
FLSPEHQRVQQRKESKKPPA

SEQ ID NO: 10 - protein, 19 aa, Homo sapiens
FLSPEHQRVQQRKESKKPP

SEQ ID NO: 11 - protein, 18 aa, Homo sapiens
FLSPEHQRVQQRKESKKP

SEQ ID NO: 12 - protein, 17 aa, Homo sapiens
FLSPEHQRVQQRKESKK

SEQ ID NO: 13 - protein, 16 aa, Homo sapiens
FLSPEHQRVQQRKESK

SEQ ID NO: 14 - protein, 15 aa, Homo sapiens
FLSPEHQRVQQRKES

SEQ ID NO: 15 - protein, 14 aa, Homo sapiens
FLSPEHQRVQQRKE

SEQ ID NO: 16 - protein, 13 aa, Homo sapiens
FLSPEHQRVQQRK

SEQ ID NO: 17 - protein, 12 aa, Homo sapiens
FLSPEHQRVQQR

SEQ ID NO: 18 - protein, 11 aa, Homo sapiens
FLSPEHQRVQQ

SEQ ID NO: 19 - protein, 10 aa, Homo sapiens
FLSPEHQRVQ

SEQ ID NO: 20 - protein, 9 aa, Homo sapiens
FLSPEHQRV

SEQ ID NO: 21 - protein, 8 aa, Homo sapiens
FLSPEHQR

SEQ ID NO: 22 - protein, 7 aa, Homo sapiens
FLSPEHQ

SEQ ID NO: 23 - protein, 6 aa, Homo sapiens
FLSPEH

SEQ ID NO: 24 - protein, 5 aa, Homo sapiens
FLSPE

SEQ ID NO: 25 - protein, 4 aa, Homo sapiens
FLSP

SEQ ID NO: 26 - protein, 3 aa, Homo sapiens
FLS

SEQ ID NO: 27 - protein, 2 aa, Homo sapiens
FL

SEQ ID NO: 28 - protein, 1 aa, Homo sapiens
F

(19) United States
(12) Patent Application Publication
(10) Pub. No.: US 2007/0037751 A1
(43) Pub. Date: Feb. 15, 2007

(54) Uses of secretagogues like ghrelin in cancer cachexia and for stimulating appetite

(75) Inventors: Birgitte Holst Lange, Gentofte (DK); Christian Hansen, Vedbaek (DK); Henrik Nilsson, Copenhagen K (DK)

Correspondence Address: BROWDY AND NEIMARK, P.L.L.C.;624 NINTH STREET, NW, SUITE 300, WASHINGTON, DC, 20001-5303, US

(73) Assignee: Gastrotech Pharma A/S, Copenhagen (DK)

(21) Appl. No.: 10/567,406

(22) PCT Filed: Aug. 6, 2004
(86) PCT No.: PCT/DK04/00529

§ 371 Date: Oct. 19, 2006

Related U.S. Application Data
(60) Provisional application No. 60/494,815, filed on Aug. 14, 2003.
(60) Provisional application No. 60/494,816, filed on Aug. 14, 2003.

(30) Foreign Application Priority Data
Aug. 6, 2003 (DK) PA 2003 01140
Aug. 6, 2003 (DK) PA 2003 01139
Sep. 5, 2003 (DK) PA 2003 01283
Oct. 24, 2003 (DK) PA 2003 01570
Oct. 24, 2003 (DK) PA 2003 01569
Apr. 7, 2004 (DK) PA 2004 00570

(51) Int. Cl. A61K 38/08; A61K 38/05; A61K 38/06

(52) U.S. Cl. 514/17; 514/18

(57) ABSTRACT

The present invention relates, in one aspect, to the use of a secretagogue compound for the preparation of a medicament for the prophylaxis or treatment of cancer cachexia in an individual in need of such treatment. In another aspect, the present invention relates to the use of a ghrelin-like compound for the preparation of a medicament for prophylaxis or treatment of cachexia in an individual by administering a subcutaneous dosage of said medicament to the individual. In a further aspect, the present invention relates to the use of a ghrelin-like compound or a pharmaceutically acceptable salt thereof for the preparation of a medicament for stimulation of appetite in an individual by administering a subcutaneous dosage of said medicament to the individual. Furthermore, the present invention relates to a number of new ghrelin-like compounds and uses thereof, as well as to pharmaceutical compositions and medical packaging comprising the new ghrelin-like compounds.

All patent and non-patent references cited in the application, or in the present application, are also hereby incorporated by reference in their entirety.

### FIELD OF INVENTION

In a first aspect, the present invention relates to the use of a secretagogue, such as a ghrelin-like compound, for the production of a medicament for the treatment or prevention of cachexia, stimulation of appetite, food intake and/or weight gain, as well as to a method of treating or preventing cachexia, stimulating appetite, food intake and/or weight gain in an individual in need thereof by administering a secretagogue, such as a ghrelin-like compound.

In another aspect, the present invention relates to the use of a secretagogue, such as a ghrelin-like compound, for the production of a medicament for the treatment or prevention of cancer cachexia as well as to a method of treating or preventing cancer cachexia in an individual in need thereof by administering a secretagogue, such as a ghrelin-like compound.

In another aspect, the present invention relates to the use of a secretagogue, such as a ghrelin-like compound, for the production of a medicament for the treatment or prevention of lipodystrophy as well as to a method of treating or preventing lipodystrophy in an individual in need thereof by administering a secretagogue, such as a ghrelin-like compound.

In another aspect, the invention relates to ghrelin-like compounds, pharmaceutical compositions comprising ghrelin-like compounds, and medical packagings comprising the pharmaceutical compositions.

### BACKGROUND OF INVENTION

Ghrelin is a bioactive peptide which originally was described to be involved in the control of GH secretion but later found to be a major regulator of appetite, food intake and energy homeostasis (1;2). Similar to many other bioactive peptides, ghrelin probably act both as a hormone, a paracrine substance and as a neurotransmitter.

The story of ghrelin, its receptor and synthetic compounds acting through this receptor unraveled in a unique “reverse” order. In the eighties a synthetic hexa-peptide from a series of opioid-like peptides was found to be able to release growth hormone (GH) from isolated pituitary cells (3). Since this action was independent of the growth hormone releasing hormone (GHRH) receptor, several pharmaceutical companies embarked upon drug discovery projects based on this hexa-peptide GH secretagogue (GHS) and its putative receptor. Several series of potent and efficient peptide as well as non-peptide GH secretagogues were consequently described in the mid nineties (4-6). However, it was only several years later that the receptor through which these artificial GH secretagogues acted was eventually cloned and shown to be a member of the 7TM G protein coupled receptor family (7;8). In 1999, the endogenous ligand for this receptor the hormone ghrelin was finally discovered (9). The main site for ghrelin production is the stomach, where the peptide is found in classical endocrine cells in the gastric mucosa.

From here, ghrelin is secreted in the pre-meal situation which results in a sharp, short-lived surge in plasma levels of ghrelin before the meal and starting 1-2 hours before and lasting a short while after initiation of the meal. Since ghrelin is the only peripherally produced orexigenic (appetite promoting) substance it is believed that the increase in plasma levels of ghrelin is crucial for the initiation of the meal.

In its role as a key initiator of appetite, ghrelin released from the endocrine cells in the mucosa of the GI tract may act both locally as a paracrine substance and centrally as a hormone, as discussed later in section relating to cancer cachexia.

Previously, ghrelin has been administered by continuous infusions for 270 minutes, which has shown that an increase in food intake can be obtained through intravenous administration of ghrelin. The doses were 5 pmol/kg/min (Wren et al JCEM 2001; 86(12)5992-5995). Recently, it was shown that infusion of ghrelin for 90 minutes could increase food intake by 30% in cancer cachexia patients. (Abstract P09 Digestive Hormones, Appetite and Energy Balance, Baylis and Starling meeting, London, June 2003).

These studies demonstrate that parenteral administration of ghrelin can increase appetite in both normal subjects and in patients with loss of appetite. However, a prolonged infusion regimen is clearly not an optimal administration form for both practical reasons and for physiological reasons.

### SUMMARY OF THE INVENTION

The present inventors have found that it is possible to obtain a sufficient effect of ghrelin when administered subcutaneously, in particular when administered subcutaneously prior to a meal, thereby ensuring a close mimic of the natural pre-meal situation.

Accordingly, the present invention relates to the use of a ghrelin-like compound for the preparation of a medicament for

a) prophylaxis or treatment of cachexia, and/or

b) stimulation of appetite,

in an individual by administering a subcutaneous dosage of said medicament to the individual,

wherein the ghrelin-like compound comprises a structure defined by formula I
Z¹-(X¹)ₘ—(X²)—(X³)ₙ-Z², wherein

Z¹ is an optionally present protecting group

each X¹ is independently selected from an amino acid, wherein said amino acid is selected from naturally occurring and synthetic amino acids,

X² is any amino acid selected from naturally occurring and synthetic occurring amino acids, said amino acid being modified with a bulky hydrophobic group, preferably an acyl group, or a fatty acid, each X³ is independently selected from an amino acid, wherein said amino acid is selected from naturally occurring and synthetic amino acids,

wherein one or more of X¹ and X³ optionally may be modified by a bulky hydrophobic group, preferably an acyl group, or a fatty acid,

Z² is an optionally present protecting group,

m is an integer in the range of from 1-10

n is 0 or an integer in the range of from 1-35.

In another aspect, the invention relates to the use of a secretagogue compound for the preparation of a medicament for

a) prophylaxis or treatment of cachexia, and/or

b) prophylaxis or treatment of lipodystrophy, and/or

c) stimulation of appetite, and/or

d) stimulation of food intake, and/or

e) stimulation of weight gain, and/or

f) increasing body fat mass,

including any combination of the above,

in an individual by administering a dosage of said medicament to the individual prior to or during a meal, said dosage comprising an amount of the secretagogue or a salt thereof equivalent to from 0.3 μg to 600 mg ghrelin. Preferably, said secretagogue is a ghrelin-like compound which comprises a structure defined by formula I, described above.

Preferred combinations are: a); b); c); d); e); and f) in isolation; as well as a)+b; a) +c); a)+d); a)+e); a)+f); b)+c); b)+d); b)+e); b)+f); a)+c)+d); a)+c)+e); a)+c)+f); a)+d)+e); a)+d)+f); a)+e)+f); a)+c)+d)+e); a)+c)+d)+f); a)+d)+e)+f); a)+c)+d)+e)+f); b)+c)+d); b)+c)+e); b)+c)+f); b)+d)+e); b)+d)+f); b)+e)+f); b)+c)+d)+e); b)+c)+d)+f); b)+d)+e)+f); and b)+c)+d)+e)+f).

The invention further relates to a method for the

a) prophylaxis or treatment of cachexia, and/or

b) stimulation of appetite.

in an individual by administering subcutaneously an effective dosage of a medicament comprising a secretagogue to said individual,

wherein the secretagogue is preferably a ghrelin-like compound, more preferably a ghrelin-like compound comprising a structure defined by formula 1, described above.

In yet another aspect the invention relates to a method for the

a) prophylaxis or treatment of cachexia, and/or

b) stimulation of appetite,

in an individual by administering an effective dosage of a medicament comprising a secretagogue to said individual prior to a meal,

wherein the secretagogue is preferably a ghrelin-like compound, more preferably a ghrelin-like compound which comprises a structure defined by formula I, described above.

In another aspect, the present invention further relates to the use of a secretagogue, in particular a ghrelin-like compound, including human ghrelin, in the treatment or prophylaxis of cancer cachexia, in particular, caused by the sub-types of cancer that induce a high degree of cachexia with an increase of REE, such as Lung cancer and Pancreatic cancer. In a preferred embodiment the invention relates to the use of a secretagogue, such as a ghrelin-like compound, such as a ghrelin-like compound comprising a structure defined by formula I, described above, for the preparation of a medicament for the treatment or prophylaxis of cancer cachexia

The present invention significantly lowers the risk of developing cancer cachexia, independent of the cause, such as independent of the particular therapeutic cause of, or therapeutic factor contributing to, the cancer cachexia.

In another aspect, the present invention relates to the use of a marker for monitoring the effect of the use of the ghrelin-like compound in accordance with the invention. Accordingly, in a preferred embodiment the invention relates to a method for monitoring the effect of the administration of the ghrelin-like compound of the invention, comprising measuring one or more markers, in particular markers selected from IGF-I, IGFBP-3, ALS (acidic labelled), thyroid hormones, sex hormones, and albumin, more preferably from IGF-I, IGFBP-3, ALS (acidic labelled), more preferably IGF-1. These markers are all low in cachetic patients and are expected to increase after treatment with ghrelin.

In another aspect, the present invention further relates to the use of a secretagogue, in particular a ghrelin-like compound, including human ghrelin, in the treatment or prophylaxis of lipodystrophy, whether acquired or congenital, local or generalized. In a preferred embodiment the invention relates to the use of a secretagogue, such as a ghrelin-like compound, such as a ghrelin-like compound comprising a structure defined by formula I (described above), for the preparation of a medicament for the treatment or prophylaxis of lipodystrophy. The present invention significantly lowers the risk of developing lipodystrophy, independent of the cause, such as independent of the particular therapeutic cause of, or therapeutic factor contributing to, the lipodystrophy. Hence, the present invention also extends to treatment of patients with HIV/AIDS by means of antiretroviral agents. For convenience, the term “antiretroviral therapy” is used herein to refer to the treatment of HIV/AIDS in patients by means of antiretroviral agents generally including combinations of such agents as is typically the case.

In a further aspect of the present invention, the invention relates to novel compounds relevant for the therapeutic and prophylactic indications herein. Accordingly, in another aspect the invention relates to a ghrelin-like compound wherein the ghrelin-like compound is defined by formula I
Z¹-(X¹)ₘ—(X²)—(X³)ₙ-Z², wherein

Z¹ is an optionally present protecting group

each X¹ is independently selected from an amino acid, wherein said amino acid is selected from naturally occurring and synthetic amino acids,

X² is any amino acid selected from naturally occurring and synthetic amino acids, said amino acid being modified with a acylgroup, wherein the acyl group is selected from the group of C7 acyl group, C9 acyl group, and C11 acyl group, such as from the group of C9 acyl group and Cl1 acyl group.

each X³ is independently selected from an amino acid, wherein said amino acid is selected from naturally occurring and synthetic amino acids,

Z² is an optionally present protecting group,

wherein one or more of X¹ and X³ optionally may be modified by a bulky hydrophobic group, preferably an acyl group, or a fatty acid,

m is 0 or an integer in the range of from 1-10

n is 0 or an integer in the range of from 1-35.

Furthermore, the invention relates to a pharmaceutical composition comprising a ghrelin-like compound as defined above or a pharmaceutically acceptable salt thereof and pharmaceutically acceptable carriers, vehicles and/or excipients as well a use of the compounds for the preparation of a medicament, in particular for

a) prophylaxis or treatment of cachexia, and/or

b) prophylaxis or treatment of lipodystrophy, and/or

c) stimulation of appetite, and/or

d) stimulation of food intake, and/or

e) stimulation of weight gain, and/or

f) increasing body fat mass,

including any combination of the above,

wherein preferred combinations are: a); b); c); d); e); and f) in isolation; as well as a)+b; a)+c); a)+d); a)+e); a)+f; b)+c); b)+d); b)+e); b)+f; a)+c)+d); a)+c)+e); a)+c)+f); a)+d)+e); a)+d)+f); a)+e)+f); a)+c)+d)+e); a)+c)+d) +f); a)+d)+e)+f; a)+c)+d)+e)+f); b)+c)+d); b)+c)+e); b)+c)+f); b)+d)+e); b)+d)+f); b)+e)+f); b)+c)+d)+e); b)+c)+d)+f); b)+d)+e)+f; and b)+c)+d)+e)+f.

In a preferred aspect of the invention the ghrelin-like compound is administered with a substance capable of increasing the half-life of the ghrelin-like compound, for example by incorporating the ghrelin-like compound into liposomes, micelles, iscoms, and/or microspheres or other transport molecules, in particular to protect the modified amino acid from being desacylated. Accordingly, the invention further relates to a pharmaceutical composition comprising the compound as defined above or a pharmaceutically acceptable salt thereof and pharmaceutically acceptable carriers, vehicles and/or excipients said composition further comprising transport molecules, such as liposomes, micelles, and/or microspheres.

In yet a further aspect the invention relates to a medical packaging comprising one or more dosage units of a pharmaceutical composition comprising a compound as defined above or a pharmaceutically acceptable salt thereof and pharmaceutically acceptable carriers, vehicles and/or excipients.

In a preferred embodiment the medical packaging comprises the pharmaceutical composition as defined above with predefined amounts of dosage units.

In one aspect the invention relates to a medical packaging comprising a pharmaceutical composition comprising a compound as defined above or a pharmaceutically acceptable salt thereof and pharmaceutically acceptable carriers, vehicles and/or excipients, said packaging having from one to three dosage units.

In another aspect the invention relates to a medical packaging comprising a pharmaceutical composition comprising a compound as defined above or a pharmaceutically acceptable salt thereof and pharmaceutically acceptable carriers, vehicles and/or excipients, said packaging having from 7 to 28 dosage units.

In all embodiments of the present invention, the medicament can be administered as a bolus injection or by fast running infusion, i.e. an infusion preferably lasting less than 120 minutes, such as less than 90 minutes, for example less than 60 minutes, such as less than 45 minutes, such as less than 30 minutes, for example less than 25 minutes, such as less than 20 minutes, such as less than 15 minutes, for example less than 12 minutes, such as less than 10 minutes, such as less than 8 minutes, for example less than 6 minutes, such as less than 5 minutes, such as less than 4 minutes, for example less than 3 minutes, such as less than 2 minutes, such as less than 1 minute.

A Y-formed catheter can be used for rapid infusion. A solution of the ghrelin-like compound can be injected through one catheter entry port and optionally saline can, if desirable, be injected through the other catheter entry port.

The bolus injection or the fast running infusion can be administered prior to a meal or during a meal as described in more detail herein below. In one preferred embodiment the medicament is administered as a bolus. The bolus is preferably administered subcutaneously.

### DETAILED DESCRIPTION OF THE INVENTION

Definitions

Affinity: the strength of binding between receptors and their ligands, for example between an antibody and its antigen.

Amino Acid Residue: An amino acid formed upon chemical digestion (hydrolysis) of a polypeptide at its peptide linkages. The amino acid residues described herein are preferably in the “L” isomeric form. However, the amino acid encompasses every amino acid such as L-amino acid, D-amino acid, alpha-amino acid, beta-amino acid, gamma-amino acid, natural amino acid and synthetic amino acid or the like as long as the desired functional property is retained by the polypeptide. NH₂ refers to the free amino group present at the amino terminus of a polypeptide. COOH refers to the free carboxy group present at the carboxy terminus of a polypeptide. In keeping with standard polypeptide, abbreviations for amino acid residues are shown in the following Table of Correspondence:

TABLE OF CORRESPONDENCE

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| Z | Glx | Glu and/or Gln |
| W | Trp | tryptophan |
| R | Arg | arginine |
| D | Asp | aspartic acid |
| N | Asn | asparagine |
| B | Asx | Asn and/or Asp |
| C | Cys | cysteine |
| X | Xaa | Unknown or other |

It should be noted that all amino acid residue sequences represented herein by formulae have a left-to-right orientation in the conventional direction of amino terminus to carboxy terminus. In addition, the phrase “amino acid residue” is broadly defined to include the amino acids listed in the Table of Correspondence and modified and non-naturally occurring amino acids. Furthermore, it should be noted that a dash at the beginning or end of an amino acid residue sequence indicates a peptide bond to a further sequence of one or more amino acid residues or a covalent bond to an amino-terminal group such as NH₂ or acetyl or to a carboxy-terminal group such as COOH.

Anti-neoplastic treatment: Treatment aimed at halting or reducing abnormal tissue growth (such as a neoplasm) in an individual. Examples of such treatment include cancer therapies, such as radiotherapy or chemotherapy.

Appetite: Appetite in an individual is assessed by measuring the amount of food ingested and by assessing the individual's desire to eat. Appetite (i.e., hunger) is typically assessed with a short questionnaire given to individuals on a random basis several times a week. Typically, subjects rate their hunger, preoccupation with food, and desire to eat greater quantities and different types of food by answering the questions using analogue scales ranging from 1, not at all, to 5, extremely.

BMI measures your height/weight ratio. It is determined by calculating weight in kilo-grams divided by the square of height in meters. The BMI“normal” range is 19-22.

Body fat mass: Body fat mass can be measured e.g. by the fat fold technique: In this technique, a pincer-type caliper is used to measure subcutaneous fat by determining skin fold thickness at representative sites on the body. These skin fold measurements are then used to compute body fat by either adding the scores from the various measurements and using this value as an indication of the relative degree of fatness among individuals or by using the measurements in mathematical equations that have been developed to predict percent body fat.

Concentration equivalent: A concentration equivalent is an Equivalents dosage being defined as the dosage of a ghrelin-like compound having in vitro and/or in vivo the same response as evaluated from a dosage-response curve of wild-type ghrelin.

Dissociation constant, Kd: a measure to describe the strength of binding (or affinity or avidity) between receptors and their ligands, for example an antibody and its antigen. The smaller Kd the stronger binding.

Fusion Polypeptide: A polypeptide comprised of at least two polypeptides and a linking sequence to operatively link the two polypeptides into one continuous polypeptide. The two polypeptides linked in a fusion polypeptide are typically derived from two independent sources, and therefore a fusion polypeptide comprises two linked polypeptides not normally found linked in nature.

Ghrelin: a polypeptide as described in Kojima M, Hosoda H, Date Y, Nakazato M, Matsuo H, Kangawa K 1999 Ghrelin is a growth-hormone-releasing acylated peptide from stomach. Nature 402:656-660. Human 28 aa ghrelin has the amino acid of SEQ ID NO: 1.

Ghrelin-like compound: the term “ghrelin-like compound” as used herein refers to any compound which mimicks the function of wild-type ghrelin, in particular wild-type human ghrelin, particularly in terms of the ghrelin functions leading to the desired therapeutic effects described herein, such as stimulation of appetite and/or treatment and/or prophylaxis of cachexia and is defined by the formula I:
Z¹-(X¹)ₘ—(X²)—(X³)ₙ-Z², wherein

Z¹ is an optionally present protecting group

each X¹ is independently selected from an amino acid, wherein said amino acid is selected from naturally occurring and synthetic amino acids,

X² is any amino acid selected from naturally occurring and synthetic occurring amino acids, said amino acid being modified with a bulky hydrophobic group, preferably an acyl group, or a fatty acid,

each X³ is independently selected from an amino acid, wherein said amino acid is selected from naturally occurring and synthetic amino acids,

wherein one or more of X¹ and X³ optionally may be modified by a bulky hydrophobic group, preferably an acyl group, or a fatty acid,

Z² is an optionally present protecting group,

m is an integer in the range of from 1-10

n is 0 or an integer in the range of from 1-35.

GHS: growth hormone secretagogue

GHS-R 1a: the receptor for GHS. GHS-R 1 a is also denoted GHS 1a.

Immunologically distinct: The phrase immunologically distinct refers to the ability to distinguish between two polypeptides on the ability of an antibody to specifically bind one of the polypeptides and not specifically bind the other polypeptide.

HAART: Highly active antiretroviral therapy.

Individual: A living animal or human susceptible to a condition, in particular a cachectic condition as defined herein. In preferred embodiments, the subject is a mammal, including humans and non-human mammals such as dogs, cats, pigs, cows, sheep, goats, horses, rats, and mice. In the most preferred embodiment, the subject is a human.

Isolated: is used to describe the various ghrelin-like compounds, polypeptides and nucleotides disclosed herein, that have been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. In preferred embodiments, the polypeptide will be purified.

Modified amino acid: an amino acid wherein an arbitrary group thereof is chemically modified. In particular, a modified amino acid chemically modified at the alpha-carbon atom in an alpha-amino acid is preferable.

Monoclonal Antibody: The phrase monoclonal antibody in its various grammatical forms refers to a population of antibody molecules that contains only one species of antibody combining site capable of immunoreacting with a particular antigen.

Non-acylated ghrelin-like compound: a ghrelin like-compound as defined herein, which does not contain an acyl group attached to any of its constitutent amino acids.

Palliative treatment: a treatment which relieves or sooths the symptoms of a disease or disorder but without effecting a cure.

Polyclonal antibody: Polyclonal antibodies are a mixture of antibody molecules recognising a specific given antigen, hence polyclonal antibodies may recognise different epitopes within said antigen.

Polypeptide: The phrase polypeptide refers to a molecule comprising amino acid residues which do not contain linkages other than amide linkages between adjacent amino acid residues.

Receptor: A receptor is a molecule, such as a protein, glycoprotein and the like, that can specifically (non-randomly) bind to another molecule.

REE: Resting energy expenditure.

Secretagogue: a growth hormone secretagogue, ie. a substance stimulating growth hormone release, such as ghrelin or a ghrelin-like compound. A secretagogue according to the invention may for example be selected from the group of:

L-692-429, L-692-585 (Benzoelactam compounds)

MK677 (Spiroindaner)

G-7203, G-7039, G-7502 (Isonipecotic acid peptidomimetic)

NN703, ipamorelin.

In particular the secretagogue is a ghrelin-like compound, including 28 aa human ghrelin. The secretagogue may in one embodiment be non-acylated, for instance a non-acylated form of ghrelin or a non-acylated ghrelin-like compound.

Surfactant molecule: Molecule comprising a hydrophobic part and a hydrophilic part, i.e. molecule capable of being present in the interphase between a lipophilic phase and a hydrophilic phase.

Indications

The present invention relates to the use of a secretagogue, such as a ghrelin-like compound, in the treatment or prophylaxis of conditions e.g. relating to pathological weight or fat loss, including

a) prophylaxis or treatment of cachexia, and/or

b) prophylaxis or treatment of lipodystrophy, and/or

c) stimulation of appetite, and/or

d) stimulation of food intake, and/or

e) stimulation of weight gain, and/or

f) increase of body fat mass.

In particular, the present invention relates to the treatment or prophylaxis of cachexia and/or the stimulation of appetite, most preferably the prophylaxis or treatment of cachexia.

Cachexia

The word cachexia comes from the Greek kakos for “bad” and hexis for “condition.”Cachexia is one of the most distressing and devastating symptoms of several severe diseases, such as cancer, robbing people of their energy, sense of well-being, and quality of life, and increasing their dependence on others. Cachexia often accompanies malignancies of the pancreas, stomach, esophagus, lung, and intestines.

The foremost sign of cachexia is weight loss, not only of fatty tissue but also of muscle tissue and even bone. This non-fatty tissue is also known as “lean body mass.”

In addition, there is loss of appetite (anorexia), weakness (asthenia), and a drop in hemoglobin level (anemia).

Treatment of cachexia is not simply a matter of eating more. Even if the person wants to eat, even if he or she tries to eat, even if the person is given nutrients through a stomach tube or intravenously, the condition will normally not be reversed.

Recent research has revealed that the condition is now regarded as part of the body's reaction to the presence of the underlying disease. Recent research also indicates that, in some cases, tumors themselves produce substances that induce cachexia.

Cachexia, or wasting, as it may also be called is seen with several diseases, such as AIDS, cancer, post hip fracture, chronic heart failure, chronic lung disease, such as COLD, COPD, liver cirrhosis, renal failure, and autoimmune diseases such as rheumatoid arthritis and systemic lupus, sepsis and severe infection. Furthermore, wasting is also seen in aging.

Cachexia is found as the terminal state of many different clinical conditions or in chronic diseases such as cancer, infections, AIDS, congestive heart failure, rheumatoid arthritis, tuberculosis, post-hip fracture, cystic fibrosis and Crohn's disease. It can also occur in elderly people who do not have any obvious symptoms of disease. Although cachexia represents the complex metabolic syndrome that is seen in such patients it is commonly recognized as a progressive weight loss with depletion of host reserves of adipose tissue and skeletal muscle.

Cancer Cachexia

The core of cancer cachexia syndrome relates to the problem of progressive tumor growth and the catabolic side effects of conventional anti-neoplastic therapy. These two phenomena give rise to alterations in the neuro-endocrine system, to the production of a variety of pro-inflammatory cytokines and to the release of cancer specific cachectic factors. In turn, these mediators cause either a reduction in food intake, abnormality in the metabolism or a combination of these two.

Cancer cachexia is reported to occur in about half of all cancer patients and is associated with more than 20 percent of cancer deaths (Tisdale M J. Nat Rev Cancer 2002, 2:862871). The condition often occurs during advanced cancer, in particular when metastatic tumours are present in the body. Cachexia is also more common in children and elderly patients

Specific cancers are also consistently identified where the frequency of cancer cachexia is particularly high:
Upper GI cancers (including: pancreas, stomach, oesophagus, and liver) (Bruera E. BMJ 1997;315:1219-1222.(8 November); (Palesty J A et al. Dig Dis 2003;21:198-213)
Lung cancer (www.oxandrin .com), in particular small cell lung cancer
Head and neck cancer (www.oxandrin.com)
Colorectal cancer
Other solid tumours (Bruera E. BMJ 1997;315:1219-1222.(8 November)

For example, the Oxandrin website (www.oxandrin.com) states that ‘In an Eastern Cooperative Oncology Group (ECOG) study addressing the impact of weight loss in advanced cancer, IWL (involuntary weight loss) was associated with an approximate 50% drop in survival and decreased tolerance of cancer therapy. Cancer sites associated with the greatest risks for weight loss are those affecting the aerodigestive tract (lung, head and neck, and esophagus) and the gastrointestinal system, especially pancreas, stomach, and liver.’

Furthermore, at the moment of diagnosis, 80% of all patients with cancer in upper GI tract and 60% of all patients with lung cancer have already experienced substantial weight loss (Bruera 1219-22). On average, the prevalence of cachexia increases from 50 percent to more than 80% percent before death and in more than 20% of the patients cachexia is the main cause of death (Bruera 1219-22).

Detection of cancer cachexia: The nutritional state is evaluated with a combination of clinical assessment, antropometric tests (body weight, skin fold thickness and mid arm circumference) and imaging (DEXA scan, MR scan, CT scan and bioelectric impedance meassuring). Cachexia is generally suspected if the involuntary weight loss of greater than 5% of the premorbid weight is observed within a six-month periode—especially when combined with muscle wasting.

The most commonly used laboratory parameter is serum albumin. It is however an unspecific parameter. Other markers are proteins with a short half life transferrrin and transthyretin has also been used.

Other markers of canchexia are IGF-1, IGFBP-3, ALP (alkaline phosphatase) and testosterone.

Relationship Between Cancer and Cancer Cachexia

Cancer may cause cachexia through a variety of mechanisms, including induction of anorexia and/or increase or change of metabolism, as described below:

Anorexia: Energy intake has been shown to be substantially reduced among weight-loosing cancer patients. Cancer patients may frequently suffer from physical obstruction of the GI tract, pain, depression, constipation, malabsorption, debility or the side effects of treatment such as opiates, radiotherapy or chemotherapy, which all may decrease food intake (Barber, Ross, and Fearon 133-41). Cancer associated hypercalcemia may also induce nausea, vomiting and appetite loss. However there remain a large number of patients with cancer in whom there is no obvious clinical cause of reduced food intake.

The central mechanism of cancer-induced anorexia and cachexia is complex and includes many different cytokines, hormones and other factors produced by the cancer cells.

Leptin: In normal physiological situations leptin plays an important role in triggering the adaptive response to starvation, since weight loss causes leptin levels to fall in proportion to the loss of body fat. However, in cancer patients an increased level of cytokines (IL-1, IL-6, TNF-α, INF-γ) produced by the cancer cells may stimulate the expression and/or the release of leptin. Another possible mechanism of the cytokines is that they mimic the hypothalamic effect of excessive negative feedback signalling from leptin, leading to the prevention of the normal compensatory mechanism regarding food intake and body weight.

NPY (Neuropeptide Y): The hypothalamic NPY system is one of the key neural pathways disrupted in anorexia induced by IL-1 or other cytokines. The cytokines decreases the sensitivity for NPY.

Melanocortins: Abberant melanocortin signalling may be a contributing factor in both anorexia and cachexia. Despite marked loss of body weight which would normally be expected to down-regulate the anorexigenic melanocortin signaling system as a way to conserve energy stores, the melanocortin system remains active during cancer induced cachexia. Central malanocortin blockade by AgRP or other antagonists reversed anorexia and cachexia in the animal models suggesting a pathogenic role of this system (Wisse, Schwartz, and Cummings 275-81).

Metabolism: Hyper metabolism is defined as an elevation of the resting energy expenditure (REE) and is a cardinal feature of cachexia. Total energy expenditure involves REE (app. 70%) and voluntary energy expenditure (app. 25%) and energy expenditure in digestion (5%). Voluntary energy expenditure may be decreased in cachexia which may manifest clinically as apathy, fatigue and depression.

The orexigenic and the anorexigenic signals are known to respectively decrease and increase sympatheic nervous activity, which regulate REE by activating thermogenesis in brown adipose tissue in rodents and possibly in muscle in humans, through induction of the mitochondrial uncoupling proteins (UCP). It has been suggested that activation of UCP in muscle and in white adipose tissue by cytokines might be one of the molecular mechanisms underlying the increase in the heat production and muscle wasting (Inui 72-91;Fearon and Moses 73-81).

Altered nutrition metabolism has also been described in patients with cancer. Solid tumors produce large amounts of lactate, which is converted back into glucose through a process that uses large amounts of ATP and is very energy inefficient thus further increasing the energy expenditure. Furthermore, tumor derived lipid mobilizing factor (LMF) has been shown to directly on adipocytes and cause increased lipolysis, leading to release of free fatty acid and glycerol (ref: Islam-Ali et al. Br J Cancer. Sep. 1, 2001 ;85(5):758-63). It has also been suggested that the increased level of cytokines may induce muscle protein catabolism indirectly by affecting the muscle repair processes.

The Rationale for Using Secretagogues in the Treatment of Cancer Cachexia

Without being bound by theory, the rationale for the treatment with a secretagogue, in particular a ghrelin-like compound is based on the following:

Ghrelin released from the endocrine cells in the mucosa of the GI tract may act both locally as a paracrine substance and centrally as a hormone. Locally, ghrelin may act as an initiator of afferent activity in for example afferent vagal neurons. Such neurons will relay the ghrelin stimulus to centers in the CNS such as the nucleus tractus solitarirus (NTS) which further communicate with appetite and energy homeostasis regulatory centers such as the paraventricular nucleus and arcuate nucleus in the hypothalamus. As a hormone, ghrelin is believed to act on central appetite regulating POMC and NPY/AGRP neurons, which express ghrelin receptors. Most of these neurones in the arcuate nucleus as such are located inside the blood brain barrier and is consequently not accessible to blood born messengers such as ghrelin. However, some POMC and NPY/AGRP neurons are found in the nearby median eminence, a cicumventricular organ, which is clearly outside the blood brain barrier and are therefore target for hormonally transmitted ghrelin signaling from the GI tract. However, recently it has been described that ghrelin is transported across the blood brain barrier (Banks et al. 822-27). It is important to note that at the central appetite regulatory center, for example at the NPY/AGRP neurons—i.e. the first level neurons in the stimulatory branch of appetite control—ghrelin acting through stimulatory ghrelin receptors is the only stimulatory input known from the periphery. All other hormones and neurotransmitters: leptin, insulin, PYY3-36, a-MSH etc. act as inhibitors on the NPY/AGRP neurons in this important “appetite gate-keeping”center. Since the NPY system in down-regulated during cancer induced cachexia ghrelin stimulation of this system may be able to normalize the condition. Similarly the melanocortin that is active during cancer induced cachexia, may be inhibited by Ghrelin through stimulation of AgRP.

Increase in ghrelin has also been shown to increase ATCH with a resulting increase in cortisol level. This action may have important beneficial implications for the treatment of cachexia, as cortisol decreases the level of cytokines (IL-1β, IL-6, TNF-α, IFN-α). Administration of glucocorticoids is already widely used in the palliative setting for symptoms associated with cancer (Inui 72-91)

Furthermore, it is known that ICV injection of ghrelin has been shown to decrease core body temperature in rodents, which indicates a decrease in the REE (Lawrence C B, Endocrinology. January 2002; 143(1):155-62). Again without being bound by theory it is expected that ghrelin will revert the increase in REE which is an important feature of the cachexia as described above.

The secretogogue, in particular a ghrelin-like compound, may be administered using any suitable regimen taking into account the knowledge of the expected cancer progress as well as the anti-neoplastic therapy regime.

In one embodiment, it is envisaged that, according to the present invention, a secretagogue can be administered to any individual suffering from any cancer type, regardless of etiology, to successfully treat, reduce or prevent cancer cachexia. Thus, in one preferred embodiment of the present invention, the treatment of an individual with a secretagogue, such as ghrelin or a ghrelin-like compound, is for the treatment or prevention of cancer cachexia caused by one or more of the following cancer types:
Acute Lymphoblastic Leukemia
Acute Myeloid Leukemia
Adrenocortical Carcinoma
AIDS-Related Cancers
AIDS-Related Lymphoma
Anal Cancer
Astrocytoma, Childhood Cerebellar
Astrocytoma, Childhood Cerebral
Basal Cell Carcinoma
Extrahepatic Bile Duct Cancer
Bladder Cancer
Bone Cancer, Osteosarcoma/Malignant Fibrous Histiocytoma
Brain Stem Glioma
Brain Tumor
Breast Cancer
Breast Cancer, Male
Bronchial Adenomas/Carcinoids
Burkitt's Lymphoma
Carcinoid Tumor
Carcinoma of Unknown Primary
Central Nervous System Lymphoma, Primary
Cerebral Astrocytoma/Malignant Glioma
Cervical Cancer
Childhood Cancers
Chronic Lymphocytic Leukemia
Chronic Myelogenous Leukemia
Chronic Myeloproliferative Disorders
Colon Cancer
Cutaneous T-Cell Lymphoma
Endometrial Cancer
Ependymoma, Childhood
Esophageal Cancer
Ewing's Family of Tumors
Extracranial Germ Cell Tumor, Childhood
Extragonadal Germ Cell Tumor
Eye Cancer, Intraocular Melanoma
Eye Cancer, Retinoblastoma
Gallbladder Cancer
Gastric (Stomach) Cancer
Gastrointestinal Carcinoid Tumor
Gestational Trophoblastic Tumor
Glioma
Hairy Cell Leukemia
Head and Neck Cancer
Hepatocellular (Liver) Cancer
Hodgkin's Lymphoma
Hypopharyngeal Cancer
Hypothalamic and Visual Pathway Glioma, Childhood
Intraocular Melanoma
Islet Cell Carcinoma (Endocrine Pancreas)
Kaposi's Sarcoma
Kidney (Renal Cell) Cancer
Laryngeal Cancer
Lip and Oral Cavity Cancer
Lung Cancer, Non-Small Cell
Lung Cancer, Small Cell
Lymphoma, AIDS-Related
Lymphoma, Burkitt's
Lymphoma, Cutaneous T-Cell
Lymphoma, Non-Hodgkin's
Macroglobulinemia, Waldenström's
Malignant Fibrous Histiocytoma of Bone/Osteosarcoma
Medulloblastoma, Childhood
Melanoma
Merkel Cell Carcinoma
Mesothelioma, Adult Malignant
Mesothelioma, Childhood
Metastatic Squamous Neck Cancer with Occult Primary
Multiple Endocrine Neoplasia Syndrome, Childhood
Multiple Myeloma/Plasma Cell Neoplasm
Mycosis Fungoides
Myelodysplastic Syndromes
Myelodysplastic/Myeloproliferative Diseases
Myeloma, Multiple
Chronic myeloproliferative disorders
Nasal Cavity and Paranasal Sinus Cancer
Nasopharyngeal Cancer
Nasopharyngeal Cancer, Childhood
Neuroblastoma
Oropharyngeal Cancer
Osteosarcoma/Malignant Fibrous Histiocytoma of Bone
Ovarian Cancer, Childhood
Ovarian Epithelial Cancer
Ovarian Germ Cell Tumor
Ovarian Low Malignant Potential Tumor
Pancreatic Cancer
Pancreatic Cancer
Paranasal Sinus and Nasal Cavity Cancer
Parathyroid Cancer
Penile Cancer
Pheochromocytoma
Pineoblastoma and Supratentorial Primitive Neuroectodermal Tumors, Childhood
Pituitary Tumor
Pleuropulmonary Blastoma
Prostate Cancer
Renal Pelvis and Ureter, Transitional Cell Cancer
Retinoblastoma
Rhabdomyosarcoma, Childhood
Salivary Gland Cancer
Adult-onset soft tissue Sarcoma
Sarcoma, Soft Tissue, Childhood
uterine Sarcoma
Sezary Syndrome
Skin Cancer (non-Melanoma)
Skin Carcinoma, Merkel Cell
Small Intestine Cancer
Supratentorial Primitive Neuroectodermal Tumors, Childhood
Cutaneous T-Cell Lymphoma
Testicular Cancer
Thymoma and Thymic Carcinoma
Thyroid Cancer
Transitional Cell Cancer of the Renal Pelvis and Ureter
Trophoblastic Tumor, Gestational
Ureter and Renal Pelvis, Transitional Cell Cancer
Urethral Cancer
Endometrial uterine cancer
Uterine Sarcoma
Vaginal Cancer
Visual Pathway and Hypothalamic Glioma, Childhood
Waldenström's Macroglobulinemia
Wilms' Tumor

As discussed above cancer cachexia may be due to a catabolic disorder, i.e. a hypermetabolic state as described above, either resulting from the progressive tumor growth or from the catabolic side effects of the anti-cancer therapy. However, the cancer cachexia may also be due to an anorectic disorder, such as is the case when the individual suffering from the cancer has no appetite or the position of the tumor reduces food intake.

Accordingly, in one embodiment of the invention the treatment with a secretagogue, such as a ghrelin-like compound, is for the treatment or prevention of cancer cachexia caused by a catabolic disorder. This is particularly suitable when the cancer is a GI tract cancers, especially upper GI tract cancers (it is to be understood herein that the term “upper GI tract cancer” also encompasses pancreatic cancer)”, lung cancer, in particular small cell lung cancer, liver cancer (it is to be understood herein that the term “liver cancer” also encompasses metastatic cancer processes in the liver).

In another embodiment of the invention the treatment with a secretagogue, such as a ghrelin-like compound, is for the treatment or prevention of cancer cachexia caused by an anorectic disorder.

In yet another embodiment the treatment with a secretagogue, such as a ghrelin-like compound, is for the treatment or prevention of cancer cachexia independent of how the cancer has induced the cachexia, as well as for cachexia caused by a combination of the catabolic disorder and the anorectic disorder.

In another preferred embodiment of the present invention, the treatment with a secretagogue, such as ghrelin or a ghrelin-like compound, is for the treatment or prevention of cancer cachexia caused by a solid tumour.

Another sub-group of cancers are those with anorexia caused by dysregulation of the central appetite regulatory centre in hypothalamus, where other possible reasons to eat less are excluded. In particular individuals in terminal cancer states where further cancer treatment is impossible would benefit from ghrelin treatment as a palliative treatment to increase food intake, improve the digestion and metabolism.

Accordingly, a third aspect of the invention relates to the palliative treatment of terminal cancer states in an individual in need thereof, such as wherein said individual is suffering from advanced-stage cancer, preferably terminal cancer.

In accordance with the above, the invention is particularly suitable for treating or preventing cachexia in an individual suffering from the following aerodigestive tract cancer forms:
Pancreatic cancer
cancer of the upper GI tract, such as stomach cancer and/or esophagus cancer.
head and neck cancer, in particular cancer of the thyroid or cancer of the salivary glands.
lung cancer, in particular small lung cell cancer.

In another preferred embodiment of the present invention, the treatment with a secretagogue, such as ghrelin or a ghrelin-like compound, is for the treatment or prevention of cancer cachexia caused by lower GI cancer, such as colorectal cancers, in particular by colon cancer.

In another preferred embodiment of the present invention, the treatment with a secretagogue, such as ghrelin or a ghrelin-like compound, is for the treatment or prevention of cancer cachexia caused by an endocrine cancer, i.e. a cancer in an endocrine organ of an individual's body.

The invention is also useful for treating individuals suffering from the following cancer forms:
cancer of the ovaries
breast cancer.

In a further preferred embodiment of the present invention, the treatment with a secretagogue, such as ghrelin or a ghrelin-like compound, is for the treatment or prevention of cancer cachexia caused in whole or in part by anti-cancer treatment, such as chemotherapy or radiotherapy or combinations thereof.

In one preferred embodiment of the present invention, the individual treated for cancer cachexia is elderly, such as 60-120 years old, more preferably 70-120 years old, such as 80-120 years old, for instance 90-120 years old. Equally preferably, said individual is a child, such as from 0-20 years old, for example 0-15 years old, such as 0-10 years old, for example 0-5 years old, such as 0-1 years old, such as a newborn child less than 2 months old.

In one embodiment it is preferred that the secretogogue, such as a ghrelin-like compound, is administered prophylactically for preventing a cachectic state. In this embodiment the treatment may be started before any anti-neoplastic treatment initiates. It may be administered continuously during the anti-neoplastic treatment or it may be administered at intervals, for example between periods with anti-neoplastic therapy. By administering during and in particular between the periods of anti-neoplastic therapy, the risk that the treated individual acquires infections and other complications may be reduced due to the better health conditions.

Treatment of cancer cachexia using a secretagogue, such as ghrelin or a ghrelin-like compound, may be achieved using any administration method known in the art. Preferably, it may be achieved using any of the administration methods described herein, more preferably using i.v. or subcutaneous administration, most preferably using subcutaneous administration methods.

Lipodystrophy

In another aspect, the invention relates to use of a secretagogue, such as ghrelin or a ghrelin-like compound as defined above, for the treatment of a lipodystrophic syndrome, or for the manufacture of a medicament for the treatment of a lipodystrophic syndrome. Lipodystrophic syndromes encompass a heterogeneous group of rare disorders characterized by partial or generalized loss of adipose tissue depots [Am J Med 2000 108, 143-152]. There are several different types of lipodystrophies and the degree of fat loss may vary from very small depressed areas to near complete absence of adipose tissue. Some patients may have only cosmetic problems while others may also have severe metabolic complications such as dyslipidemia, hepatic steatosis, and severe insulin resistance [Trend Endo Meta 200011:410-416]. These disorders can either be inherited (familial or genetic lipodystrophies) or can occur secondary to various types of illnesses or drugs (acquired lipodystrophies).

Inherited lipodystrophies are caused by mutations (alterations or blips) in a gene. Several genes responsible for different types of inherited lipodystrophies have been identified. These include AGPAT2 (1-acylglycerol-3-phosphate-O-acyltransferase 2), BSCL2 (Berardinelli-Seip congenital lipodystrophy 2) in Congenital Generalized Lipodystrophy (CGL), Lamin A/C (LMNA) gene in Familial Partial Lipodystrophy Dunnigan variety (Familial partial lipodystrophy), and PPARG (peroxisome proliferator-activated receptor gamma) gene in familial partial lipodystrophy. Several other candidate genes are currently under investigation for other varieties of inherited lipodystrophies.

Acquired lipodystrophies are; HMRT/HIV-induced Lipodystrophy in HIV-infected patients (LD-HIV), Acquired Generalized Lipodystrophy (AGL), Acquired Parital Lipodystrophy (APL) and localized lipodystrophy. Acquired lipodystrophies do not have a direct genetic basis. Rather, many mechanisms may be involved. One such mechanism may be an autoimmune response that destroys normal fat cells.

HAART/HIV-induced Lipodystrophy has become the most common aquired form of generalized Lipodystrophy. The overall incidence of these physical abnormalities is about 50% after 12-18 month of therapy with protease inhibitors. The difference between the present reports range from 18% to 83% percent due to confounding factors such as type and duration of the retroviral therapy. It has been suggested that the lipodystrophy syndrome associated with protease inhibitors may be due to partial analogy between lipid and adipocyte regulatory proteins and the catalytic site of HIV-1 protease to which the protease inhibitors bind (Carr et al lancet 1998, 351 1881-83).

Localized lipodystrophies are defined as a localized loss of subcutaneous fat from small areas or from parts of a limb. There may be single or multiple lesions. It is characterized by depressed areas corresponding to the loss of subcutaneous fat. In some cases, it may be associated with tender, painful nodules in the skin. Usually, it occurs in diabetic patients at the site of insulin injections. In some patients, fat loss occurs from areas where pressure is applied frequently. For example, pressing thigh against a make-up table.

The pathogenesis of lipodystrophy is largely unknown. However, accumulating evidence points at mitochondrial defect as central for an increased induction of apoptosis in the adipocytes. Several proteins encoded by HIV-1 trigger apoptosis by inducing permeabilization of the mitochondrial membrane. Several nucleoside analogs used clinically in the treatment of HIV-1 inhibit the replication of mitochondrial DNA (mtDNA) and/or increase the frequency of mtDNA mutations. Both of these factors may cause severe mitochondriopathy and contribute to lipodystrophy. A treatment that could inhibit the apoptosis of the adipocytes could be a very useful treatment of and especially a prevention for the development of lipodystrophy—in-particular in the HIV/HAARD induced from.

The metabolic consequences of lipodystrophy are highly important for the general health and the survival. The fact that insulin resistance and the consequent progression to diabetes can result from either obesity or lipodystrophy reflects the crucial role of adipose tissue in carbohydrate and lipid metabolism. In the absence of adequate adipocyte capacity, excess calories cannot be diverted to their normal storage depot; instead they accumulate as increased triglyceride stores in liver, in skeletal and cardiac muscle, and in the pancreatic R cell. This extra-adipose lipid accumulations, through as-yet unclear means, is associated with impaired insulin action and, often, diabetes.

In addition to their passive role as storage depots, normal adipocytes secrete a number of peptides (“adipokines”) that may influence insulin sensitivity and/or energy balance (Kahn J C I and TEM 2002). These include potential insulin sensitizers, such as leptin and Acrp30 (also known as adiponectin), and insulin antagonists, including TNF-α, IL-6, and possibly resistin. The insulin resistance of lipodystrophy may therefore be the result of disturbed lipid fluxes and/or abnormalities of adipokine secretion.

Therapy with rhGH has been reported to cause reduction in the size of ‘buffalo hump’ and truncal fat in a small number of patients. However, fat loss and lipid abnormalities did not improve and blood glucose control worsened.

Treatment of lipodystrophy using a secretagogue, such as ghrelin or a ghrelin-like compound, may be achieved using any administration method known in the art. Preferably, it may be achieved using any of the administration methods described herein, more preferably using i.v. or subcutaneous administration, most preferably using subcutaneous administration methods.

Quality of Life

In all embodiments of the present invention, it is preferred that the treatment method and/or pharmaceutical compositions and/or compounds of the present invention are capable of affording the individual thus treated an improved quality of life (QOL), for example as is caused by improved appetite and/or body weight and/or nutritional status. Thus, in one aspect the invention relates to improvements of Quality of Life using a secretagogue, such as ghrelin or a ghrelin-like compound as defined above. In another embodiment, said improvement in an individual's life quality is assessed using a “Quality of life” questionnaire, as is known to one skilled in the art.

Two validated quality of life surveys preferred for use in assessing improved quality of life as caused by the administration of the compounds of the present invention are as follows:

(i) Medical Outcomes Study Short-Form Health Survey (SF-36). The SF-36 contains 36 questions that assess eight aspects of the patients QOL; physical functioning (PF), role-physical functioning (RP), bodily pain (BP), general health (GH), vitality (VT), social functioning (SF), role emotional functioning (RE), and mental health (MH). According to the manual and interpretation guide responses to questions within scales are summed and linearly transformed to scale scores that range from 0, representing poor health status, to 100, representing optimal health status. The Swedish version has been validated and normative data have been presented for the general Swedish population (Sullivan M K J, Ware J. Hälsoenkät: svensk manual och tolkningsguide (SF-36 Health Survey. Swedish manual and interpretation guide). Göteborg: Sahigrenska University Hospital; 1994.)

(ii) EORTC QLQ-C30 (+3) questionnaire. The EORTC QLQ-C30 (version 1.0) is a 30 item core questionnaire intended for assessment of QOL among patients, the instrument is developed by the EORTC Quality of Life Study group. The first version has been validated in cancer patients and reference data from general populations have been published. The questionnaire comprises five functional scales; physical functioning ( five questions), role functioning (two questions), emotional functioning (four questions), cognitive functioning (two questions) and social functioning (two questions). There are three symptom scales; fatigue (three questions), nausea and vomiting (two questions) and pain (two questions), and there are six single items on dyspnoea, insomnia, loss of appetite, constipation, diarrhea and financial difficulties. Two global questions are asking about the patient's health status and overall QOL. All scales and single-items measures range in score from 0 to 100. A high score for the functioning scales and the global health status and QOL represents a high level of functioning/health status and QOL. A high score for the symptom/item scales represents a high level of symptoms/problems. The QOL scores can be calculated according to the EORTC QLQ-C 30 scoring manual.

Preferred questionnaires for assessing a patient's improved quality of life after treatment with one or more secretagogue compounds are given in Examples 8A, 8B and 8C.

In preferred embodiments of the present invention, treatment of patients with the described conditions results in a significant improvement in the patients quality of life. Preferably, the treatment results in a significant increase in quality of life as measured using any method for testing the quality of life including, but not limited to, the above mentioned questionnaires, e.g. an increase in the quality of life score(s), or a composite quality of life score, as appropriate for the individual measuring tool, or a decrease in score(s) related to the symptoms and/or problems, respectively. This increase or decrease, respectively, is preferably 1% above the score obtained prior to initiation of the treatment, more preferably 2% above, even more preferred 5%, such as 10%, even more preferred 20%, 50% or 75% above the pre-treatment score. In another embodiment, the treatment results in measurable increases in quality of life score such that the score after treatment is equal to the average score found in a comparable healthy subject pool, or close to such a “normal” score, i.e. more than 50% of the score, even more preferably 60% of the score, or more preferably 75% of the score. Further, in another embodiment, the treatment results in a decrease in the score(s) related to the symptoms and/or problems of at least 1%, more preferably 3%, even more preferably 5% or more preferred 10%, 20%, 30% or 50% of the score(s) prior to initiation of treatment. These increases or reductions, respectively, may refer to one, several, or all of the aspects of the individual quality of life measuring tool, or a composite score when appropriate.

Stimulation of Appetite, Food Intake, Weight Gain, Increase of Body Fat Mass

As written above, facilitating a weight gain or facilitating maintenance of weight, in particular in individuals suffering from a pathologically weight loss, is not only a matter of stimulating appetite and/or food intake but rather correcting the imbalance between energy intake and energy consumption, i.e. total body metabolism.

However, some individuals will still benefit from stimulation of appetite, particularly those individuals for whom a pathological process has led to a lowered appetite, which will naturally lead to an unhealthy weight loss. Thus, in one aspect the present invention relates to the stimulation of appetite by administering a secretagogue, such as a ghrelin-like compound. The stimulation of appetite may be measured using for instance a visual analog scale for measuring appetite, feeling of hunger or satiety level as described in example 8C. In a preferred embodiment of the invention, the stimulation is at least 5% compared to prior to the treatment, such as 10% higher, more preferably 20% higher or even more preferably 30%, 40% or 50% higher.

Stimulation of appetite, does not necessarily lead to an increase in food intake, and accordingly, the present invention further relates to another aspect: the stimulation of food intake by administering a secretagogue, such as a ghrelin-like compound. The food intake can be measured using a multitude of techniques including self-reporting using e.g. diaries or questionnaires, measurements of calorie-intake from a buffet meal, using weighing of food prior to ingestion, or weighing and analysis of paired quantities of food. The food intake may be measured on a meal basis, a daily basis, a weekly basis or a monthly basis. In a preferred embodiment of the invention, the treatment results in a 1% increase in food intake, such as an increase of 2%, more preferably 3%, or 5% or 7%, and even more preferred 10% above average food intake prior to initiation of treatment. In another embodiment, the treatment leads to increase in calorie intake irrespective of changes in food intake, since amount of food ingested may not be directly related to the ingested calorie intake, as the various food items such as fat, carbohydrates and proteins, contain different amounts of calories per amount food. In a preferred embodiment of the invention, the treatment results in a 1% increase in calorie intake, such as an increase of 2%, more preferably 3%, or 5% or 7%, and even more preferred 10% in calorie intake.

In a third aspect the present invention relates to stimulation of weight gain or maintaining a stable body-weight by administering a ghrelin-like compound.

Preferably the secretagogue, such as a ghrelin-like compound, is useful for stimulating food intake and weight gain, more preferably the secretagogue, such as a ghrelin-like compound, is useful for stimulating weight gain or for maintaining stable body weight.

As discussed below it is preferred that the secretagogue, such as a ghrelin-like compound, is administered prior to a meal, such as within 180 minutes prior to a meal, such as within 150 minutes prior to a meal, for example within 120 minutes prior to a meal, such as within 100 minutes prior to a meal, for example within 80 minutes prior to a meal, such as within 60 minutes prior to a meal, for example within 45 minutes prior to a meal, such as within 30 minutes prior to a meal, for example within 15 minutes prior to a meal. Furthermore, it is preferred that the ghrelin-like compound is administered subcutaneously.

Furthermore, a secretagogue, such as a ghrelin-like compound may be administered to facilitate maintenance of physical functioning, and/or facilitate recovery of physical function, for example in individuals recovering from major surgeries, such as insertion of a hip prosthesis, amputations, and bone fractures.

In particular the present invention is useful for treatment of under weight subjects, or for preventing loss of weight to a stage of under weight. Under weight subjects include those having a body weight about 3%, 5% or less, 10% or less, 20% or less, or 30% or less, than the lower end of “normal” weight range or Body Mass Index (“BMI”).“Normal” weight ranges are well known in the art and take into account factors such as a patient age, height, and body type. Furthermore, the invention is suitable for treating patients who have experienced an involuntary weight-loss prior to commencement of treatment, such as a weight-loss of 1% per month, 2% or less per month, or 5% or less per 6 months.

Increasing weight or appetite can be useful for a patient having a disease or undergoing treatment that affects weight or appetite. In addition, for example, farm animals such as pigs, cows and chickens can be treated to gain weight.

An increase in the body fat mass of an individual can be readily assessed by the skilled person using a number of state of the art techniques. In one embodiment the invention relates to an increase in body fat mass without the individual gaining weight overall. A preferred embodiment of the invention leads to an increase in body fat of 2% compared to prior to the initiation of treatment, more preferably 4%, such as 5%, and 8% and 10%, even more preferably 20% or 40% above pre-treatment values.

Further conditions of in individual capable of being treatable in accordance with the present invention are bulimia nervosa, anorexia, male erectile dysfunction, female sexual dysfunction, amelioration of ischemic nerve or muscle damage, as well as systemic lupus erythematosus.

Subcutaneous Administration

It is important to note that ghrelin receptors are found in a number of places in the organism besides on the GH producing cells and in the hypothalamic centers for appetite etc. control. In the CNS these receptors are tuned to receiving signals from local ghrelin containing neurons. Peripherally secreted or artificially administered ghrelin will not reach such sites due to the blood brain barrier, however currently available so-called GH secretagouges, which are small organic compounds such as MK-0677, will pass the blood brain barrier and also reach these sites—and consequently have the danger of causing unwanted side effects. Thus such compounds which do have the advantage of being for example orally active will not be optimal for mimicking the natural pre-meal, appetite inducing surge of ghrelin, since they will reach basically all ghrelin receptors in the body. In contrast, by using the natural peptide, ghrelin itself or homologues thereof, and administering it peripherally—as in a preferred embodiment of the present invention—it is ensured that only the relevant, appetite regulating ghrelin receptors are reached and stimulated.

Any parenteral administration form that will ensure that the ghrelin receptors which normally are the target for peripherally produced ghrelin in the premeal situation will be exposed to sufficient levels of the bioactive form of ghrelin to ensure robust and appropriate appetite stimulation, without causing desensitization of the system, may be part of the present invention. However, taken into consideration that the individuals to be treated possibly will have to receive treatment for a longer period, such as weeks or months, it is preferred that the administration form is well suited herefor.

Accordingly, it is preferred that the secretagogue, such as a ghrelin-like compound, according to the invention is administered subcutaneously in an amount sufficient to allow sufficient levels of the bioactive form of ghrelin, i.e. the acylated form, to reach the receptors in time, such as prior to the forthcoming meal.

The present invention preferably deals with methods for administering a secretagogue, such as ghrelin, in a way which mimics the physiologically pre-meal situation as closely as possible yet providing patients in need of increased food intake, for example fragile elderly, post operative patients, patients with lost appetite as part of cachexia for example precipitated by cancer, cardiac disease etc. with a sufficient extra stimulatory input to their appetite regulating ghrelin receptors, which normally are reached by ghrelin in the pre-meal situation.

Bolus Administration

Furthermore, from a molecular pharmacological point-of-view it is important to note that it has been found that the ghrelin receptor normally is exposed to short-lived surges in the concentrations of the natural agonist ligand, ghrelin. The GHS-R 1a receptor belongs to the class of receptors, so-called G protein coupled receptors or 7TM receptors, that upon continued exposure to an agonist will be desensitizised, internalized and down-regulated. These mechanisms, which are inherent to the overall signal transduction system, involve processes such as receptor phosphorylation (which in itself decreases the affinity of the receptor for the agonist) binding of inhibitory proteins such as arrestin (which sterically block the binding of signal transduciton molecules such as G proteins). Another part of the agonist mediated desensitization process is receptor internalization (i.e. physical removal of the receptor from the cell surface where it could bind the agonist) as well as receptor down regulation (i.e. decreased production/expression of the receptor). Receptor internalization could after short-lived exposure of the receptor to agonist be followed by a resensitization process, where the receptor is dephosphorylated and recycled to the cell surface to be used again. Without being bound by theory, it is believed that, upon prolonged stimulation, which would occur for example during a long-lasting continuous infusion of the agonist, the receptor down-regulation process ensures that the target cell is adjusted in its signal transduction system etc. to this situation.

The present invention provides a procedure for an optimal administration of ghrelin to patients in order to obtain a maximal response and avoid for example desensitization mechanisms.

Accordingly, the present invention relates in one aspect to administration of a secretagogue, such as a ghrelin-like compound, in boluses, preferably a bolus prior to each main meal. It has been found, in contrary to the prolonged administration processes in the prior art, that a bolus administration leads not only to stimulation of appetite, but also to stimulation of feed intake and more important to stimulation of weight gain.

Without being bound by theory, it is believed that premeal subcutaneous injection, intravenous injection or short-term infusions of appropriate doses of a secretagogue, such as ghrelin or a ghrelin-like compound, will ensure that a robust stimulation of appetite inducing ghrelin receptors will be obtained with minimal constraint to the mobility etc. of the patient. Thus for example, patients with hip fractures can in the post operative situation be treated in the premeal period and if required during the meal as such, but will be free to move around and participate in the important post operative physicotherapeutic regimens.

In one preferred embodiment of the present invention, a secretagogue such as ghrelin or a ghrelin-like compound is administered as a bolus in an amount equivelant to 10 μg per kg body weight.

Ghrelin-like Compound

Any secretagogue, such as ghrelin or a ghrelin-like compound, may be used in the present invention. One preferred type of ghrelin-like compound according to the invention described herein is a compound comprising a structure defined by formula I:
Z¹-(X¹)ₘ—(X²)—(X³)ₙ-Z², wherein Formula I:

Z¹ is an optionally present protecting group

each X¹ is independently selected from an amino acid, wherein said amino acid is selected from naturally occurring and synthetic amino acids,

X² is any amino acid selected from naturally occurring and synthetic occurring amino acids, said amino acid being modified with a bulky hydrophobic group, preferably an acyl group, or a fatty acid,

each X³ is independently selected from an amino acid, wherein said amino acid is selected from naturally occurring and synthetic amino acids,

wherein one or more of X¹ and X³ optionally may be modified by a bulky hydrophobic group, preferably an acyl group, or a fatty acid,

Z² is an optionally present protecting group,

m is an integer in the range of from 1-10

n is 0 or an integer in the range of from 1-35.

Accordingly, the term “secretagogue” includes the naturally occurring 28 aa human ghrelin, the amino acid of which is shown in SEQ ID NO: 1, as well as the naturally occurring 27 aa human ghrelin, the amino acid of which is shown in SEQ ID NO: 2. Thus, the present invention relates to the use of ghrelin or a peptide homologous thereto. Ghrelin is described by Kojima in Nature (1999), vol. 402,656-660.

The present invention includes diastereomers as well as their racemic and resolved enantiomerically pure forms. Secretagogues can contain D-amino acids, L-amino acids, alpha-amino acid, beta-amino acid, gamma-amino acid, natural amino acid and synthetic amino acid or the like or a combination thereof. Preferably, amino acids present in a ghrelin-like compound are the L-enantiomer.

The ghrelin-like compound preferably comprises an amino acid modified with a bulky hydrophobic group. The number of amino acids N-terminally to the modified amino acid is preferably within the range of from 1-9. Accordingly, m is preferably an integer in the range of from 1-9, such as of from 1-8, such as of from 1-7, such as of from 1-6, such as of from 1-5, such as of from 1-4, such as of from 1-3, such as of from,1-2, such as 2.

It is more preferred that the number of amino acids N-terminally to the modified amino acid is low, such as of from 1-3, such as of from 1-2. Most preferably 2 amino acids are positioned N-terminal to the modified amino acid.

In a preferred embodiment (X¹)m has a Gly residue in the N-terminal part of the sequence. Accordingly, in preferred embodiment (X¹)ₘ is selected from the sequences:

Gly, Gly-Ser, Gly-Cys, Gly-Lys, Gly-Asp, Gly-Glu,
Gly-Arg, Gly-His, Gly-Asn, Gly-Gln, Gly-Thr, and
Gly-Tyr.

More preferably (X¹)ₘ is selected from, Gly-Ser, and Gly-Cys, most preferably from Gly-Ser.

In other words, in a preferred embodiment the ghrelin-like compound is selected from a compound of
Z¹-Gly-(X¹)ₘ₋₁—(X²)—(X³)ₙ-Z², formula II
Z¹-Gly-Ser-(X²)—(X³)ₙ-Z², and formula III
Z¹-Gly-(X²)—(X³)ₙ-Z². formula IV

And more preferably the ghrelin-like compound has formula III.

As described above, X² may be any amino acid modified with a bulky hydrophobic group. In particular X² is selected from the group of modified Ser, Cys, Asp, Lys, Trp, Phe, Ile, and Leu. More preferably x² is selected from the group of modified Ser, modified Cys and modified Lys, and most preferably X² is modified Ser.

Furthermore, (X¹)ₘ—(X²) is preferably Gly-Xaa-Ser*, or Gly-Xaa-Cys*, wherein Xaa is any amino acid, more preferably (X¹)ₘ—(X²) is Gly-Ser-Ser*, or Gly-Ser-Cys*, wherein * indicates that the amino acid residue is modified with a bulky hydrophobic group.

(X³)n preferably comprises a sequences which is a fragment of ghrelin, such as human ghrelin. Accordingly, (X³)ₙ preferably comprises a sequence selected from one or more of the sequences shown below:

Phe Leu Ser Pro Glu His Gln
Phe Leu Ser Pro Glu His
Phe Leu Ser Pro Glu
Phe Leu Ser Pro
Phe Leu Ser
Phe Leu
Phe

In a preferred embodiment the length of the ghrelin-like compound is substantially similar to the length of human ghrelin, i.e. 27 or 28 amino acids. Accordingly, n is preferably an integer in the range of from 1-25, such as of from 1-24, such as from 1-15, such as of from 1-10, or such as of from 10-25, such as of from 10-24, such as of from 15-25, such as of from 15-24.

Most preferably a ghrelin-like compound includes the naturally occurring 28 aa human ghrelin, the amino acid of which is shown in SEQ ID NO: 1, as well as the naturally occurring 27 aa human ghrelin, the amino acid of which is shown in SEQ ID NO: 2.

(X³)ₙ may be selected from any fragment of ghrelin, such as human ghrelin, and accordingly, (X³)ₙ may be selected from one or more of the sequences shown below or a homologue thereof:

Phe Leu Ser Pro Glu His Gln Arg Val Gln Gln Arg
Lys Glu Ser Lys Lys Pro Pro
Ala Lys Leu Gln Pro Arg
Phe Leu Ser Pro Glu His Gln Arg Val Gln Gln Arg
Lys Glu Ser Lys Lys Pro Pro
Ala Lys Leu Gln Pro
Phe Leu Ser Pro Glu His Gln Arg Val Gln Gln Arg
Lys Glu Ser Lys Lys Pro Pro
Ala Lys Leu Gln
Phe Leu Ser Pro Glu His Gln Arg Val Gln Gln Arg
Lys Glu Ser Lys Lys Pro Pro
Ala Lys Leu
Phe Leu Ser Pro Glu His Gln Arg Val Gln Gln Arg
Lys Glu Ser Lys Lys Pro Pro
Ala Lys
Phe Leu Ser Pro Glu His Gln Arg Val Gln Gln Arg
Lys Glu Ser Lys Lys Pro Pro
Ala
Phe Leu Ser Pro Glu His Gln Arg Val Gln Gln Arg
Lys Glu Ser Lys Lys Pro Pro
Phe Leu Ser Pro Glu His Gln Arg Val Gln Gln Arg
Lys Glu Ser Lys Lys Pro
Phe Leu Ser Pro Glu His Gln Arg Val Gln Gln Arg
Lys Glu Ser Lys Lys
Phe Leu Ser Pro Glu His Gln Arg Val Gln Gln Arg
Lys Glu Ser Lys
Phe Leu Ser Pro Glu His Gln Arg Val Gln Gln Arg
Lys Glu Ser
Phe Leu Ser Pro Glu His Gln Arg Val Gln Gln Arg
Lys Glu
Phe Leu Ser Pro Glu His Gln Arg Val Gln Gln Arg
Lys
Phe Leu Ser Pro Glu His Gln Arg Val Gln Gln Arg
Phe Leu Ser Pro Glu His Gln Arg Val Gln Gln
Phe Leu Ser Pro Glu His Gln Arg Val Gln
Phe Leu Ser Pro Glu His Gln Arg Val
Phe Leu Ser Pro Glu His Gln Arg
Phe Leu Ser Pro Glu His Gln
Phe Leu Ser Pro Glu His
Phe Leu Ser Pro Glu
Phe Leu Ser Pro
Phe Leu Ser
Phe Leu
Phe

Or selected from

Phe Leu Ser Pro Glu His Gln Lys Val Gln Gln Arg
Lys Glu Ser Lys Lys Pro Pro
Ala Lys Leu Gln Pro Arg
Phe Leu Ser Pro Glu His Gln Lys Val Gln Gln Arg
Lys Glu Ser Lys Lys Pro Pro
Ala Lys Leu Gln Pro
Phe Leu Ser Pro Glu His Gln Lys Val Gln Gln Arg
Lys Glu Ser Lys Lys Pro Pro
Ala Lys Leu Gln
Phe Leu Ser Pro Glu His Gln Lys Val Gln Gln Arg
Lys Glu Ser Lys Lys Pro Pro
Ala Lys Leu
Phe Leu Ser Pro Glu His Gln Lys Val Gln Gln Arg
Lys Glu Ser Lys Lys Pro Pro
Ala Lys
Phe Leu Ser Pro Glu His Gln Lys Val Gln Gln Arg
Lys Glu Ser Lys Lys Pro Pro
Ala
Phe Leu Ser Pro Glu His Gln Lys Val Gln Gln Arg
Lys Glu Ser Lys Lys Pro Pro
Phe Leu Ser Pro Glu His Gln Lys Val Gln Gln Arg
Lys Glu Ser Lys Lys Pro
Phe Leu Ser Pro Glu His Gln Lys Val Gln Gln Arg
Lys Glu Ser Lys Lys
Phe Leu Ser Pro Glu His Gln Lys Val Gln Gln Arg
Lys Glu Ser Lys
Phe Leu Ser Pro Glu His Gln Lys Val Gln Gln Arg
Lys Glu Ser
Phe Leu Ser Pro Glu His Gln Lys Val Gln Gln Arg
Lys Glu
Phe Leu Ser Pro Glu His Gln Lys Val Gln Gln Arg
Lys
Phe Leu Ser Pro Glu His Gln Lys Val Gln Gln Arg
Phe Leu Ser Pro Glu His Gln Lys Val Gln Gln
Phe Leu Ser Pro Glu His Gln Lys Val Gln
Phe Leu Ser Pro Glu His Gln Lys Val
Phe Leu Ser Pro Glu His Gln Lys

Or selected from

Phe Leu Ser Pro Glu His Gln Arg Ala Gln Gln Arg
Lys Glu Ser Lys Lys Pro Pro
Ala Lys Leu Gln Pro Arg
Phe Leu Ser Pro Glu His Gln Arg Ala Gln Gln Arg
Lys Glu Ser Lys Lys Pro Pro
Ala Lys Leu Gln Pro
Phe Leu Ser Pro Glu His Gln Arg Ala Gln Gln Arg
Lys Glu Ser Lys Lys Pro Pro
Ala Lys Leu Gln
Phe Leu Ser Pro Glu His Gln Arg Ala Gln Gln Arg
Lys Glu Ser Lys Lys Pro Pro
Ala Lys Leu
Phe Leu Ser Pro Glu His Gln Arg Ala Gln Gln Arg
Lys Glu Ser Lys Lys Pro Pro
Ala Lys
Phe Leu Ser Pro Glu His Gln Arg Ala Gln Gln Arg
Lys Glu Ser Lys Lys Pro Pro
Ala
Phe Leu Ser Pro Glu His Gln Arg Ala Gln Gln Arg
Lys Glu Ser Lys Lys Pro Pro
Phe Leu Ser Pro Glu His Gln Arg Ala Gln Gln Arg
Lys Glu Ser Lys Lys Pro
Phe Leu Ser Pro Glu His Gln Arg Ala Gln Gln Arg
Lys Glu Ser Lys Lys
Phe Leu Ser Pro Glu His Gln Arg Ala Gln Gln Arg
Lys Glu Ser Lys
Phe Leu Ser Pro Glu His Gln Arg Ala Gln Gln Arg
Lys Glu Ser
Phe Leu Ser Pro Glu His Gln Arg Ala Gln Gln Arg
Lys Glu
Phe Leu Ser Pro Glu His Gln Arg Ala Gln Gln Arg
Lys
Phe Leu Ser Pro Glu His Gln Arg Ala Gln Gln Arg
Phe Leu Ser Pro Glu His Gln Arg Ala Gln Gln
Phe Leu Ser Pro Glu His Gln Arg Ala Gln
Phe Leu Ser Pro Glu His Gln Arg Ala

Or selected from

Phe Leu Ser Pro Glu His Gln Lys Ala Gln Gln Arg
Lys Glu Ser Lys Lys Pro Pro
Ala Lys Leu Gln Pro Arg
Phe Leu Ser Pro Glu His Gln Lys Ala Gln Gln Arg
Lys Glu Ser Lys Lys Pro Pro
Ala Lys Leu Gln Pro
Phe Leu Ser Pro Glu His Gln Lys Ala Gln Gln Arg
Lys Glu Ser Lys Lys Pro Pro
Ala Lys Leu Gln
Phe Leu Ser Pro Glu His Gln Lys Ala Gln Gln Arg
Lys Glu Ser Lys Lys Pro Pro
Ala Lys Leu
Phe Leu Ser Pro Glu His Gln Lys Ala Gln Gln Arg
Lys Glu Ser Lys Lys Pro Pro
Ala Lys
Phe Leu Ser Pro Glu His Gln Lys Ala Gln Gln Arg
Lys Glu Ser Lys Lys Pro Pro
Ala
Phe Leu Ser Pro Glu His Gln Lys Ala Gln Gln Arg
Lys Glu Ser Lys Lys Pro Pro
Phe Leu Ser Pro Glu His Gln Lys Ala Gln Gln Arg
Lys Glu Ser Lys Lys Pro
Phe Leu Ser Pro Glu His Gln Lys Ala Gln Gln Arg
Lys Glu Ser Lys Lys
Phe Leu Ser Pro Glu His Gln Lys Ala Gln Gln Arg
Lys Glu Ser Lys
Phe Leu Ser Pro Glu His Gln Lys Ala Gln Gln Arg
Lys Glu Ser
Phe Leu Ser Pro Glu His Gln Lys Ala Gln Gln Arg
Lys Glu
Phe Leu Ser Pro Glu His Gln Lys Ala Gln Gln Arg
Lys
Phe Leu Ser Pro Glu His Gln Lys Ala Gln Gln Arg
Phe Leu Ser Pro Glu His Gln Lys Ala Gln Gln
Phe Leu Ser Pro Glu His Gln Lys Ala Gln
Phe Leu Ser Pro Glu His Gln Lys Ala

In another embodiment (X³)ₙ comprises or consists of a sequence selected from the sequences

Phe Leu Ser Pro Glu His Gln
Phe Leu Ser Pro Glu His
Phe Leu Ser Pro Glu
Phe Leu Ser Pro
Phe Leu Ser
Phe Leu
Phe

Functionality

The secretagogues described herein are active at the receptor for GHS as described above, i.e. the receptor GHS-R 1a. The compounds can bind to the receptor, and preferably, stimulate receptor activity.

The receptor activity can be measured using different techniques such as detecting a change in the intracellular conformation of the receptor, in the G-protein coupled activities, and/or in the intracellular messengers.

One simple measure of the ability of a ghrelin like compound to activate the ghrelin receptor is to measure its EC50, i.e. the dose at which the compound is able to activates the signalling of the receptor to half of the maximal effect of the compound. The receptor can either be expressed endogenously on primary cells cultures, for example pituitary cells, or heterologously expressed on cells transfected with the ghrelin receptor. Whole cell assays or assays using membranes prepared form either of these cell types can be used depending on the type of assay.

As the receptor is generally believed to be primarily coupled to the Gq signalling pathway, any suitable assay which monitor activity in the Gq/G11 signalling pathway can be used, for example:
1) an assay measuring the activation of Gq/G11 performed for example by measurement of GTPgS binding combined with, e.g., anti-G-alpha-q or -11 antibody precipitation in order to increase the signal to noise ratio. This assay may also detect coupling to other G-proteins than Gq/11.
2) An assay which measure the activity of phopholipase C (PLC) one of the first down-stream effector molecules in the pathway, for example by measuring the accumulation of inositol phosphate which is one of the products of PLC.
3) More down stream in the signalling cascade is the mobilization of calcium from the intracellular stores
4) Further more down stream signalling molecules such as the activity of different kinds of MAP kinases (p38, jun, ect.), NF-κ-B translocation and CRE driven gene transcription may also be measured.
5) Binding of fluorescently tagged arrestin to the activated ghrelin receptor

Examples of suitable protocols for use in determining secretagogue functionality are given in Example 5.

In one embodiment the binding of a compound to the receptor GHS-R 1A can be measured by the use of the assay described herein above.

A ghrelin-like compound according to the invention preferably has at least about 50%, at least about 60%, at least about 70%, at least about 80%, or at least about 90%, functional activity relative to 28 aa human ghrelin as determined using the assay described herein above, and/or an EC50 greater than about 1,000, greater than about 100, or greater than about 50, or greater than about 10. Greater refers to potency and thus indicates a lesser amount is needed to achieve binding inhibition.

In one embodiment of the invention, the compound has a potency (EC50) on the GHS-R 1A of less than 500 nM. In another embodiment the compound has a potency (EC50) on the GHS-R 1A of less than 100 nM, such as less than 80 nM, for example less than 60 nM, such as less than 40 nM, for example less than 20 nM, such as less than 10 nM, for example less than 5 nM, such as less than 1 nM, for example less than 0.5 nM, such as less than 0.1 nM, for example less than 0.05 nM, such as less than 0.01 nM.

In a further embodiment the dissociation constant (Kd) of the compound is less than 500 nM. In a still further embodiment the dissociation constant (Kd) of the ligand is less than 100 nM, such as less than 80 nM, for-example less than 60 nM, such as less than 40 nM, for example less than 20 nM, such as less than 10 nM, for example less than 5 nM, such as less than 1 nM, for example less than 0.5 nM, such as less than 0.1 nM, for example less than 0.05 nM, such as less than 0.01 nM.

Binding assays can be performed using recombinantly-produced receptor polypeptides present in different environments. Such environments include, for example, cell extracts and purified cell extracts containing the receptor polypeptide expressed from recombinant nucleic acid or naturally occurring nucleic acid; and also include, for example, the use of a purified GHS receptor polypeptide produced by recombinant means or from naturally occurring nucleic acid which is introduced into a different environment.

Using a recombinantly expressed GHS receptor offers several advantages such as the ability to express the receptor in a defined cell system, so that a response to a compound at the receptor can more readily be differentiated from responses at other receptors. For example, the receptor can be expressed in a cell line such as HEK 293, COS 7, and CHO not normally expressing the receptor by an expression vector, wherein the same cell line without the expression vector can act as a control.

Identity and Homology

The term “identity” or “homology” shall be construed to mean the percentage of amino acid residues in the candidate sequence that are identical with the residue of a corresponding sequence to which it is compared, after aligning the sequences and introducing gaps, if necessary to achieve the maximum percent identity for the entire sequence, and not considering any conservative substitutions as part of the sequence identity. Neither N— or C-terminal extensions nor insertions shall be construed as reducing identity or homology. Methods and computer programs for the alignment are well known in the art. Sequence identity may be measured using sequence analysis software (e.g., Sequence Analysis Software Package, Genetics Computer Group, University of Wisconsin Biotechnology Center, 1710 University Ave., Madison, Wis. 53705). This software matches similar sequences by assigning degrees of homology to various substitutions, deletions, and other modifications.

A ghrelin homologue of one or more of the sequences specified herein may vary in one or more amino acids as compared to the sequences defined, but is capable of performing the same function, i.e. a homologue may be envisaged as a functional equivalent of a predetermined sequence.

A ghrelin homologue is preferably a ghrelin-like compound as defined above.

As described above a homologue of any of the predetermined sequences herein may be defined as:
i) homologues comprising an amino acid sequence capable of being recognised by an antibody, said antibody also recognising the 28 aa human ghrelin, preferably the acylated 28 aa human ghrelin, and/or
ii) homologues comprising an amino acid sequence capable of binding selectively to GHS-R la, and/or
iii) homologues having a substantially similar or higher binding affinity to GHS-R 1a than the 28 aa human ghrelin, preferably the acylated 28 aa human ghrelin.

In the above examples, the 28 aa human ghrelin has the sequence shown in SEQ ID NO: 1, and when acylated is acylated in position 3.

The antibodies used herein may be antibodies binding the N-terminal part of ghrelin or the C-terminal part of ghrelin, preferably the N-terminal part of ghrelin. The anti-bodies may be antibodies as described in Ariyasu et al. “Delayed short-term secretory regulation of ghrelin in obese animals: Evidensed by a specific RIA for the active form of ghrelin, Endocrinology 143(9):3341-3350, 2002.

Examples of homologues comprises one or more conservative amino acid substitutions including one or more conservative amino acid substitutions within the same group of predetermined amino acids, or a plurality of conservative amino acid substitutions, wherein each conservative substitution is generated by substitution within a different group of predetermined amino acids.

Homologues may thus comprise conservative substitutions independently of one another, wherein at least one glycine (Gly) of said homologue is substituted with an amino acid selected from the group of amino acids consisting of Ala, Val, Leu, and lie, and independently thereof, homologues, wherein at least one of said alanines (Ala) of said homologue thereof is substituted with an amino acid selected from the group of amino acids consisting of Gly, Val, Leu, and lie, and independently thereof, homologues, wherein at least one valine (Val) of said homologue thereof is substituted with an amino acid selected from the group of amino acids consisting of Gly, Ala, Leu, and lie, and independently thereof, homologues thereof, wherein at least one of said leucines (Leu) of said homologue thereof is substituted with an amino acid selected from the group of amino acids consisting of Gly, Ala, Val, and lie, and independently thereof, homologues thereof, wherein at least one isoleucine (Ile) of said homologues thereof is substituted with an amino acid selected from the group of amino acids consisting of Gly, Ala, Val and Leu, and independently thereof, homologues thereof wherein at least one of said aspartic acids (Asp) of said homologue thereof is substituted with an amino acid selected from the group of amino acids consisting of Glu, Asn, and Gln, and independently thereof, homologues thereof, wherein at least one of said phenylalanines (Phe) of said homologues thereof is substituted with an amino acid selected from the group of amino acids consisting of Tyr, Trp, His, Pro, and preferably selected from the group of amino acids consisting of Tyr and Trp, and independently thereof, homologues thereof, wherein at least one of said tyrosines (Tyr) of said homologues thereof is substituted with an amino acid selected from the group of amino acids consisting of Phe, Trp, His, Pro, preferably an amino acid selected from the group of amino acids consisting of Phe and Trp, and independently thereof, homologues thereof, wherein at least one of said arginines (Arg) of said fragment is substituted with an amino acid selected from the group of amino acids consisting of Lys and His, and independently thereof, homologues thereof, wherein at least one lysine (Lys) of said homologues thereof is substituted with an amino acid selected from the group of amino acids consisting of Arg and His, and independently thereof, homologues thereof, wherein at least one of said aspargines (Asn) of said homologues thereof is substituted with an amino acid selected from the group of amino acids consisting of Asp, Glu, and Gln, and independently thereof, homologues thereof, wherein at least one glutamine (Gln) of said homologues thereof is substituted with an amino acid selected from the group of amino acids consisting of Asp, Glu, and Asn, and independently thereof, homologues thereof, wherein at least one proline (Pro) of said homologues thereof is substituted with an amino acid selected from the group of amino acids consisting of Phe, Tyr, Trp, and His, and independently thereof, homologues thereof, wherein at least one of said cysteines (Cys) of said homologues thereof is substituted with an amino acid selected from the group of amino acids consisting of Asp, Glu, Lys, Arg, His, Asn, Gln, Ser, Thr, and Tyr.

Conservative substitutions may be introduced in any position of a preferred predetermined sequence. It may however also be desirable to introduce non-conservative substitutions, particularly, but not limited to, a non-conservative substitution in any one or more positions.

A non-conservative substitution leading to the formation of a functionally equivalent homologue of the sequences herein would for example i) differ substantially in polarity, for example a residue with a non-polar side chain (Ala, Leu, Pro, Trp, Val, Ile, Leu, Phe or Met) substituted for a residue with a polar side chain such as Gly, Ser, Thr, Cys, Tyr, Asn, or Gln or a charged amino acid such as Asp, Glu, Arg, or Lys, or substituting a charged or a polar residue for a non-polar one; and/or ii) differ substantially in its effect on polypeptide backbone orientation such as substitution of or for Pro or Gly by another residue; and/or iii) differ substantially in electric charge, for example substitution of a negatively charged residue such as Glu or Asp for a positively charged residue such as Lys, His or Arg (and vice versa); and/or iv) differ substantially in steric bulk, for example substitution of a bulky residue such as His, Trp, Phe or Tyr for one having a minor side chain, e.g. Ala, Gly or Ser (and vice versa).

Substitution of amino acids may in one embodiment be made based upon their hydrophobicity and hydrophilicity values and the relative similarity of the amino acid side-chain substituents, including charge, size, and the like. Exemplary amino acid substitutions which take various of the foregoing characteristics into consideration are well known to those of skill in the art and include: arginine and lysine; glutamate and aspartate; serine and threonine; glutamine and asparagine; and valine, leucine and isoleucine.

In a preferred embodiment the binding domain comprises a homologue having an amino acid sequence at least 60% homologous to SEQ ID NO 1.

More preferably the homology is at least 65%, such as at least 70% homologous, such as at least 75% homologous, such as at least 80% homologous, such as at least 85% homologous, such as at least 90% homologous, such as at least 95% homologous, such as at least 98% homologous to SEQ ID NO 1.

In a more preferred embodiment the percentages mentioned above relates to the identity of the sequence of a homologue as compared to SEQ ID NO 1.

Homologues to SEQ ID NO: 1 may be 27 aa human ghrelin SEQ ID NO: 2, rat ghrelin SEQ ID NO: 3. Additional preferred sequences are listed in EP 1 197 496 (Kangawa).

Other homologues are the variants described in EP 1197496 (Kangawa) incorporated herein by reference.

Bulky Hydrophobic Group

The bulky hydrophobic group of the secretagogue according to the invention is any bulky hydrophobic group capable of providing the des-acylated 28 aa human ghrelin, or an analogue thereof, with binding affinity to GHS-R 1a. Any suitable amino acid may be modified with any suitable bulky hydrophobic group; in a preferred embodiment, a Ser residue (preferably amino acid number 3 in the amino acid chain) is modified with the bulky hydrophobic group.

When the amino acid being modified contains e.g. —OH, —SH, —NH or —NH₂ as a substituent group in a side chain thereof, a group formed by acylating such a substituent group is preferred. The mode of linkage may thus be selected from the group consisting of ester, ether, thioester, thioether, amide and carbamide.

For example, if the modified amino acid is serine, threonine, tyrosine or oxyproline, the amino acid has a hydroxyl group in the side chain. If the modified amino acid is cysteine, the amino acid has a mercapto group in the side chain. If the modified amino acid is lysine, arginine, histidine, tryptophan, proline oroxyproline, it has an amino group or imino group in the side chain.

The hydroxyl group, mercapto group, amino group and imino group described above may thus have been chemically modified. That is, the hydroxyl group or mercapto group may be etherized, esterified, thioetherified or thioesterified. The imino group may have been iminoetherified, iminothioetherified or alkylated. The amino group may have been amidated, thioamidated or carbamidated.

Further, the mercapto group may have been disulfidated, the imino group may have been amidated or thioamidated, and the amino group may have been alkylated or thiocarbamidated.

In a preferred embodiment the modified amino acid is Ser coupled through an ester linkage to the hydrophobic group.

The hydrophobic group may be any group with a saturated or unsaturated alkyl or acyl group containing one or more carbon atoms. In one embodiment the bulky hydrophobic group is an acyl group, including groups formed by removing a hydroxyl group from an organic carboxylic acid, organic sulfonic acid or organic phosphoric acid. The organic carboxylic acid includes e.g. fatty acids, and the number of carbon atoms thereof is preferably 1 to 35. In the organic sulfonic acid or organic phosphoric acid, the number of carbon atoms thereof is preferably 1 to 35.

Accordingly, the acyl group is preferably selected from a C1-C35 acyl group, such as a C1-C20 acyl group, such as a C1-C15 acyl group, such as a C6-C15 acyl group, such as a C6-C12 acyl group, such as a C8-C12 acyl group.

More preferably the acyl group is selected from the group of C7 acyl group, C8 acyl group, C9 acyl group, C10 acyl group, C11 acyl group, and C12 acyl group. Such acyl group may be formed from octanoic acid (preferably caprylic acid), decanoic acid (preferably capric acid), or dodecanoic acid (preferably lauric acid), as well as monoene or polyene fatty acids thereof.

In one embodiment the acyl group is selected from the group of C8 acyl group, and C10 acyl group. Such acyl groups may be formed from octanoic acid (preferably caprylic acid), or decanoic acid (preferably capric acid).

In another embodiment the acyl group is selected from the group of C7 acyl group, C9 acyl group, and Cl1 acyl group, such as from the group of C9 acyl group and C11 acyl group.

Furthermore, the modified amino acid may be any amino acid wherein a group is modified as described in EP 1 197 496 (Kangawa), which is hereby incorporated by reference.

Protecting Group

The ghrelin-like compound according to the invention may comprise a protecting group at the N-terminus or the C-terminus or at both.

A protecting group covalently joined to the N-terminal amino group reduces the reactivity of the amino terminus under in vivo conditions. Amino protecting groups include —C1-10 alkyl, —C1-10 substituted alkyl, —C2-10 alkenyl, -C2-10 substituted alkenyl, aryl, —C1-6 alkyl aryl, —C(O)—(CH2)1-6—COOH, —C(O)—C1-6 alkyl, —C(O)-aryl; —C(O)—O—C1-6 alkyl, or —C(O)—O-aryl. Preferably, the amino terminus protecting group is acetyl, propyl, succinyl, benzyl, benzyloxycarbonyl or tbutyloxycarbonyl.

A protecting group covalently joined to the C-terminal carboxy group reduces the reactivity of the carboxy terminus under in vivo conditions. The carboxy terminus protecting group is preferably attached to the a-carbonyl group of the last amino acid. Carboxy terminus protecting groups include amide, methylamide, and ethylamide.

Conjugates

The secretagogue, such as a ghrelin-like compound, to be used in the present invention may be provided in the form of a secretagogue conjugate, i.e. a molecule comprising the secretagogue conjugated to another entity.

The other entity may be any substance that is capable of conferring improved properites to the secretagogue, e.g. in terms of improved stability, half-life, etc. Examples of suitable entities are described in the following.

For example the secretagogue may be conjugated to a peptide, such as a peptide having effect on nociceptin receptor ORL1. In one embodiment the conjugate is a conjugate of ghrelin or a derivative or homologue thereof and a peptide having effect on ORL1, e.g. the peptide Ac—RYY(RK)(WI)RK)—NH₂, where the brackets show allowable variation of amino acid residues. Examples of other suitable peptides are found in US patent applications 2003040472 and US2002004483, and U.S. Pat. No. 5,869,046.

In another embodiment of the present invention, a secretagogue, such as ghrelin or a ghrelin-like compound, is conjugated to a polymer molecule. The polymer molecule may be any suitable polymer molecule, such as a natural or synthetic polymer, typically with a molecular weight in the range of about 1-100 kDa, such as about 3-20, kDa, e.g. 5-10 kDa. The polymer is attached to a reactive group present on the secretagogue, e.g. an amine group or a thiol group.

Examples of suitable polymer molecules include polymer molecules selected from the group consisting of polyalkylene oxide (PAO), including polyalkylene glycol (PAG), such as linear or branched polyethylene glycol (PEG) and polypropylene glycol (PPG), poly-vinyl alcohol (PVA), poly-carboxylate, poly-(vinylpyrolidone), polyethylene-co-maleic acid anhydride, polystyrene-co-maleic acid anhydride, dextran, including carboxymethyl-dextran. Preferably, the polymer molecule is a PEG molecule, in particular a monofunctional PEG, such as methoxypolyethylene glycol (mPEG). Suitable activated PEG molecules are available from Nektar Therapeutics Inc. (Huntsville Ala., US) or from Valentis, Inc., Burlingame, Calif. U.S.A. Alternatively, the polymer molecules can be activated by conventional methods known in the art, e.g., as disclosed in WO 90/13540. Specific examples of activated PEG polymers include the following linear PEGs: NHS-PEG (e.g., SPA-PEG, SSPA-PEG, SBA-PEG, SS-PEG, SSA-PEG, SC-PEG, SG-PEG, and SCM-PEG), and NOR-PEG), BTC-PEG, EPOX-PEG, NCO-PEG, NPC-PEG, CDI-PEG, ALD-PEG, TRES-PEG, VS-PEG, IODO-PEG, and MAL-PEG, and branched PEGs such as PEG2-NHS and those disclosed in U.S. Pat. No. 5,932,462 and U.S. 5,643,575, both of which are incorporated herein by reference.

The PEGylation (i.e. conjugation of the secretagogue polypeptide and the activated polymer molecule) is conducted in accordance with established procedures, e.g., as described in the following references (which also describe suitable methods for activation of polymer molecules): R. F. Taylor, (1991), “Protein immobilisation. Fundamental and applications”, Marcel Dekker, N.Y.; S. S. Wong, (1992), “Chemistry of Protein Conjugation and Crosslinking”, CRC Press, Boca Raton; G. T. Hermanson et al., (1993), “Immobilized Affinity Ligand Techniques”, Academic Press, N.Y.).

It is also contemplated according to the invention to couple the polymer molecules to the secretagogue through a linker. Suitable linkers are well known to the skilled person. A preferred example is cyanuric chloride (Abuchowski et al., (1977), J. Biol. Chem., 252, 3578-3581; U.S. Pat. No. 4,179,337; Shafer et al., (1986), J. Polym. Sci. Polym. Chem. Ed., 24, 375-378.

In yet another embodiment the secretagogue is conjugated to an oligosaccharide molecule, such as dextran, glycan, transferrin, etc. Such conjugation may be achieved in accordance with established technologies, e.g. those available from Neose Technologies, Inc. Horsham, Pa.

In yet another embodiment, the secretagogue is conjugated to an Fc region of an IgG molecule, typically in the form of a fusion protein. For instance, a salvage receptor binding epitope of the Fc region of an IgG (i.e. the Fc portion of an immunoglobulin of the isotype IgG) is incorporated into the secretagogue so as to increase its circulatory half-life, but so as not to lose its biological activity. This can take place by any means, such as by mutation of the appropriate region in the secretagogue to mimic the Fc region or by incorporating the epitope into a peptide tag that is then fused to the secretagogue at either end or in the middle or by DNA or peptide synthesis.

The salvage receptor binding epitope is any suitable such epitope as known to the person skilled in the art, and its nature will depend, e.g., on the type of secretagogue being modified. The epitope is introduced into the secretagogue such that the biological activity of the secretagogue is maintained, i.e., the epitope does not adversely affect the conformation of the secretagogue or affect its binding to ligands that confers its biological activity.

Alternatively to providing the secretagogue in the form of a conjugate, the secretagogue may be modified to include suitable reactive groups, whereby the thus modified secretagogue is capable of forming a conjugate in vivo (after having been administered to an individual) through covalent bonding with available reactive functionalities on blood components. The invention also relates to such modified secretogogues, and methods for their use. Also, the invention relates to conjugates formed in vitro between a modified secretagogue as described above and a blood component. The conjugates formed in accordance with this embodiment are contemplated to have an increased in vivo half life as compared to the corresponding non-modified secretagogue.

In accordance with this embodiment, the secretagogue is modified with a chemically reactive group (reactive entity). The reactive entity may, e.g., be selected from the wide variety of active carboxyl groups, particularly esters, where the hydroxyl moiety is physiologically acceptable. Such groups may be selected from the group consisting of N-hydroxysuccinimide (NHS), N-hydroxy-sulfosuccinimide (sulfo-NHS), maleimide-benzoyl-succinimide (MBS), gamma-maleimido-butyryloxy succinimide ester (GMBS) and maleimidopropionic acid (MPA). The principal targets for this group of entities are primary amines on the blood component. Another group of active entities is constituted by a maleimido-containing group such as MPA and gamma-maleimide-butrylamide (GMBA) Such groups react with thiol groups present on the blood component.

The blood component with which the modified secretagogue is designed to react may be any blood component having an available target group, e.g. an amine or a thiol group, and which is suitable as a carrier for binding the modified secretagogue in vivo and thereby extend the circulating half-life thereof. Examples of such blood components are serum albumin and IgG.

As mentioned above the covalent bonding of a modified secretagogue to a blood component may be achieved in vivo by administration of the modified secretagogue directly to the patient. The administration may be done in any suitable form, such as in the form of a bolus or introduced slowly over time by infusion using metered flow or the like. Alternatively, the secretagogue/blood component conjugate may also be prepared ex vivo by combining blood with the modified secretagogue, allowing covalent bonding of the modified secretagogueto reactive functionalities on blood components and then returning or administering the conjugated blood to the individual. Moreover, the above may also be accomplished by first purifying an individual blood component or limited number of components, such as red blood cells, immunoglobulins, serum albumin, or the like, and combining the component or components ex vivo with the chemically reactive secretagogues.

Method for Production

Ghrelin-like compounds can be produced using techniques well known in the art. For example, a polypeptide region of a ghrelin-like compound can be chemically or biochemically synthesized and modified. Techniques for chemical synthesis of polypeptides are well known in the art. (See e. g., Vincent in Peptide and Protein Drug Delivery, New York, N.Y., Dekker, 1990.) Examples of techniques for biochemical synthesis involving the introduction of a nucleic acid into a cell and expression of nucleic acids are provided in Ausubel, Current Protocols in Molecular Biology, John Wiley, 1987-1998, and Sambrook et al., in Molecular Cloning, A Laboratory Manual, 2 d Edition, Cold Spring Harbor Laboratory Press, 1989.

Pharmaceutical Composition

While it is possible for the compounds or salts of the present invention to be administered as the raw chemical, it is preferred to present them in the form of a pharmaceutical composition. According, in one aspect the invention relates to a pharmaceutical composition comprising a ghrelin-like compound as defined in formula I.

In one preferred embodiment, the bulky hydrophobic group is an acyl group selected from the group of C7 acyl group, C9 acyl group, and C11 acyl group, such as from the group of C9 acyl group and C11 acyl group.

In another embodiment the invention relates to a pharmaceutical composition comprising a mixture of at least two different ghrelin-like compounds, such as a mixture of a ghrelin-like compound being acylated with a C8 acyl and a ghrelin-like compound being acylated with a C10 acyl. Without being bound by theory it is believed that such a mixture will have a longer half-life in plasma.

In yet another embodiment, the pharmaceutical composition comprises acylated ghrelin-like compounds, optionally compounds having different acyl chain lengths preferably selected from the group of C7 acyl group, C9 acyl group, and C11 acyl group, such as from the group of C9 acyl group and C11 acyl group, further optionally in combination with a desacylated Ghrelin-like compound.

Another aspect the invention relates to a pharmaceutical composition comprising any secretagogue, such as any ghrelin-like compound as defined above or a pharmaceutically acceptable salt thereof and pharmaceutically acceptable carriers, vehicles and/or excipients said composition further comprising transport molecules. The transport molecules are primarily added in order to increase the half-life of the acylated compound, preventing premature des-acylation, since the des-acylated ghrelin is not active at the GHS-R la.

Transport molecules act by having incorporated into or anchored to it the compound according to the invention.

Any suitable transport molecules known to the skilled person may be used. Examples of transport molecules are those described in the conjugate section. Other preferred examples are liposomes, micelles, and/or microspheres.

Conventional liposomes are typically composed of phospholipids (neutral or negatively charged) and/or cholesterol. The liposomes are vesicular structures based on lipid bilayers surrounding aqueous compartments. They can vary in their physio-chemical properties such as size, lipid composition, surface charge and number and fluidity of the phospholipids bilayers. The most frequently used lipid for liposome formation are: 1,2-Dilauroyl-sn-Glycero-3-Phosphocholine (DLPC), 1,2-Dimyristoylsn-Glycero-3-Phosphocholine (DMPC), 1,2-Dipalmitoyl-sn-Glycero-3-Phosphocholine (DPPC), 1,2-Distearoyl-sn-Glycero-3-Phosphocholine (DSPC), 1,2-Dioleoyl-sn-Glycero-3-Phosphocholine (DOPC), 1,2-Dimyristoyl-sn-Glycero-3-Phosphoethanolamine (DMPE), 1,2-Dipalmitoyl-sn-Glycero-3-Phosphoethanolamine (DPPE), 1,2-Dioleoyl-sn-Glycero-3-Phosphoethanolamine (DOPE), 1,2-Dimyristoyl-sn-Glycero-3-Phosphate (Monosodium Salt) (DMPA), 1,2-Dipalmitoyl-sn-Glycero-3-Phosphate (Monosodium Salt) (DPPA), 1,2-Dioleoyl-sn-Glycero-3-Phosphate (Monosodium Salt) (DOPA), 1,2-Dimyristoyl-sn-Glycero-3-[Phospho-rac-(1-glycerol)] (Sodium Salt) (DMPG), 1,2-Dipalmitoyl-sn-Glycero-3-[Phospho-rac-(1-glycerol)] (Sodium Salt) (DPPG), 1,2-Dioleoyl-sn-Glycero-3-[Phospho-rac-(1-glycerol)] (Sodium Salt) (DOPG), 1,2-Dimyristoyl-sn-Glycero-3-[Phospho-L-Serine] (Sodium Salt) (DMPS), 1,2-Dipalmitoyl-sn-Glycero-3-[Phospho-L-Serine) (Sodium Salt) (DPPS), 1,2-Dioleoyl-sn-Glycero-3-[Phospho-L-Serine] (Sodium Salt) (DOPS), 1,2-Dioleoyl-sn-Glycero-3-Phosphoethanolamine-N-(glutaryl) (Sodium Salt) and 1,1′,2,2′-Tetramyristoyl Cardiolipin (Ammonium Salt). Formulations composed of DPPC in combination with other lipid or modifiers of liposomes are preferred e.g. in combination with cholesterol and/or phosphatidylcholine.

Long-circulating liposomes are characterized by their ability to extravasate at body sites where the permeability of the vascular wall is increased. The most popular way to produce long circulating liposomes is to attach hydrophilic polymer polyethylene glycol (PEG) covalently to the outer surface of the liposome. Some of the preferred lipids are: 1,2-Dipalmitoyl-sn-Glycero-3-Phosphoethanolamine-N-[Methoxy(Polyethylene glycol)-2000] (Ammonium Salt), 1,2-Dipalmitoyl-sn-Glycero-3-Phosphoethanolamine-N-[Methoxy(Polyethylene glycol)-5000] (Ammonium Salt), 1,2-Dioleoyl-3-Trimethylammonium-Propane (Chloride Salt) (DOTAP). Possible lipid applicable for liposomes are supplied by Avanti, Polar lipids, Inc, Alabaster, Ala. Additionally, the liposome suspension may include lipid-protective agents which protect lipids against free-radical and lipid-peroxidative damages on storage. Lipophilic free-radical quenchers, such as alpha-tocopherol and water-soluble iron-specific chelators, such as ferrioxianine, are preferred.

A variety of methods are available for preparing liposomes, as described in, e.g., Szoka et al., Ann. Rev. Biophys. Bioeng. 9:467 (1980), U.S. Pat. Nos. 4,235,871, 4,501,728 and 4,837,028, all of which are incorporated herein by reference. One methode is described in example 9. Another method produces multilamellar vesicles of heterogeneous sizes. In this method, the vesicle-forming lipids are dissolved in a suitable organic solvent or solvent system and dried under vacuum or an inert gas to form a thin lipid film. If desired, the film may be redissolved in a suitable solvent, such as tertiary butanol, and then lyophilized to form a more homogeneous lipid mixture which is in a more easily hydrated powderlike form. This film is covered with an aqueous solution of the targeted drug and the targeting component and allowed to hydrate, typically over a 15-60 minute period with agitation. The size distribution of the resulting multilamellar vesicles can be shifted toward smaller sizes by hydrating the lipids under more vigorous agitation conditions or by adding solubilizing detergents such as deoxycholate. Additionally, the liposome suspension may include lipid-protective agents which protect lipids against free-radical and lipid-peroxidative damages on storage. Lipophilic free-radical quenchers, such as alpha-tocopherol and water-soluble iron-specific chelators, such as ferrioxianine, are preferred.

Micelles are formed by surfactants (molecules that contain a hydrophobic portion and one or more ionic or otherwise strongly hydrophilic groups) in aqueous solution. As the concentration of a solid surfactant increases, its monolayers adsorbed at the air/water or glass/water interfaces become so tightly packed that further occupancy requires excessive compression of the surfactant molecules already in the two monolayers. Further increments in the amount of dissolved surfactant beyond that concentration cause amounts equivalent to the new molecules to aggregate into micelles. This process begins at a characteristic concentration called “critical micelle concentration”.

The shape of micelles formed in dilute surfactant solutions is approximately spherical. The polar head groups of the surfactant molecules are arranged in an outer spherical shell whereas their hydrocarbon chains are oriented toward the center, forming a spherical core for the micelle. The hydrocarbon chains are randomly coiled and entangled and the micellar interior has a nonpolar, liquid-like character. In the micelles of polyoxyethylated nonionic detergents, the polyoxyethlene moieties are oriented outward and permeated by water. This arrangement is energetically favorable since the hydrophilic head groups are in contact with water and the hydrocarbon moieties are removed from the aqueous medium and partly shielded from contact with water by the polar head groups. The hydrocarbon tails of the surfactant molecules, located in the interior of the micelle, interact with one another by weak van der Waals forces.

The size of a micelle or its aggregation number is governed largely by geometric factors. The radius of the hydrocarbon core cannot exceed the length of the extended hydrocarbon chain of the surfactant molecule. Therefore, increasing the chain length or ascending homologous series increases the aggregation number of spherical micelles. If the surfactant concentration is increased beyond a few percent and if electrolytes are added (in the case of ionic surfactants) or the temperature is raised (in the case of nonionic surfactants), the micelles increase in size. Under these conditions, the micelles are too large to remain spherical and become ellipsoidal, cylindrical or finally lamellar in shape.

Common surfactants well known to one of skill in the art can be used in the micelles of the present invention. Suitable surfactants include sodium laureate, sodium oleate, sodium lauryl sulfate, octaoxyethylene glycol monododecyl ether, octoxynol 9 and PLURONIC F-127 (Wyandotte Chemicals Corp.). Preferred surfactants are nonionic polyoxyethylene and polyoxypropylene detergents compatible with IV injection such as, TWEEN-80., PLURONIC F-68., n-octyl-.beta.-D-glucopyranoside, and the like. In addition, phospholipids, such as those described for use in the production of liposomes, may also be used for micelle formation.

In another preferred embodiment, the compounds of the present invention are formulated as described in the literature for an administration route selected from: buccal delivery, sublingual delivery, transdermal delivery, inhalation and needle-free injection, such as using the methods developed by Powderjet.

For inhalation, the compounds of the present invention can be formulated as using methods known to those skilled in the art, for example an aerosol, dry powder or solubolized such as in microdroblets, preferably in a device intended for such delivery (such as commercially available from Aradigm, Alkerme or Nektar).

Administration

Suitable dosing regimens for the various compounds and methods of the present invention are preferably determined taking into account factors well known in the art including type of subject being dosed; age, weight, sex and medical condition of the subject; the route of administration; the renal and hepatic function of the subject; the desired effect; and the particular compound employed.

Optimal precision in achieving concentrations of drug within the range that yields efficacy without toxicity requires a regimen based on the kinetics of the drug's availability to target sites. This involves a consideration of the distribution, equilibrium, and elimination of a drug.

As described above, in one aspect of the invention, the secretagogue, such as ghrelin or a ghrelin-like compound, is administered subcutaneously.

In another aspect the secretagogue, such as ghrelin or a ghrelin-like compound, is administered as a premeal bolus, wherein the administration form may be any suitable parenteral form.

In a preferred embodiment the secretagogue, such as ghrelin or a ghrelin-like compound, is administered subcutaneously in a premeal bolus.

The secretagogue, such as ghrelin or a ghrelin-like compound, can also be administered during a meal as a bolus. The mode of administration during a meal includes subcutaneous administration, such as a subcutaneously administered bolus.

Pharmaceutical compositions for parenteral administration include sterile aqueous and non-aqueous injectable solutions, dispersions, suspensions or emulsions, as well as sterile powders to be reconstituted in sterile injectable solutions or dispersions prior to use.

Other suitable administration forms include suppositories, sprays, ointments, cremes, gels, inhalants, dermal patches, implants, pills, tablets, lozenges and capsuls.

A typical dosage of a compound employed according to the invention is in a concentration equivalent to from 10 ng to 10 mg ghrelin per kg bodyweight. The concentrations and amounts herein are given in equivalents of amount ghrelin, wherein the ghrelin is the 28 aa human ghrelin. Equivalents may be tested as described in the section entitled “Functionality”, above.

In a preferred embodiment the medicament is administered in a concentration equivalent to from 0.1 μg to 1 mg ghrelin per kg bodyweight, such as from 0.5 μg to 0.5 mg ghrelin per kg bodyweight, such as from 1.0 μg to 0.1 mg ghrelin per kg bodyweight, such as from 1.0 μg to 50 μg ghrelin per kg bodyweight, such as from 1.0 μg to 10 μg ghrelin per kg bodyweight.

As described above, the secretagogue, such as ghrelin or a ghrelin-like compound, is preferably administered as a bolus. Accordingly, in one embodiment the medicament is administered as a bolus prior to a meal, said bolus comprising an amount of the secretagogue or a salt thereof equivalent to from 0.3 μg to 600 mg ghrelin. More preferably, the medicament is administered as a bolus prior to a meal, said bolus comprising an amount of the secretagogue or a salt thereof equivalent to from 2.0 μg to 200 mg ghrelin, such as from 5.0 μg to 100 mg ghrelin, such as from 10 μg to 50 mg ghrelin, such as from 10 μg to 5 mg ghrelin, such as from 10 μg to 1.0 mg ghrelin.

It should be noted that the normal ghrelin response which occurs before a meal is a short-lived surge in plasma concentrations of ghrelin and that due to the relative short half life of the peptide an i.v. injection of ghrelin will ensure that a similar short-lived peak on ghrelin concentrations can be obtained. The administration route must ensure that the non-degraded, bioactive form of the peptide will be the dominating form in the circulation, which will reach the ghrelin receptors and stimulate these. Thus, in order to obtain the maximum effect of the medicament it is preferably administered from one to three times daily, each administration being within 45 minutes of a meal, such as within 30 minutes of a meal, such as within 25 minutes of a meal, such as within 20 minutes of a meal, such as within 15 minutes of a meal, such as within 10 minutes of a meal, such as within 5 minutes of a meal. More preferred the medicament is administered prior to each main meal, such as administered three times daily.

The compounds of the present invention may be formulated for nasal administration. The solutions or suspensions are applied directly to the nasal cavity by conventional means, for example with a dropper, pipette or spray. The compositions may be provided in a single or multidose form. In the latter case of a dropper or pipette this may be achieved by the patient administering an appropriate, predetermined volume of the solution or suspension. In the case of a spray this may be achieved for example by means of a metering atomizing spray pump.

The compounds of the present invention may be formulated for aerosol administration, particularly to the respiratory tract and including intranasal administration. The compound will generally have a small particle size for example of the order of 5 microns or less. Such a particle size may be obtained by means known in the art, for example by micronization. The active ingredient is provided in a pressurized pack with a suitable propellant such as a chlorofluorocarbon (CFC) for example dichlorodifluoromethane, trichlorofluoromethane, or dichlorotetrafluoroethane, carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

Compositions administered by aerosols may be prepared, for example, as solutions in saline, employing benzyl alcohol or other suitable preservatives, absorption promoters to enhance bioavailability, employing fluorocarbons, and/or employing other solubilizing or dispersing agents.

Compositions for Oral Administration

Those secretagogue types capable of remaining biologically active in an individual after oral administration (such as e.g. small molecules and short peptides) can be formulated in a wide range of oral administration dosage forms. The pharmaceutical compositions and dosage forms may comprise the compounds of the invention or its pharmaceutically acceptable salt or a crystal form thereof as the active component. The pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, wetting agents, tablet disintegrating agents, or an encapsulating material.

Preferably, the composition will be about 0.5% to 75% by weight of a compound or compounds of the invention, with the remainder consisting of suitable pharmaceutical excipients. For oral administration, such excipients include pharmaceutical grades of mannitol, lactose, starch, magnesium stearate, sodium saccharine, talcum, cellulose, glucose, gelatin, sucrose, magnesium carbonate, and the like.

In powders, the carrier is a finely divided solid which is a mixture with the finely divided active component. In tablets, the active component is mixed with the carrier having the necessary binding capacity in suitable proportions and compacted in the shape and size desired. The powders and tablets preferably containing from one to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the composition of the active compound with encapsulating material as carrier providing a capsule in which the active component, with or without carriers, is surrounded by a carrier, which is in association with it. Similarly, cachets and lozenges are included. Tablets, powders, capsules, pills, cachets, and lozenges can be as solid forms suitable for oral administration.

Drops according to the present invention may comprise sterile or non-sterile aqueous or oil solutions or suspensions, and may be prepared by dissolving the active ingredient in a suitable aqueous solution, optionally including a bactericidal and/or fungicidal agent and/or any other suitable preservative, and optionally including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container aseptically. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Other forms suitable for oral administration include liquid form preparations including emulsions, syrups, elixirs, aqueous solutions, aqueous suspensions, toothpaste, gel dentrifrice, chewing gum, or solid form preparations which are intended to be converted shortly before use to liquid form preparations. Emulsions may be prepared in solutions in aqueous propylene glycol solutions or may contain emulsifying agents such as lecithin, sorbitan monooleate, or acacia. Aqueous solutions can be prepared by dissolving the active component in water and adding suitable colorants, flavors, stabilizing and thickening agents. Aqueous suspensions can be prepared by dispersing the finely divided active component in water with viscous material, such as natural or synthetic gums, resins, methylcellulose, sodium carboxymethylcellulose, and other well known suspending agents. Solid form preparations include solutions, suspensions, and emulsions, and may contain, in addition to the active component, colorants, flavors, stabilizers, buffers, artificial and natural sweeteners, dispersants, thickeners, solubilizing agents, and the like.

Compositions for Parenteral Administration

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water. Aqueous solutions should be suitably buffered if necessary, and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Solutions of ghrelin or a ghrelin-like compound or pharmaceutically acceptable salt thereof, (and for example antigenic epitopes and protease inhibitors) can be prepared in water or saline, and optionally mixed with a nontoxic surfactant. Compositions for intravenous or intra-arterial administration may include sterile aqueous solutions that may also contain buffers, liposomes, diluents and other suitable additives.

Oils useful in parenteral compositions include petroleum, animal, vegetable, or synthetic oils. Specific examples of oils useful in such compositions include peanut, soybean, sesame, cottonseed, corn, olive, petrolatum, and mineral. Suitable fatty acids for use in parenteral compositions include oleic acid, stearic acid, and isostearic acid. Ethyl oleate and isopropyl myristate are examples of suitable fatty acid esters.

Suitable soaps for use in parenteral compositions include fatty alkali metal, ammonium, and triethanolamine salts, and suitable detergents include (a) cationic detergents such as, for example, dimethyl dialkyl ammonium halides, and alkyl pyridinium halides; (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-.beta.-aminopropionates, and 2-alkyl-imidazoline quaternary ammonium salts, and (e) mixtures thereof.

The parenteral compositions typically will contain from about 0.5 to about 25% by weight of the active ingredient in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such compositions will typically range from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral compositions can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

The pharmaceutical dosage forms suitable for injection or infusion can include sterile aqueous solutions or dispersions comprising the active ingredient that are adapted for administration by encapsulation in liposomes. In all cases, the ultimate dosage form must be sterile, fluid and stable under the conditions of manufacture and storage.

Sterile injectable solutions are prepared by incorporating ghrelin or a ghrelin-like compound or pharmaceutically acceptable salt thereof in the required amount in the appropriate solvent with various of the other ingredients enumerated above, as required, followed by filter sterilization.

Compositions for Topical Administration

The compounds of the invention can also be delivered topically. Regions for topical administration include the skin surface and also mucous membrane tissues of the rectum, nose, mouth, and throat. Compositions for topical administration via the skin and mucous membranes should not give rise to signs of irritation, such as swelling or redness.

The topical composition may include a pharmaceutically acceptable carrier adapted for topical administration. Thus, the composition may take the form of a suspension, solution, ointment, lotion, cream, foam, aerosol, spray, suppository, implant, inhalant, tablet, capsule, dry powder, syrup, balm or lozenge, for example. Methods for preparing such compositions are well known in the pharmaceutical industry.

The compounds of the present invention may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents. Compositions suitable for topical administration in the mouth include lozenges comprising active agents in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Creams, ointments or pastes according to the present invention are semi-solid compositions of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as steric or oleic acid together with an alcohol such as propylene glycol or a macrogel. The composition may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

The pharmaceutical agent-chemical modifier complexes described herein can be administered transdermally. Transdermal administration typically involves the delivery of a pharmaceutical agent for percutaneous passage of the drug into the systemic circulation of the patient. The skin sites include anatomic regions for transdermally administering the drug and include the forearm, abdomen, chest, back, buttock, mastoidal area, and the like.

Transdermal delivery is accomplished by exposing a source of the complex to a patient's skin for an extended period of time. Transdermal patches have the added advantage of providing controlled delivery of a pharmaceutical agent-chemical modifier complex to the body. See Transdermal Drug Delivery: Developmental Issues and Research Initiatives, Hadgraft and Guy (eds.), Marcel Dekker, Inc., (1989); Controlled Drug Delivery: Fundamentals and Applications, Robinson and Lee (eds.), Marcel Dekker Inc., (1987); and Transdermal Delivery of Drugs, Vols. 1-3, Kydonieus and Berner (eds.), CRC Press, (1987). Such dosage forms can be made by dissolving, dispersing, or otherwise incorporating the pharmaceutical agent-chemical modifier complex in a proper medium, such as an elastomeric matrix material. Absorption enhancers can also be used to increase the flux of the compound across the skin. The rate of such flux can be controlled by either providing a rate-controlling membrane or dispersing the compound in a polymer matrix or gel.

A variety of types of transdermal patches will find use in the methods described herein. For example, a simple adhesive patch can be prepared from a backing material and an acrylate adhesive. The pharmaceutical agent-chemical modifier complex and any enhancer are formulated into the adhesive casting solution and allowed to mix thoroughly. The solution is cast directly onto the backing material and the casting solvent is evaporated in an oven, leaving an adhesive film. The release liner can be attached to complete the system.

Alternatively, a polyurethane matrix patch can be employed to deliver the pharmaceutical agent-chemical modifier complex. The layers of this patch comprise a backing, a polyurethane drug/enhancer matrix, a membrane, an adhesive, and a release liner. The polyurethane matrix is prepared using a room temperature curing polyurethane prepolymer. Addition of water, alcohol, and complex to the prepolymer results in the formation of a tacky firm elastomer that can be directly cast only the backing material.

A further embodiment of this invention will utilize a hydrogel matrix patch. Typically, the hydrogel matrix will comprise alcohol, water, drug, and several hydrophilic polymers. This hydrogel matrix can be incorporated into a transdermal patch between the backing and the adhesive layer.

The liquid reservoir patch will also find use in the methods described herein. This patch comprises an impermeable or semipermeable, heat sealable backing material, a heat sealable membrane, an acrylate based pressure sensitive skin adhesive, and a siliconized release liner. The backing is heat sealed to the membrane to form a reservoir which can then be filled with a solution of the complex, enhancers, gelling agent, and other excipients.

Foam matrix patches are similar in design and components to the liquid reservoir system, except that the gelled pharmaceutical agent-chemical modifier solution is constrained in a thin foam layer, typically a polyurethane. This foam layer is situated between the backing and the membrane which have been heat sealed at the periphery of the patch.

For passive delivery systems, the rate of release is typically controlled by a membrane placed between the reservoir and the skin, by diffusion from a monolithic device, or by the skin itself serving as a rate-controlling barrier in the delivery system. See U.S. Pat. Nos. 4,816,258; 4,927,408; 4,904,475; 4,588,580, 4,788,062; and the like. The rate of drug delivery will be dependent, in part, upon the nature of the membrane. For example, the rate of drug delivery across membranes within the body is generally higher than across dermal barriers. The rate at which the complex is delivered from the device to the membrane is most advantageously controlled by the use of rate-limiting membranes which are placed between the reservoir and the skin. Assuming that the skin is sufficiently permeable to the complex (i.e., absorption through the skin is greater than the rate of passage through the membrane), the membrane will serve to control the dosage rate experienced by the patient.

Suitable permeable membrane materials may be selected based on the desired degree of permeability, the nature of the complex, and the mechanical considerations related to constructing the device. Exemplary permeable membrane materials include a wide variety of natural and synthetic polymers, such as polydimethylsiloxanes (silicone rubbers), ethylenevinylacetate copolymer (EVA), polyurethanes, polyurethane-polyether copolymers, polyethylenes, polyamides, polyvinylchlorides (PVC), polypropylenes, polycarbonates, polytetrafluoroethylenes (PTFE), cellulosic materials, e.g., cellulose triacetate and cellulose nitrate/acetate, and hydrogels, e.g., 2-hydroxyethylmethacrylate (HEMA).

Other items may be contained in the device, such as other conventional components of therapeutic products, depending upon the desired device characteristics. For example, the compositions according to this invention may also include one or more preservatives or bacteriostatic agents, e.g., methyl hydroxybenzoate, propyl hydroxybenzoate, chlorocresol, benzalkonium chlorides, and the like. These pharmaceutical compositions also can contain other active ingredients such as antimicrobial agents, particularly antibiotics, anesthetics, analgesics, and antipruritic agents.

Compositions for Administration as Suppositories

The compounds of the present invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The active compound may be formulated into a suppository comprising, for example, about 0.5% to about 50% of a compound of the invention, disposed in a polyethylene glycol (PEG) carrier (e.g., PEG 1000 [96%] and PEG 4000 [4%].

Formulation

In a preferred aspect the present invention contemplates pharmaceutical compositions useful for practicing the therapeutic methods described herein. Pharmaceutical compositions of the present invention contain a physiologically tolerable carrier together with at least one species of a secretagogue, such as ghrelin or a ghrelin-like compound as described herein, dissolved or dispersed therein as an active ingredient. In a preferred embodiment, the pharmaceutical composition is not immunogenic when administered to a human individual for therapeutic purposes, unless that purpose is to induce an immune response.

In one aspect the invention relates to a pharmaceutical composition comprising at least one secretagogue, such as ghrelin or a ghrelin-like compound as defined above in formula 1. In a preferred embodiment the pharmaceutical composition comprises at least two different ghrelin-like compounds as defined above in formula I in order to increase the effect of the treatment. The difference may for example be compounds having different acylations as discussed above.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects such as nausea, dizziness, gastric upset and the like.

The preparation of a pharmacological composition that contains active ingredients dissolved or dispersed therein is well understood in the art. Typically such compositions are prepared as sterile injectables either as liquid solutions or suspensions, aqueous or non-aqueous, however, solid forms suitable for solution, or suspensions, in liquid prior to use can also be prepared. The preparation can also be emulsified.

The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. It is preferred that the formulation has a pH within the range of 3.5-8, such as in the range 4.5-7.5, such as in the range 5.5-7, such as in the range 6-7.5, most preferably around 7.3. However, as is understood by one skilled in the art, the pH range may be adjusted according to the individual treated and the administration procedure. For example, certain secretagogues, such as ghrelin and ghrelin homologs, may be easily stabilised at a lower pH, so in another preferred embodiment of the the invention the formulation has a pH within the range 3.5-7, such as 4-6, such as 5-6, such as 5.3-5.7, such as 5.5.

The pharmaceutical composition of the present invention can include pharmaceutically acceptable salts of the compounds therein. These salts will be ones which are acceptable in their application to a pharmaceutical use. By that it is meant that the salt will retain the biological activity of the parent compound and the salt will not have untoward or deleterious effects in its application and use in treating diseases.

Pharmaceutically acceptable salts are prepared in a standard manner. If the parent compound is a base it is treated with an excess of an organic or inorganic acid in a suitable solvent. If the parent compound is an acid, it is treated with an inorganic or organic base in a suitable solvent.

The compounds of the invention may be administered in the form of an alkali metal or earth alkali metal salt thereof, concurrently, simultaneously, or together with a pharmaceutically acceptable carrier or diluent, especially and preferably in the form of a pharmaceutical composition thereof, whether by e.g. oral, rectal, or parenteral (including subcutaneous) route, in an effective amount.

Examples of pharmaceutically acceptable acid addition salts for use in the present inventive pharmaceutical composition include those derived from mineral acids, such as hydrochloric, hydrobromic, phosphoric, metaphosphoric, nitric and sulfuric acids, and organic acids, such as tartaric, acetic, citric, malic, lactic, fumaric, benzoic, glycolic, gluconic, succinic, p-toluenesulphonic acids, and arylsulphonic, for example.

Other suitable pharmaceutically acceptable salts include the acid addition salts (formed with the free amino groups of the polypeptide). Other examples of salts include include pharmaceutically acceptable acid addition salts, pharmaceutically acceptable metal salts, ammonium salts and alkylated ammonium salts. Acid addition salts include salts of inorganic acids as well as organic acids. Representative examples of suitable inorganic acids include hydrochloric, hydrobromic, hydriodic, phosphoric, sulpfuric and nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, ethylenediaminetetraacetic (EDTA), p-aminobenzoic, glutamic, benzenesulfonic and ptoluenesulfonic acids and the like. Further examples of pharmaceutically acceptable inorganic or organic acid addition salts include the pharmaceutical acceptable salts listed in J. Pharm. Sci. 1977,66,2, which is incorporated herein by reference. Examples of metal salts include lithium, sodium, potassium and magnesium salts and the like.

Examples of ammonium and alkylated ammonium salts include ammonium, methylammonium, dimethylammonium, trimethylammonium, ethylammonium, hydroxyethylammonium, diethylammonium, butylammonium and tetramethylammonium salts and the like.

Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Also included within the scope of compounds or pharmaceutical acceptable acid addition salts thereof in the context of the present invention are any hydrates (hydrated forms) thereof.

For parenteral administration, solutions of the present compounds in sterile aqueous solution, aqueous propylene glycol or sesame or peanut oil may be employed. Such aqueous solutions should be suitably buffered if necessary, and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Liquid compositions can also contain liquid phases in addition to and to the exclusion of water. Exemplary of such additional liquid phases are glycerin, vegetable oils such as cottonseed oil, organic esters such as ethyl oleate, and water-oil emulsions.

Suitable pharmaceutical carriers include inert solid diluents or fillers, sterile aqueous solution and various organic solvents. Examples of solid carriers are lactose, terra alba, sucrose, cyclodextrin, talc, gelatine, agar, pectin, acacia, magnesium stearate, stearic acid or lower alkyl ethers of cellulose. Examples of liquid carriers are syrup, peanut oil, olive oil, phospholipids, fatty acids, fatty acid amines, polyoxyethylene or water. Administered by nasal aerosol or inhalation formulations may be prepared, for example, as solutions in saline, employing benzyl alcohol or other suitable preservatives, absorption promoters to enhance bioavailability, employing fluorocarbons, and/or employing other solubilizing or dispersing agents.

The pharmaceutical compositions formed by combining the compounds of the invention and the pharmaceutical acceptable carriers are then readily administered in a variety of dosage forms suitable for the disclosed routes of administration. The formulations may conveniently be presented in unit dosage form by methods known in the art of pharmacy.

In a preferred embodiment of the invention the formulation comprises the secretagogue or a salt thereof as a lyophilisate and the formulation further comprises a solvent, said lyophilisate and said solvent being in separate compartements until administration.

In another embodiment the formulation is a solution of the secretagogue or a salt thereof.

In both embodiment the solvent may be any suitable solvents, such as described herein, and preferably the solvent is saline.

The invention also relates to a method for preparing a medicament or pharmaceutical composition comprising a compound of the invention, comprising admixing at least one ghrelin-like compound, as defined above in formula I, with a physiologically acceptable carrier.

In a still further aspect, the invention relates to a pharmaceutical composition comprising, as an active ingredient, a compound as defined above in formula I or a pharmaceutically acceptable salt thereof together with a pharmaceutically-acceptable carrier.

Accordingly, the formulation may further include the transport molecules as described above.

Combination Treatments

In a further aspect of the invention the present compounds may be administered in combination with further pharmacologically active substances or therapeutic method or other pharmacologically active material, By the phrase “in combination” with another substance(s) and/or therapeutic method(s) is meant herein that said another substance(s) and/or therapeutic method(s) is administered to the individual thus treated before, during (including concurrently with) and/or after treatment of an individual with a secretagogue. In all cases of combination treatment described herein, the combination may be in the form of kit-in-part systems, wherein the combined active substances may be used for simultaneous, sequential or separate administration. In all cases, it is preferred that any of the herein-mentioned medicaments are administered in pharmaceutically effective amounts, i.e. an administration involving a total amount of each active component of the medicament or pharmaceutical composition or method that is sufficient to show a meaningful patient benefit.

In the following sections, combination therapies for use in preferred embodiments of the present invention are grouped as follows:
1) Combinations wherein all active ingredients are appetite-regulating agents or in other ways useful for treating cachexia
2) Combinations of the secretagogue, such as ghrelin or a ghrelin-like compound, with an ingredient or therapy active against a disease causing or being associated with the disease or condition treated with the secretagogue, such as ghrelin or a ghrelin-like compound.
3) Combinations of the secretagogue, such as ghrelin or a ghrelin-like compound, with an ingredient active or therapy against symptoms associated with the disease or condition treated with the secretagogue, such as ghrelin or a ghrelin-like compound.

Of course, combinations of the above groups are also within the scope of this invention.

Combinations wherein all active ingredients are appetite-regulating agents or in other ways useful for treating cachexia and/or lipodystrophy

The secretagogue(s) according to the invention can be administered in combination with other appetite-regulating agents, including more than one type of growth hormone secretagogue, such as another ghrelin-like compound, such as a ghrelin-like compound comprising a structure defined by formula I, described herein. Other secretagogues suitable for combination administration with another secretagogue compound are any of the secretagogue compounds described herein. In one preferred embodiment of the present invention, wild type ghrelin (most preferably human wild type ghrelin) is administered in combination with a different, ghrelin-like compound—this combination is envisaged to enhance and/or prolong the effect of the secretagogues on the ghrelin receptor. In another preferred embodiment of the present invention, a ghrelin-like compound that is not wild type ghrelin is administered in combination with a different ghrelin-like compound that is not wild-type ghrelin—again, this combination is envisaged to enhance and/or prolong the effect of the secretagogues on the ghrelin receptor. In a similar way, several different secretagogues may be administered to an individual to increase efficacy on the ghrelin receptor—such as greater than 2 different secretagogue types, such as 3, such as 4, such as 5, such as 6, such as 7, such as greater than 8 different secretagogue types. The secretagogue according to the invention, such as ghrelin or a ghrelin-like compound(s) can also be administered in combination with a pharmaceutically effective amount of a growth hormone, including hGH.

In one preferred embodiment of the present invention the secretagogue, such as ghrelin or a ghrelin-like compound, may be administered in combination with IGF-1, IGFBP-3, or ALS, preferably with IGF-1. The rationale behind this combination treatment is to increase the level of IGF-1, IGFBP-3, and/or ALS found to be low in cachectic individuals.

In a further embodiment of the invention, the secretagogues, such as ghrelin or a ghrelin-like compound, may be administered in combination with compounds known to stimulate appetite, such as melanocortin receptor antagonists, neuropeptide Y receptor agonists including agonists selective for individual subtypes of the neuropeptide Y receptors, leptin or leptin receptor agonists, cannabinoids including marijuana and marijuana derivatives, antipsychotics, especially atypical antipsychotics such as sertindole, Sulpirid, Clozapine, Risperidone, Quetiapin, Amisulpride, Ziprasidon, and Olanzapine.

Combinations of the secretagogue, such as ghrelin or a ghrelin-like compound, with an ingredient or therapy active against a disease causing or being associated with the disease or condition treated with the secretagogue, such as ghrelin or a ghrelin-like compound.

In particular in relation to cancer cachexia, administration of a secretagogue, such as a ghrelin-like compound, may be used in combination with any anti-cancer therapy, including antineoplastic chemotherapy, radiotherapy and surgical treatment. In particular it is used in combination with chemotherapy and radiotherapy. Thus, in one embodiment the present invention relates to a method of treating cancer comprising administering an effective amount of radiotherapy and an effective amount of a secretagogue, such as a ghrelin-like compound according to the invention. The treatment with the secretagogue, such as a ghrelin-like compound, may be started before the radiotherapy treatment initiates. It may be administered continuously during the radiotherapy or it may be administered at intervals, for example between periods with radiotherapy therapy.

In another embodiment the present invention relates to a method of treating cancer comprising administering an effective amount of antineoplastic chemotherapy and an effective amount of a secretagogue, such as a ghrelin-like compound according to the invention. The treatment with the secretagogue, such as a ghrelin-like compound, may be started before the chemotherapy treatment initiates. It may be administered continuously during the chemotherapy or it may be administered at intervals, for example between periods with chemotherapy therapy.

Furthermore, the combination treatment may be co-formulations of the secretaogue, such as a ghrelin-like compound, and the antineoplastic chemotherapy.

The secretagogue according to the invention, such as ghrelin or a ghrelin-like compound(s), may also be administered in combination with a pharmaceutically effective amount of glucocorticoid steroids and prokinetic treatment as well as other treatment used in cancer therapy. Thus, in another preferred embodiment of the present invention, the secretagogue according to the invention, such as ghrelin or a ghrelin-like compound(s), is administered in combination with a pharmaceutically effective amount of one or more of:

progestational drugs, such as megastrol and/or

cyproheptadines (and/or other 5-HT receptor antagonists), and/or

branched chain amino acids, and/or

oxandralin and/or

anti-TNF-alpha agents, such as infliximab, etanercept, or adalimumab and/or

testosterone and/or

“cocktail” comprising immunonutrition, antioxidants and COX-2 inhibitors and/or

cannabinoids, and/or

eicosapentaenoic acid and/or

melatonin and/or

thalidomide and/or

a β₂ adrenergic drug; most preferably for the treatment of cachexia, such as cancer cachexia.

In yet another embodiment the secretagogue, such as a ghrelin-like compound, is administered in combination with anti-inflammatory compounds, preferably an NSAID, such as indomethacin, and COX1 inhibitors or COX2 inhibitors, and/or anti-TNF-alpha compounds such as infliximab, etanercept, or adalimumab. Another combination may be with erythropoietin/EPO. Another combination can be with angiotensin II lowering agents, such as Vitor. Another combination can be with selective androgen receptor modulator(s). Another combination may be with one or more of leptin, agonists of the renin-angiotensin system, opioid receptor agonists or peroxisome proliferator-activated receptor gamma agonists.

In relation to treatment of lipodystrophy, the invention relates in another embodiment, to a treatment wherein a secretagogue, such as ghrelin, more preferably a ghrelin-like compound, is administered in combination with a lipodystrophy treatment, such as one ore more of the treatments or compounds described herein suitable for treating a lipodystrophic syndrome.

Thus, other pharmacologically active substances that may be administered in combination with said secretagogue, such as a ghrelin-like compound, in the methods of the present invention comprise:
a) Leptin: leptin has been shown to have a positive effect on the metabolic abnormalities associated with lipodystrophy. This treatment has proven to be beneficial both to those patients that suffer from a low plasma level of leptin and to those that have a normal level.
b) Peroxisome proliferator-activated receptor (PPAR-γ) agoinsts: PPAR-γ has in several studies been demonstrated to be important for adipocyte metabolism and metabolic syndrome and it is proposed that PPAR-γ agonists will decrease the symptoms of lipodystrophy.
c) Agonists of the renin-angiotensin system: it has been shown that treatment with HAART increases the activity of ACE in the T-cells, which means that agonists of the rennin-angiotensin system may improve HMRT induced lipodystrophy.
d) Opioid receptor antagonists: opioid receptor antagonists, such as Naloxone and Naltrexone, have been shown to prolong the period of time from protease inhibitor treatment to development of the first symptoms of lipodystrophy.
e) Des-acyl ghrelin: ghrelin in combination with des-acyl ghrelin have been found to decrease insulin resistance, which is an important feature of the lipodystrophy syndrome.
f) Adiponectin and anti-diabetic treatment including other compounds for the treatment and/or prevention of insulin resistance and diseases wherein insulin resistance is the pathophysiological mechanism.
g) Therapy with rhGH has been reported to cause reduction in the size of ‘buffalo hump’, truncal fat and to increase the lean body mass in a small number of patients, However, fat loss and lipid abnormalities did not improve and blood glucose control worsened. Examples of syndromes treated with hGH include HIV, AIDS and cancer. Without being bound by theory, it is believed that treatment with ghrelin or a analog thereof would maintain and/or increase body fat in patients being treated with hGH, thereby effectively counteracting or at least reducing lipodystrophy caused by hGH. Thus, in one preferred embodiment, the present invention relates to use of ghrelin or an analog thereof in combination with a growth hormone, preferably in individuals suffering from HIV or AIDS and/or cancer cachexia. Said treatment with ghrelin or an analog thereof may be prior to, and/or during and/or after the individual is subjected to treatment with a growth hormone. Said growth hormone is preferably hGH.
h) Treatment with combinations of different secretagogues as described above under 1).

Combinations of the secretagogue, such as ghrelin or a ghrelin-like compound, with an ingredient active or therapy against symptoms associated with the disease or condition treated with the secretagogue, such as ghrelin or a ghrelin-like compound.

The invention further relates to combination treatment, wherein one of the ingredients in the combination is used for treating symptoms or conditions that may be encountered in individuals suffering from cachexia. Thus, uses and combination treatments involving administration of a secretagogue, such as the ghrelin-like compound according to the present invention, can also involve treatment in combination with one or more of

a) prophylaxis and/or alleviation and/or treatment of a clinical depression, which combination treatment further comprises administering an antidepressant, a prodrug thereof, or a pharmaceutically acceptable salt of said antidepressant or said prodrug. In the above combination treatment, the antidepressant is preferably a norepinephrine reuptake inhibitor (NERI), a selective serotonin reuptake inhibitor (SSRI), a monoamine oxidase inhibitor (MAO), a combined NERI/SSRI, or an atypical antidepressant, a prodrug of said antidepressant or a pharmaceutically acceptable salt of said antidepressant or said prodrug.

One preferred antidepressant is a selective serotonin reuptake inhibitor (SSRI), a prodrug thereof or a pharmaceutically acceptable salt of said SSRI or said prodrug. The SSRI is preferably citalopram, escitalopram, femoxetine, fluoxetine, fluvoxamine, indalpine, indeloxazine, milnacipran, paroxetine, sertraline, sibutramine or zimeldine, a prodrug of said SSRI or a pharmaceutically acceptable salt of said SSRI or said prodrug. Of the above, citalopram and escitalopram, a prodrug or a pharmaceutically acceptable salt thereof, are preferred in certain embodiments of combination treatments according to the present invention.

b) prophylaxis and/or alleviation and/or treatment of an emetic condition, including nausia and vomiting, which combination treatment further comprises administering an antiemetic agent, a prodrug thereof, or a pharmaceutically acceptable salt of said antiemetic agent or said prodrug. Preferred antiemetic agents used in combination treatments according to the invention include meclizine hydrochloride, prochlorperazine, promethazine, trimethobenzamide hydrochloride and ondansetron hydrochloride.

In particular emesis may be caused by cancer, either due to the anti-cancer treatment or due to the cancer disease as such.

c) prophylaxis and/or alleviation and/or treatment of a psychotic condition, which combination treatment further comprises administering an antipsychotic agent, a prodrug thereof or a pharmaceutically acceptable salt of said antipsychotic agent or said prodrug Preferred antipsychotic agents used in combination treatments in accordance with the present invention include chlorpromazine, haloperidol, clozapine, loxapine, molindone hydrochloride, thiothixene, olanzapine, ziprasidone, ziprasidone hydrochloride, prochlorperazine, perphenazine, trifluoperazine hydrochloride and risperidone.

d) prophylaxis and/or alleviation and/or treatment of anxiety, which combination treatment further comprises administering an antianxiety agent, a prodrug thereof or a pharmaceutically acceptable salt of said antianxiety agent or said prodrug. Preferred antianxiety agents used in combination treatments in accordance with the invention include alprazolam, clonazepam, lorazepam, oxazepam, chlordiazepoxide hydrochloride, diazepam, buspirone hydrochloride, doxepin hydrochloride, hydroxyzine pamoate and clonazepam.

Medical Packaging

The compounds of the invention may be administered alone or in combination with pharmaceutically acceptable carriers or excipients, in either single or multiple doses. The formulations may conveniently be presented in unit dosage form by methods known to those skilled in the art.

It is preferred that the compounds according to the invention is provided in a kit. Such a kit typically contains an active compound in dosage forms for administration. A dosage form contains a sufficient amount of active compound such that a desirable effect can be obtained when administered to a subject, preferably prior to at least one meal a day, more preferably prior to each main meal, such as three times a day, during the course of 1 or more days.

Thus, it is preferred that the medical packaging comprises an amount of dosage units corresponding to the relevant dosage regimen. Accordingly, in one embodiment, the medical packaging comprises a pharmaceutical composition comprising a compound as defined above or a pharmaceutically acceptable salt thereof and pharmaceutically acceptable carriers, vehicles and/or excipients, said packaging having from 7 to 21 dosage units, or multipla thereof, thereby having dosage units for one week of administration or several weeks of administration.

In one embodiment the medical packaging is for administration once daily in a week, and comprises 7 dosage units, in another embodiment the medical packaging is for administration twice daily, and comprises 14 dosage units. In yet another more preferred embodiment the medical packaging is for administration three times daily, and comprises 21 dosage units.

The dosage units are as defined above, i.e. a dosage unit preferably comprises an amount of the ghrelin-like compound or a salt thereof equivalent to from 0.3 μg to 600 mg ghrelin, such as of from 2.0 μg to 200 mg ghrelin, such as from 5.0 μg to 100 mg ghrelin, such as from 10 μg to 50 mg ghrelin, such as from 10 μg to 5 mg ghrelin, such as from 10 μg to 1.0 mg ghrelin.

The medical packaging may be in any suitable form for parenteral, in particular subcutaneous administration. In a preferred embodiment the packaging is in the form of a cartridge, such as a cartridge for an injection pen, the injection pen being such as an injection pen known from insulin treatment.

When the medical packaging comprises more than one dosage unit, it is preferred that the medical packaging is provided with a mechanism to adjust each administration to one dosage unit only.

Preferably, a kit contains instructions indicating the use of the dosage form to achieve a desirable affect and the amount of dosage form to be taken over a specified time period. Accordingly, in one embodiment the medical packaging comprises instructions for administering the pharmaceutical composition. In particular said instructions may include instructions referring to administration of said pharmaceutical composition either during a meal, or preferably at the most 45 minutes prior to a meal, such as at the most 30 minutes prior to a meal, such as at the most 25 minutes prior to a meal, such as at the most 20 minutes prior to a meal, such as at the most 15 minutes prior to a meal, such as at the most 10 minutes prior to a meal, such as at the most 5 minutes prior to a meal.

Method for Monitoring the Effect of Treatment with Ghrelin and/or a Ghrelin-like Compound

In another aspect, the present invention relates to a method for monitoring the effect of the administration of a secretagogue, such as the ghrelin-like compounds of the present invention, in a methods of the present invention, comprising measuring one or more markers, in particular markers selected from IGF-I, IGFBP-3, ALS (acidic labled), thyroid hormones, sex hormones, and albumin, more preferably from IGF-I, IGFBP-3, ALS (acidic labled), more preferably IGF-1. These markers are all low in cachetic patients and are expected to increase after treatment with ghrelin. The present invention thus relates to a method for monitoring the effect of any of the treatments of an individual with a secretagogue described herein, said method comprising measuring the blood level of said individual of one or more of:

(i) IGF-1 and/or

(ii) IGFBP-3 and/or

(iii) ALS and/or

(iv) one or more thyroid hormones and/or

(v) one or more sex hormones and/or

(vi) albumin

or, more preferably, one or more of:

(i) IGF-1 and/or

(ii) IGFBP-3 and/or

(iii) ALS.

Methods for measuring substances in the blood level of an individual are well known in the art. As an example, an isolated blood sample may be tested by methods such as Western blot or by enzyme-linked assay (ELISA).

### DETAILED DESCRIPTION OF DRAWINGS

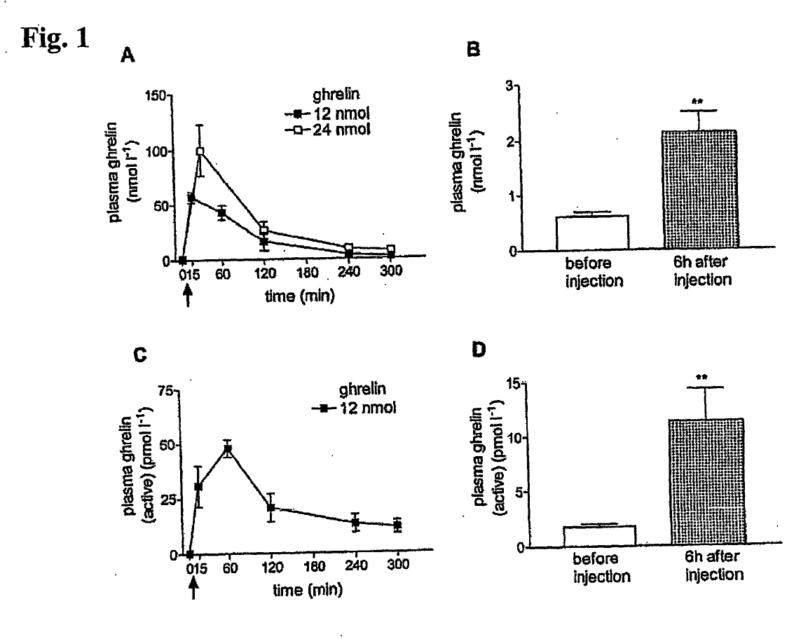

FIGS. 1A-D: A) Subcutaneous injection (arrows) of 12 and 24 nmol of ghrelin to mice raised the plasma ghrelin concentration (n=6 in each group).

B) Plasma ghrelin was elevated still 6 h after injection of 12 nmol of ghrelin.

C) Plasma concentration of active (octanoylated) ghrelin was greatly elevated in response to 12 nmol of ghrelin and

D) remained high 6 h after injection. Means±S.E.M (n=4).

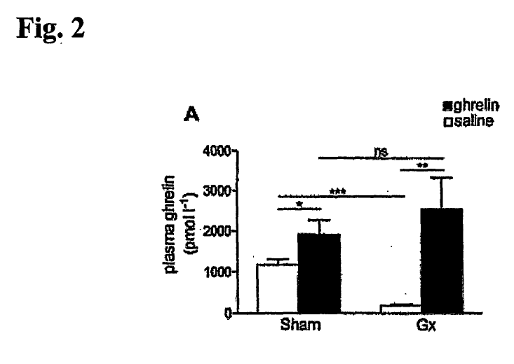

FIG. 2: Plasma concentration of ghrelin in mice 8 weeks after sham operation or gastrectomy with or without the daily administration of ghrelin (12 nmol sc). Gastrectomy reduced the plasma ghrelin concentration. The plasma ghrelin concentration was elevated 16-18 h after injection of ghrelin. Means±S.E.M. (n=10).

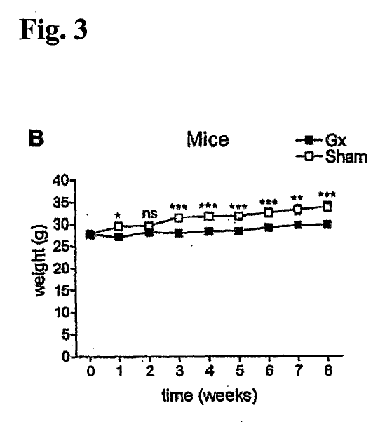

FIG. 3: Plasma concentration of ghrelin in mice 8 weeks after sham operation or gastrectomy with or without the daily administration of ghrelin (12 nmol sc). Gastrectomy reduced the plasma ghrelin concentration. The plasma ghrelin concentration was elevated 16-18 h after injection of ghrelin. Means±S.E.M. (n=10).

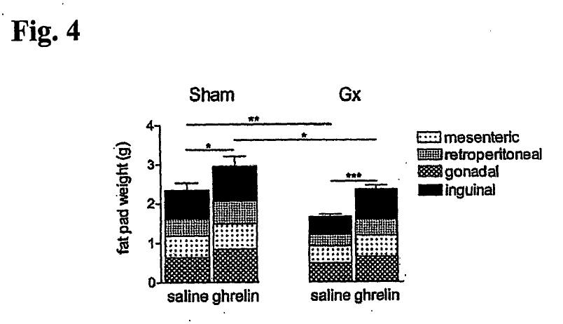

FIG. 4: Effects of gastrectomy and/or daily administration of ghrelin (12 nmol sc) on fat depots in mice. The duration of the study was 8 weeks. Gastrectomy reduced the amount of fat and ghrelin administration normalized the fat depots. Administration of ghrelin increased the amount of fat in sham-operated rats. Means±S.E.M. (n=10-12).

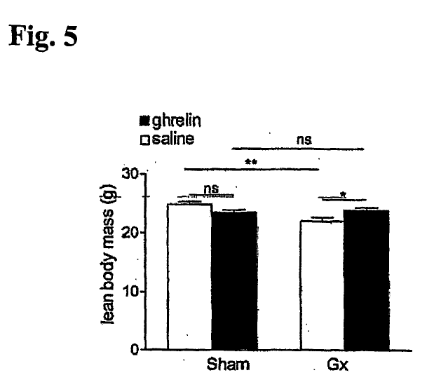

FIG. 5: Lean body mass in mice subjected to sham operation or gastrectomy with or without the daily administration of ghrelin (12 nmol sc). Gastrectomy reduced the lean body mass and administration of ghrelin reversed the effect. There was no difference in lean body mass between the two groups of sham-operated mice, receiving either saline or ghrelin. *p<0.05, **p<0.01. Means±S.E.M. (n=10-12).

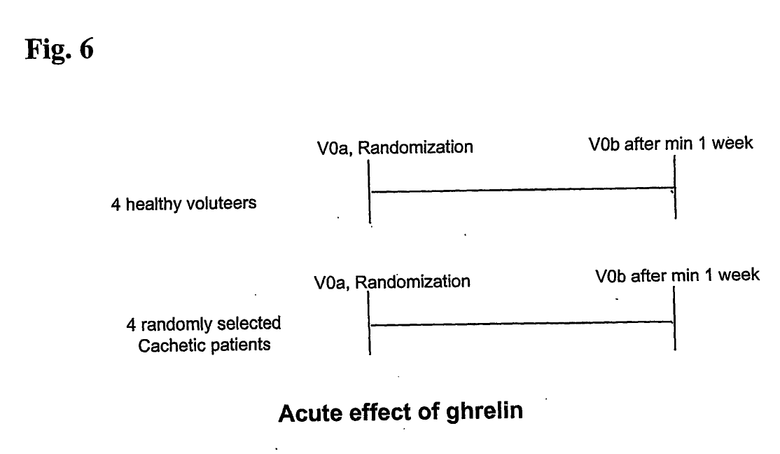

FIG. 6: graphical overview of clinical trial to evaluate acute effects of ghrelin administration in four healthy volunteers and in 4 cancer cachetic patients. V0a and V0b represent visits during which patients are treated with the relevant test or control compound.

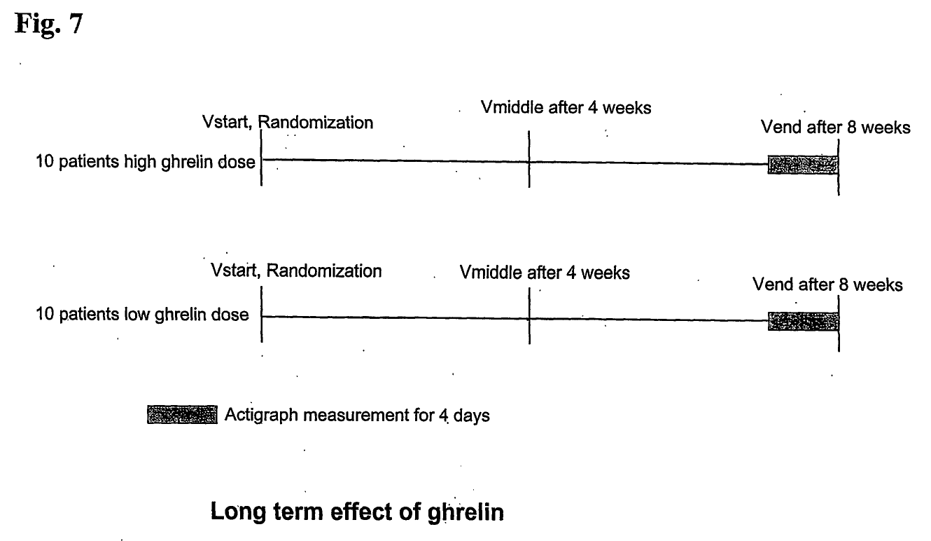

FIG. 7: graphical overview of clinical trial to evaluate long term effects of low dose or high dose ghrelin administration in patients suffering from cancer cachexia.

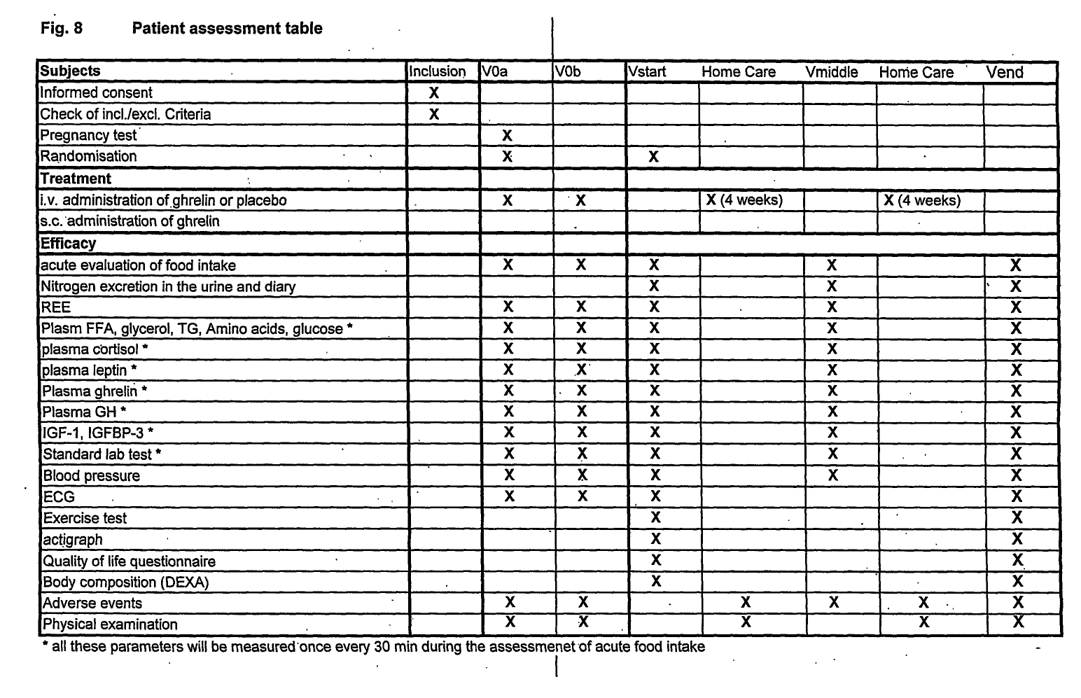

FIG. 8: Patient assessment table for use in evaluating patient condition in the clinical trials described in Example 11.

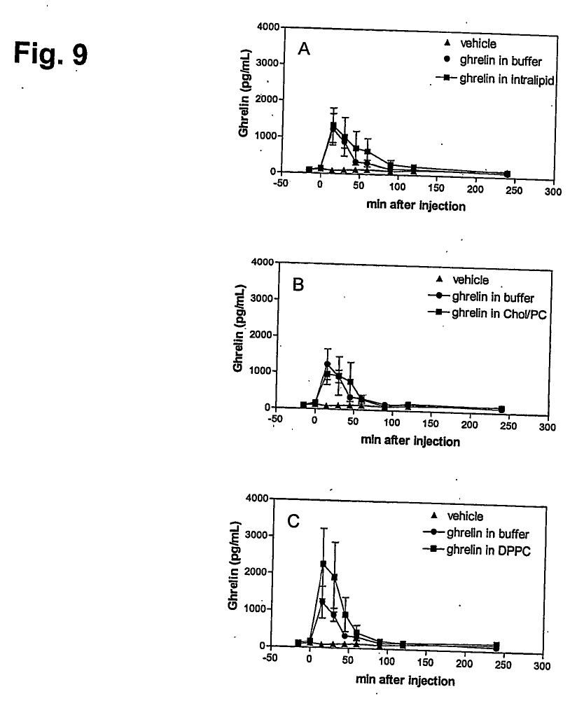

FIG. 9A-C: Figures demonstrating the pharmacokinetics of different ghrelin formulations in rat (see description in examples 9 and 10).

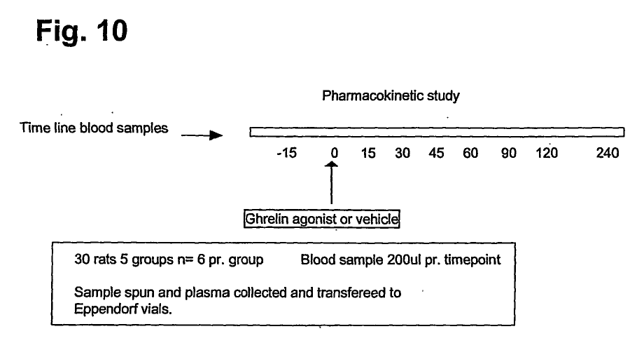

FIG. 10: Overview of animal study to investigate pharmacokinetics of ghrelin formulations (see further description in examples 9 and 10).

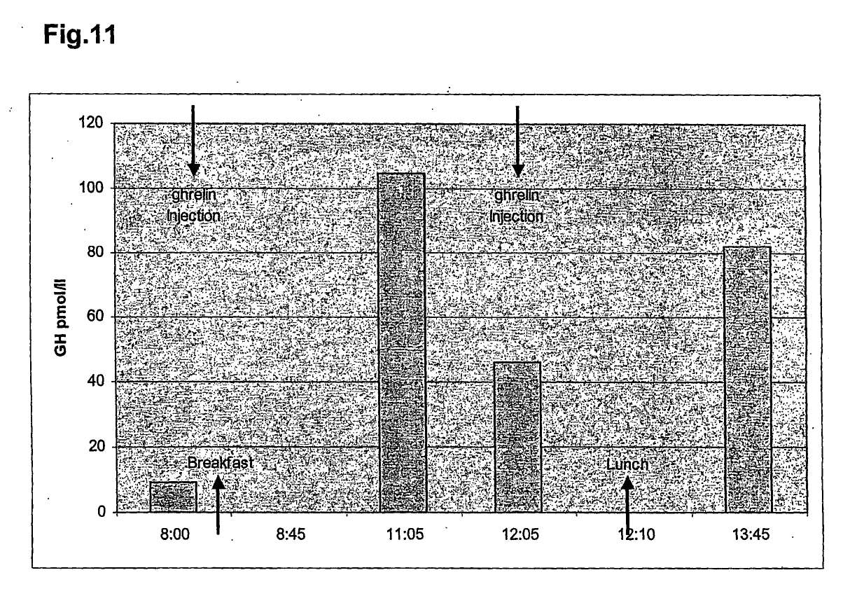

FIG. 11: Graph showing effect of subcutaneous ghrelin administration on growth hormone levels. The results show a clear increase in growth hormone levels of more than 5 fold.

### EXAMPLES

### Example 1

Competition Binding Assays

Transfected COS-7 cells were transferred to culture plates one day after transfection at a density of 1×10⁵ cells per well aiming at 5-8% binding of the radioactive ligand. Two days after transfection competition binding experiments were performed for 3 hours at 4° C. using 25 pM of ¹²⁵I-ghrelin (Amersham, Little Chalfont, UK). Binding assays were performed in 0.5 ml of a 50 mM Hepes buffer, pH 7.4, supplemented with 1 mM CaCl₂, 5 mM MgCl₂, and 0.1% (w/v) bovine serum albumin, 40 microgram/ml bacitracin. Non-specific binding was determined as the binding in the presence of 1 micromole of unlabeled ghrelin. Cells were washed twice in 0.5 ml of ice-cold buffer and. 0.5-1 ml of lysis buffer (8 M Urea, 2% NP40 in 3 M acetic acid) was added and the bound radioactivity was counted. Determinations were made in duplicate. Initial experiments showed that steady state binding was reached with the radioactive ligand under these conditions.

### Example 2

Synthetic Production of Ghrelin-Like Compound

Amino acid derivatives and synthesis reagents, can be obtained from commercial sources. Peptide chain extension can be performed using Applied Biosystem 433A synthesizer produced by Perkin Elmer, and a protected peptide derivative-resin can be constructed by the Boc or Fmoc method. The protected peptide resin obtained by the Boc method is deprotected with anhydrous hydrogen fluoride (HF) in the presence of p-cresol thereby releasing the peptide, which is then purified. The protected peptide resin obtained by the Fmoc method is deprotected with trifluoroacetic acid (TFA) or dilute TFA containing various scavengers, and the released peptide is purified. Purification is performed in reversed phase HPLC on a C4 or C18 column. The purity of the purified product can be confirmed by reverse phase HPLC, and its structure can be confirmed by amino acid composition analysis and mass spectrometry.

The peptide of the present invention can be produced by a conventional peptide synthesis method. Specifically, synthesis of acylated or alkylated peptides is exemplified below. Further, human-derived ghrelin (which may be abbreviated hereinafter to hGhrelin) or rat-derived ghrelin (which may be abbreviated hereinafter to rGhrelin) is reacted with trypsin or chymotrypsin or both the enzymes successively to give the following ghrelin fragments: 19. Ghrelin (16-28), 20. hGhrelin (1-15), 21. rGhrelin (1-15), 23. hGhrelin (1-11), 24. rGhrelin (1-11), 25. Ghrelin (1-10), 26. Ghrelin (1-9), 27. Ghrelin (1-8), and 30. Ghrelin (1-4). Then, these fragments are isolated by analytical HPLC and measured for their activity. 41. [N-Acetyl]-Ghrelin (1-10) can be prepared in a usual manner by treating Ghrelin (1-10) with N-acetylsuccinimide. Human and rat ghrelin can also be made by use of a natural material.

Abbreviations

HMP resin; 4-hydroxymethyl-phenoxymethyl resin

Fmoc amide resin; 4-(2′,4′-dimethoxyphenyl-Fmoc-aminomethyl) phenoxyacetamido-ethyl resin

PAM resin; phenylacetoamidomethyl resin

HBTU; 2-(IH-benzotriazole-1-yl)-1,1,3,3-tetramethyluronium hexafluorophosphate

TBTU; 2-(1 H-benzotriazole-1-yl)-1, 1,3,3-tetramethyluronium tetrafluoroborate

HOBt; 1-hydroxybenzotriazole

DCC; dicyclohexylcarbodiimide

DI PCI; diisopropylcarbodiimide

TFA; trifluoroacetic acid

DIPEA; diisopropylethylamine

TIPS; triisopropylsilane

Fmoc; fluorenylmethoxycarbonyl

Boc; t-butyloxycarbonyl

Trt; trityl

Bu; t-butyl

Pmc; 2,2,5,7,8-pentamethylchroman-6-sulfonyl

Prl; propionyl

PhPrl; phenylpropionyl

Bzl; benzyl

Bom; benzyloxymethyl

Tos; toluenesulfonyl

Cl-Z; 2-chloro-benzyloxycarbonyl

Pis; 2-phenylisopropyl

Mtt; 4-methyltrityl

DMF; N,N-dimethylformamide

NMP; N-methylpyrrolidone

DMAP; 4-dimethylaminopyridine

HOSu; N-hydroxysucciniimide

Adod; 2-aminododecanoic acid

Aib; 2-aminoisobutylic acid

Ape; 5-aminopentanoic acid

Cha; cyclohexylalanine

Dap; 2, 3-diaminopropionic acid

Nal; naphtylalanine

Nle; norleucine

Protecting amino acids which can be used in synthesis

Fmoc method:

Boc-Gly, Fmoc-Gly, Fmoc-Ser (Bu), Fmoc-Ser (Trt), Fmoc-Glu (OBu), Fmoc-His (Boc), Fmoc-Gln (Trt), Fmoc-Arg (Pmc), Fmoc-Lys (Boc), Fmoc-Pro, Fmoc-Leu, Fmoc-Ala, Fmoc-Val, Fmoc-Phe, Fmoc-Phe, Fmoc-Ser (n-C8H17), Fmoc-Ser (n-C8H17), Fmoc-Cys (n-C8H17), Fmoc-Asp (OPis), Fmoc-Ser (Bzl), Fmoc-Cys (Trt), Fmoc-Dap (Octanoyl), Fmoc-2-Nal, Fmoc-2-Nal, Fmoc-Nle, Fmoc-Lys (Mtt), Fmoc-Aib-OH, Fmoc-Asp (O-C7H15) Boc method:

Boc-Gly, Boc-Ser (Bzl), Boc-Ser (Ac), Boc-Ser (Prl), Boc-Glu (OBzl), Boc-His (Bom), Boc-Gln, Boc-Arg (Tos), Boc-Lys (Cl-Z), Boc-Pro, Boc-Leu, Boc-Ala, Boc-Val, Boc-Phe, Boc-Cys (n-C8H17), Boc-Ape Boc-Ser (n-C8H17)

Units used

(a) Analytical HPLC system

Unit: Shimadzu LC-10A System

Column: YMC PROTEIN-RP (4.6 mm phi×150 mm)

Column temperature: 40 DEG C.

Eluent: A linear gradient of from 0 to 50% acetonitrile for 20 minutes in 0.1% trifluoroacetic acid

Flow rate: 1 mL/min.

Detection: UV (210 nm)

Injection volume: 10 to 100 mu I

Preparative HPLC system

Unit: Waters 600 Multisolvent Delivery System

Columns:

YMC-Pack-ODS-A (5 mu m, 20 mm×250 mm)

YMC-Pack-PROTEIN-RP (5 mu m, C4, 10 mm×250 mm)

YMC-Pack PROTEIN-RP (5 mu m, C4, 20 mm×250 mm)

YMC PROTEIN-RP (4.6 mm phi×150 mm)

Eluent: A suitable linear gradient of acetonitrile concentration in 0.1% trifluoroacetic acid

Flow rate: 10 mL/min. (for the column of an inner diameter of 20 mm), 3 mL/min. (for the column of an inner diameter of 10 mm), 1 mL/min. (for the column of an-inner diameter of 4.6 mm) Detection: 210 nm, 260 nm

Injection: 10 to 2000 mu 1 (2000 mu I or more was injected via a pump)

(c) Mass spectrometer

Unit: Finigan MAT TSQ700

Ion source: ESI

Detection ion mode: Positive

Spray voltage: 4.5 kV

Capillary temperature: 250 DEG C.

Mobile phase: A mixture of 0.2% acetic acid and methanol (1:1)

Flow rate: 0.2 mL/min.

Scan range: m/z 300 to 1,500

(d) Analysis of amino acid sequence

Unit: Applied Biosystem 477A, 492 model sequencer manufactured by Perkin Elmer

(e) Analysis of amino acid composition

Unit: L-8500 model amino acid analyzer manufactured by Hitachi, Co., Ltd.

Sample: Unless otherwise specified, the sample is hydrolyzed with 6 M HCl at 110 DEG C. for 24 hours in a sealed tube.

Example of Synthesis of a Derivative Having Acyl Serine (Fmoc Method, Carboxyl-Terminal Amide Derivatives

hGhrelin: GSS(CO-C7H15)FLSPEHQRVQQRKESKKPPAKLQPR

Fmoc-Arg(Pmc)-HMP-resin (403 mg, 0.25 mmol, ABI Co., Ltd) is treated with 20% piperazine for 20 minutes and subjected repeatedly to introduction of Fmoc-amino acid by HBTU/HOBt and elimination of Fmoc by piperazine sequentially to construct Fmoc-Ser(Bu)-Ser(Trt)-Phe-Leu-Ser(tBu)-Pro-Glu(OBu)-His(B oc)-Gln(Trt)-Arg(Pmc)-Val-Gln(Trt)-Gln(Trt)-Arg(Pmc)-Lys(Bo c)-Glu(OBu)-Ser(Bu) -Lys(Boc)-Lys(Boc)-Pro-Pro-Ala-Lys(Boc)-Leu-Gln(Trt)-Pro-Arg(Pmc)-resin. After Boc-Gly is finally introduced by DCC/HOBt, the resulting protected peptide resin (1.3 g) is treated with 1% TFA-5% TIPS-methylene chloride solution (15 mL) for 30 minutes. The peptide resin is filtrated, washed several times with methylene chloride (30 mL), and washed with 5% DIEA (10 mL) and then with methylene chloride (30 mL). The resulting de-Trt peptide resin (about 1.3 g) is swollen with NMP (10 mL), and octanoic acid (144.2 mg, 1.0 mmol) and DIPCl (126.2 mg, 1.0 mmol) are added thereto in the presence of DMAP (61.1 mg, 0.5 mmol) and allowed to react for 8 hours. The resin is recovered by filtration and washed with NMP and then with methylene chloride, followed by drying under vacuum to give about 1.2 g protected peptide resin wherein the side chain of 3rd serine is octanoylated. To this product is added a de-protecting reagent (10 mL) consisting of 88% TFA-5% phenol-2% TIPS-5% H2O, and the mixture is stirred at room temperature for 2 hours. The resin is removed by filtration, and the filtrate is concentrated followed by adding ether to the resulting residues to form precipitates. The precipitates are recovered by filtration and dried to give about 550 mg crude peptide. 200 mg of this product is dissolved in 10 mL water and applied to YMC-Pack PROTEIN-RP (C4, 20 mm×250 mm) and eluted with a linear gradient (flow rate: 10 mL/min.) for 60 minutes of from 0 to 54% acetonitrile in 0.1% trifluoroacetic acid. The desired fractions are collected and lyophilized to give about 120 mg of the desired product.

Example of Synthesis of a Derivative Having Acyl Serine (Fmoc Method, Carboxyl-Terminal Amide Compoundsi)

Ghrelin (1-9)-NH2; GSS(CO-C7H15)FLSPEH-NH2

Arg(Pmc)-Val-Gln(Trt)-Gln(Trt)-Arg(Pmc)-Lys(BFmoc-amide-resin (403 mg, 0.25 mmol, ABI Co., Ltd) is treated with 20% piperazine for 20 minutes and subjected repeatedly to introduction of Fmoc-amino acid by HBTU/HOBt and elimination of Fmoc by piperazine sequentially to construct Fmoc-Ser(Bu)-Ser(Trt)-Phe-Leu-Ser(Bu)-Pro-Glu(OBu)-His(Bo c)-resin. After Boc-Gly is finally introduced by DCC/HOBt, the resulting protected peptide resin (about 550 mg) is treated with 1% TFA-5% TIPS-methylene chloride solution (10 mL) for 30 minutes. The peptide resin is recovered by filtration, washed several times with methylene chloride (30 mL), and washed with 5% DIEA (10 mL) and then with methylene chloride (30 mL). The resulting de-Trt peptide resin (about 750 mg) was swollen with NMP (10 mL), and octanoic acid (144.2 mg, 1.0 mmol) and DIPCl (126.2 mg, 1 mmol) are added thereto in the presence of DMAP (61.1 mg, 0.5 mmol) and allowed to react for 4 hours. The resin is recovered by filtration and washed with NMP and then with methylene chloride, followed by drying under vacuum to give about 800 mg protected peptide resin wherein the side chain of 3rd serine is octanoylated. TFA (10 mL) is added to this product and stirred at room temperature for 30 minutes. The resin is removed by filtration, and the filtrate is then concentrated followed by adding ether to the resulting residues to form precipitates. The precipitates are recovered by filtration and dried to give about 250 mg crude peptide. About 200 mg of this product is dissolved in 10 mL of 30% aqueous acetic acid and applied to YMC-Pack PROTEIN-RP (C4, 20 mm×250 mm) and eluted with a linear gradient (flow rate: 10 mL/min.) for 60 minutes of from 0 to 54% acetonitrile in 0.1% trifluoroacetic acid. The desired fractions are collected and then lyophilized to give about 150 mg of the desired product.

Example of Synthesis of a Derivative Having Acyl Serine (Boc Method)

[Ser3(Propionyl)]-rGhrelin (1-28);
GSS(CO-CH2CH3)FLSPEHQKAQQRKESKKPPAKLQPR

Protected rat ghrelin resin (4-28) is constructed from Boc-Arg (Tos)-Pam resin (0.75 g, 0.5 mmol) by Boc chemistry, and Boc-Ser (CO—CH2CH3)-OH, Boc-Ser (Bzl)-OH and Boc-Gly-OH are condensed with a half (1.4 g) of the resin. The resulting resin, 1.5 g, is then treated with a mixture of HF and p-cresol (8.5 mL: 1.5 mL) at 0 DEG C. for 1 hour, and the HF is evaporated. Ether is added to the residues, whereby 671 mg crude peptide is obtained. This sample is then dissolved in 50% acetic acid (AcOH) and applied to a preparative column YMC-Pack-ODS-A (5 mu m, 20 mm×250 mm) and eluted at a rate of 10 mL/min. by a gradient of from 0 to 95% acetonitrile concentration in 0.1% TFA solution for 75 minutes. Those fractions containing the desired product are lyophilized to give approximately 135.8 mg crude peptide. A part (0.5 mg) of this product is applied to YMC-A-302 column (C18, 4.6 mm×l 50 mm) and eluted at a flow rate of 1 mL/min. by a gradient of from 15 to 19% concentration acetonitrile. This purification procedure is then repeated and the desired fractions are combined to give approximately 0.41 mg of the desired product. Ser(Bu)-Ser(Trt)-Phe-Leu-Ser(Bu)-Pro-Glu(OBu)-His(Bo c)-resin. Other compounds according to the invention can be produced likewise.

### Example 3

A randomised, single centre, four-period cross-over trial to investigate the absolute bioavailability of iv administered Ghrelin and sc administered Ghrelin at three different single doses in healthy subjects.

Objectives:

Primary: To investigate the absolute bioavailability of three different doses of Ghrelin administered as single iv and sc doses.

Secondary: 1) To investigate the dose linearity (dose proportionality) of the ascending doses. 2) To investigate and compare the pharmacodynamic profiles between the treatments. 3) To assess the safety and local tolerability.

Trial Design:

A randomised, single centre, unbalanced block design, four-period cross-over trial to investigate the absolute bioavailability between iv administered Ghrelin and sc administered Ghrelin at three different single doses in healthy subjects. Three doses will be used for each way of administration: low, medium and high. To reduce the number of dosings to each individual and hence reduce the length of the trial each subject will only receive four doses of the total of six doses, ie. two dose levels administered as iv and sc, respectively. The unbalanced block design will ensure that all three-dose levels will be covered in this way although not all subjects will receive all dose levels. A sufficient washout period will be placed between the individual dosing periods.

Endpoints:

Pharmacokinetics of Ghrelin:

AUC₀₋ₜ, AUC, Cₘₐₓ, tₘₐₓ, t_{1/2}, Cl/f, Vz/f, Cl, Vz, , t½, MRT

Pharmacodynamics:

GH: AUC, Cmax and tmax

Cardiac output, assessment of hunger, food/energy intake, degree of pleasure related to food intake, body mass, energy expenditure, DEXA.

Safety:

Safety and local tolerability will be assessed throughout the study by clinical evaluations (physical examination and vital signs), electrocardiography and laboratory tests (hematology and clinical chemistry).

Trial population and power calculation:

Healthy male subjects, aged 18-45 years with a body mass index (BMI) of 19-26 kg/m² (both inclusive).

The primary objective of this study is to investigate the absolute bioavailability of ghrelin administered as iv and sc. An unbalanced block design will be applied to reduce the trial period time and reduce the number of dosings per subject. The number of subjects needed to perform a statistical analysis of absolute bioavailability per dose levels as well as an analysis of dose linearity between doses will be calculated based on existing literature data.

Trial Products:

Ghelin for iv and sc administration.

### Example 4

One patient with established cancer cachexia and reduced appetite, who responded well to weekly IV injections of ghrelin, has been treated with subcutaneous ghrelin. The patient suffers from mesothelioma, which did not appear to respond infavorably to the iv ghrelin treatment.

Treatment

The patient was treated with subcutaneous injections of 0.8 to 1.5 micrograms/kg ghrelin twice daily for 4 weeks. The injections were taken before meals.

Assessments

Pharmacokinetic and pharmacodynamic assessment was performed at day 1 and 2 by blood sampling before and after each injection and measurement of plasma levels of growth hormone and active and total ghrelin.

The patient was followed weekly by standard laboratory tests, Cachexia/anorexia and quality of life questionnaires, body-weight, body-composition analysis (DEXA-scan) and chest-x-ray or thorax-CT among others.

Results

The patient had stable body weight and body composition. He did not feel more intensified hunger, but was able to eat full plates of meals and finish these, which he had not been able to prior to the treatment.

The tumor did not progress during the subcutaneous ghrelin treatment as documented by CT-scans. Lab values did not show a substantial difference going beyond the variability inherent to such a sick patient.

Measurements of plasma growth hormone before and after the two subcutaneous injections shows a clear increase in growth hormone levels of more than 5 fold (see FIG. 11), thus documenting that the injected ghrelin is active and exerts its effects in the patient.

Conclusions:

Daily subcutaneous injections of ghrelin show significant advantages in the treatment of patients with cancer cachexia and reduced appetite. The treatment resulted in stable body-weight over the 4 week period and increased objective hunger and ability to eat meals. Furthermore, the treatment had no significant adverse effects, and the tumor did not progress during the treatment.

### Example 5

Functional Tests on the Ghrelin Receptor

Transfections and tissue culture—COS-7 cells were grown in Dulbecco's modified Eagle's medium 1885 supplemented with 10% fetal calf serum, 2 mM glutamine and 0.01 mg/ml gentamicin. Cells were transfected using calcium phosphate precipitation method with chloroquine addition as previously described (Holst et al. Mol. Pharm (1998); 53;1; p 166-175, “Steric hindrance mutagenesis versus alanine scan in mapping of ligand binding sites in the tachykinin NK1 receptor”). For gene dose experiments variable amounts of DNA were used. The amount of cDNA(20 μg/75 cm²) resulting in maximal signaling was the used for dose responds curves. HEK-293 cells were grown in D-MEM, Dulbecco's modified Eagle's medium 31966 with high glucose supplemented with 10% fetal calf serum, 2 mM glutamine and 0.01 mg/ml gentamicin. Cells were transfected with Lipofectamine 2000 (Life Technologies).

Phosphatidylinositol turnover—One day after transfection COS-7 cells were incubated for 24 hours with 5 μCi of [³H]-myo-inositol (Amersham, PT6-271) in 1 ml medium supplemented with 10% fetal calf serum, 2 mM glutamine and 0.01 mg/ml gentamicin per well. Cells were washed twice in buffer, 20 mM HEPES, pH 7.4, supplemented with 140 mM NaCl, 5 mM KCl, 1 mM MgSO₄, 1 mM CaCl₂, 10 mM glucose, 0.05% (w/v) bovine serum; and were incubated in 0.5 ml buffer supplemented with 10 mM LiCl at 37 C for 30 min. After stimulation with various concentrations of peptide for 45 min at 37 C, cells were extracted with 10% ice-cold perchloric acid followed by incubation on ice for 30 min. The resulting supernatants were neutralized with KOH in HEPES buffer, and the generated [³H]-inositol phosphate was purified on Bio-Rad AG 1-X8 anion-exchange resin as described. Determinations were made in duplicates.

CRE, SRE and NF-κ-B reporter assay. HEK293 cells (30 000 cells/well) seeded in 96-well plates were transiently transfected. In case of the CRE reporter assay the cells were transfected with a mixture of pFA2-CREB and pFR-Luc reporter plasmid (PathDetect CREB trans-Reporting System, Stratagene) or SRE-Luc (PathDetect SRE Cis-Reporting System, Stratagene) and the indicated amounts of receptor DNA. Following transfection cells were maintained in low serum (2.5%) throughout the experiments and were treated with the respective inhibitor of intracellular signaling pathways. One day after transfection, cells were treated with the respective ligands in an assay volume of 100 μl medium for 5 hrs. The assay was terminated by washing the cells twice with PBS and addition of 100 μl luciferase assay reagent (LucLite, Packard). Luminescence was measured in a TopCounter (Top Count NXT™, Packard) for 5 sec. Luminescence values are given as relative light units (RLU).

MAP Kinase assay: COS 7 cells (seeding density 150.000 cells/well) were transfected in the assay plates. Two days after transfection the indicated concentration of ligand were added to assay medium without any serum and incubated for 10 min at 37 C. The reaction were stopped by removing the medium and two washing steps with ice cold PBS. The cells were lysed in sample buffer and separated on SDS/10% PAGE according to Laemmi (“Cleavage of structural proteins during the assembly of the head of bacteriophage T4” Nature vol 227, p 680-685). Proteins were transferred onto nitrocellulose and Western blot analysis carried out using 1:5000 dilution of mouse monoclonal antiphopho-ERK1/2 anti-body (Santa Cruz Biotechnology). Total ERK protein was determined using a 1:10000 dilution of anti-ERK antibody (Santa Cruz Biotechnology). Blots were probed anti mouse horseradish peroxidase-conjugated secondary antibodies, visualised using enhanced chemiluminiscence reagent (Amersham Bioscience, New Jersey, US) and quantified by densiometric analysis. ERK1/2 phosphorylation was normalized according to the loading of protein by expressing the data as a ratio of phopho-ERK1/2 over total ERK1/2. Results were expressed as percentage of the value obtained in non stimulated mock transfected cells.

### Example 6

Efficacy of Subcutaneous Administration of Ghrelin

6+6 mice received 12 or 24 nmol of ghrelin by a subcutaneous bolus injection in the neck (corresponding to 400 and 800 nmol kg⁻¹, respectively). Blood was sampled (orbital puncture (Salehi et al., J Physiolo 1999; 514:579-591)) before injection (time 0) and 15, 60,120, 240 and 300 min after injection. Immunoactive ghrelin was measured in 20 μl plasma with a radioimmunoassay (RIA) that uses an antiserum raised against acylated human ghrelin (Phoenix Pharmaceuticals); ¹²⁵I-labelled ghrelin-28 was used as a tracer a rat ghrelin-28 as standard. This antiserum recognises both octanoylated and des-octanoylated ghrelin-28 but does not recognise des-Gln¹⁴ ghrelin. Plasma concentrations were expressed as pmol equivalents of rat ghrelin-28 per liter. Active (octanoylated) ghrelin was determined in 50 μl plasma using an enzyme linked immunosorbent assay (ELISA) kit (LINCO Research, St. Charles, Mo., USA). This antiserum does not recognise des-octanoylated ghrelin.

Results:

(Means are presented ±S.E.M)

Subcutaneous injections (arrows) of 12 and 24 nmol of ghrelin to mice (n=6 in each group) raised the plasma ghrelin concentration (see FIG. 1A); plasma ghrelin was still elevated 6 h after injection of 12 nmol of ghrelin (see FIG. 1B). Plasma concentration of active (octanoylated) ghrelin in mice (n=4) was greatly elevated in response to 12 nmol of ghrelin (see FIG. 1C); again, these levels remained high 6 h after injection (see FIG. 1D).

### Example 7

Efficacy of Subcutaneous Administration of Ghrelin to Treat Gastrectomised Individuals

Mice were subjected to gastrectomy or to sham operation. Gastrectomy was carried out by resecting the glandular stomach (antrum and fundus), followed by anastomozing the duodenum and the rumen end-to-end. The mortality associated with gastrectomy was 10%. Some of the gastrectomised and sham-operated mice received a subcutaneous dose of ghrelin (12 nmol) daily for 8 weeks. Controls received saline. On the first 14 days the injections were given in the morning (8-9 am), subsequent injections were made in the afternoon (17-18 pm; the consequent response in terms of weight gain did not differ, hence the timing of the injections may be varied). After 8 weeks the mice were killed by decapitation (16-18 h after the last injection). Blood was collected and plasma stored at −20° C. for measurement of ghrelin (see below). Lean tissue was analysed by dual energy x-ray absorptiometry (DXA) (PIXImus, Lunar Corporation, Madison, Mich., USA). White adipose tissue (WAT) (mesenteric, retroperitoneal-renal, gonadal and inguinal) and intrascapular brown adipose tissue (BAT) was dissected out and weighed

Measurement of circulating ghrelin: If not otherwise stated, determination of circulating ghrelin was carried out on plasma from animals at sacrifice (end-point analysis). Immunoactive ghrelin was measured in 20 pi plasma with a radioimmunoassay (RIA) that uses an antiserum raised against acylated human ghrelin (Phoenix Pharmaceuticals); ¹²⁵I-labelled ghrelin-28 was used as a tracer a rat ghrelin-28 as standard. This antiserum recognises both octanoylated and des-octanoylated ghrelin-28 but does not recognise des-Gln¹⁴ ghrelin. Plasma concentrations were expressed as pmol equivalents of rat ghrelin-28 per liter. Active (octanoylated) ghrelin was determined in 50 μl plasma using an enzyme linked immunosorbent assay (ELISA) kit (LINCO Research, St. Charles, Mo., USA). This antiserum does not recognise desoctanoylated ghrelin.

Statistical analysis: Values are expressed as means±S.E.M. Differences were analyzed by the student t-test or by one-way analysis of variance (ANOVA), using Bonferroni's multiple comparison test. A p value of less than 0.05 was considered statistically significant.

Results:

i) Ghrelin levels:

Plasma concentration of ghrelin were measured in mice 8 weeks after sham operation or gastrectomy with or without the daily subcutaneous administration of ghrelin (12 nmol sc)—see FIG. 2. Gastrectomy reduced the plasma ghrelin concentration. The plasma ghrelin concentration was elevated 16-18 h after injection of ghrelin.

ii) Weight gain:

The body weight of mice was lowered (−15%) by gastrectomy (see FIG. 3). Daily subcutaneous injections of ghrelin (12 nmol sc) raised the body weight (8%) in gastrectomised mice but not in sham-operated mice.

iii) Body composition:

Fat depots: gastrectomy reduced the amount of fat in mice (−30%). Daily administration of ghrelin (12 nmol sc) normalized the amount of fat in the gastrectomised mice and raised it in the sham-operated animals (21%) (see FIG. 4).

Lean body mass: Gastrectomy reduced the amount of lean tissue in mice (−11%). Daily subcutaneous administration of ghrelin (12 nmol sc) normalized the amount of lean tissue in the gastrectomised mice (see FIG. 5).

### Example 8

Examples of Questionnaires Assessing Patient Quality of Life

We are interested in information regarding you and your health. Please answer the follow
questions by ticking off the number that applies best to you. There are no “right” or
“wrong” answers. This information will be treated with confidentiality.

| | | not at all | less | moderately | very |
|---|---|---|---|---|---|
| A) EORTC QLQ-C30 | | | | | |
| 1. | Is physical exertion (i.e. carrying a heavy | 1 | 2 | 3 | 4 |
| | shopping bag or a suitcase) causing you a problem? | | | | |
| 2. | Is a longer walk causing you a problem? | 1 | 2 | 3 | 4 |
| 3. | Is a short walk outside your home causing you | 1 | 2 | 3 | 4 |
| | a problem? | | | | |
| 4. | Are you spending major part of the day in bed | 1 | 2 | 3 | 4 |
| | or in a chair? | | | | |
| 5. | Do you require help with eating, getting dressed, | 1 | 2 | 3 | 4 |
| | having a wash or using the toilet | | | | |
| During the last half week: | | | | | |
| 6. | were you restricted in your work or in other | 1 | 2 | 3 | 4 |
| | daily activities? | | | | |
| 7. | were your restricted in doing your hobbies or | 1 | 2 | 3 | 4 |
| | other leisure pursuits? | | | | |
| 8. | were you short of breath? | 1 | 2 | 3 | 4 |
| 9. | were you in pain? | 1 | 2 | 3 | 4 |
| 10. | did you have to rest? | 1 | 2 | 3 | 4 |
| During the last half week: | | | | | |
| 11. | did you have trouble sleeping? | 1 | 2 | 3 | 4 |
| 12. | did you feel faint? | 1 | 2 | 3 | 4 |
| 13. | did you have no appetite? | 1 | 2 | 3 | 4 |
| 14. | did you feel ill? | 1 | 2 | 3 | 4 |
| 15. | did you vomit? | 1 | 2 | 3 | 4 |
| 16. | were you constipated? | 1 | 2 | 3 | 4 |
| 17. | did you have diarrhea? | 1 | 2 | 3 | 4 |
| 18. | were you tired? | 1 | 2 | 3 | 4 |
| 19. | were you disturbed in your daily life | 1 | 2 | 3 | 4 |
| | because of pain? | | | | |
| 20. | did you have a problem concentrating on things | 1 | 2 | 3 | 4 |
| | (i.e. reading the newspaper or watching the | | | | |
| | television)? | | | | |
| 21. | were you tense? | 1 | 2 | 3 | 4 |
| 22. | did you worry? | 1 | 2 | 3 | 4 |
| 23. | were you irritable? | 1 | 2 | 3 | 4 |
| 24. | did you feel depressed? | 1 | 2 | 3 | 4 |
| 25. | did you have problems remembering things? | 1 | 2 | 3 | 4 |
| 26. | was your family life disturbed by your physical | 1 | 2 | 3 | 4 |
| | condition or your medical treatment? | | | | |
| 27. | was your social life or your undertakings | 1 | 2 | 3 | 4 |
| | with other people disturbed by your physical | | | | |
| | condition or your medical treatment? | | | | |
| 28. | has your physical condition or your medical | 1 | 2 | 3 | 4 |
| | treatment caused you financial problems? | | | | |

With regard to the following questions please tick off the number from 1 to 7
that applies best to you.

| | |
|---|---|
| 29. | How would you estimate your health during the last half week? |

| | | | | | | |
|---|---|---|---|---|---|---|
| 1 | 2 | 3 | 4 | 5 | 6 | 7 |

| | |
|---|---|
| very bad | excellent |

| | |
|---|---|
| 30. | How would you estimate your quality of life during the last half week? |

| | | | | | | |
|---|---|---|---|---|---|---|
| 1 | 2 | 3 | 4 | 5 | 6 | 7 |

| | |
|---|---|
| very bad | excellent |

Further questions
Patients sometimes report the following complaints. Please indicate the extent
to which these complaints have occurred with you within the last half week.

| 31. | did you enjoy your meals | 1 | 2 | 3 | 4 |
|---|---|---|---|---|---|
| 32. | did you feel sufficiently nourished? | 1 | 2 | 3 | 4 |
| 33. | did you worry about your weight? | 1 | 2 | 3 | 4 |
| 34. | were you able to swallow normally and | 1 | 2 | 3 | 4 |
| | without any problems? | | | | |
| 35. | did most of the food taste unpleasant? | 1 | 2 | 3 | 4 |
| 36. | was your mouth dry? | 1 | 2 | 3 | 4 |
| 37. | did you loose appetite once you started | 1 | 2 | 3 | 4 |
| | to eat? | | | | |
| 38. | did you quickly feel full? | 1 | 2 | 3 | 4 |
| 39. | did family or friends urge you to eat more? | 1 | 2 | 3 | 4 |
| 40. | did you have a tense stomach? | 1 | 2 | 3 | 4 |
| 41. | were your worried that you might look | 1 | 2 | 3 | 4 |
| | too skinny? | | | | |
| 42. | have you avoided contact with other people | 1 | 2 | 3 | 4 |
| | because of your looks? | | | | |

B) Taste questionnaire
Name: Date: #:

| | |
|---|---|
| 1. | How disturbed is your taste sensation at the moment |

| | |
|---|---|
| Not disturbed | Very strongly disturbed |

| | |
|---|---|
| 2. | Please tick off the most appropriate answer: |

When you compare your taste sensation from before your illness with now:

| a) | Salt tastes: | 1. stronger |
|---|---|---|
| | | 2. the same |
| | | 3. weaker |
| | | 4. I do not taste it at all |
| b) | Sweet (like sugar) tastes: | 1. stronger |
| | | 2. the same |
| | | 3. weaker |
| | | 4. I do not taste it at all |
| c) | Sour (like lemon or vinegar): | 1. stronger |
| | | 2. the same |
| | | 3. weaker |
| | | 4. I do not taste it at all |
| d) | Bitter (black coffee, tonic water): | 1. stronger |

2. the same
3. weaker
4. I do not taste it at all

| | |
|---|---|
| 3. | Please tick off the appropriate answer: |

I have a constant bad taste in my mouth.

| | 1. never |
|---|---|
| | 2. rarely |
| | 3. sometimes |
| | 4. often |
| | 5. always |
| | 6. only during chemotherapy and on days immediately afterwards |
| 4. | How disturbing do you consider the taste changes? |
| 26. | extremely disturbing |
| 27. | very disturbing |
| 28. | moderately disturbing |
| 29. | slightly disturbing |
| 30. | not disturbing at all |
| 5. | Are you eating more or less because of the taste changes? |

More Less the same

| | |
|---|---|
| 6. | At the moment my taste sensation is: |

| | |
|---|---|
| Very good | Very bad |

C) Hunger-Appetite-Feeling of Supersaturation-
Nausea-Anxiety-Tiredness-Visual analogue scale

| Name: Date: #: Time: | [] before |
|---|---|
| | breakfast |
| | [] 10.15 |
| | [] 11.30 |
| | before lunch |
| | [] 12.30 |
| | after lunch |

| | |
|---|---|
| 1. | How do you estimate your appetite NOW? |

| | |
|---|---|
| Very good appetite | No appetite at all |

| | |
|---|---|
| 2. | How do you estimate your hunger NOW? |

| | |
|---|---|
| No hunger | Very strong hunger |

| | |
|---|---|
| 3. | How do you estimate your supersaturation feeling NOW? |

| | |
|---|---|
| No supersaturation feeling | Very strong supersaturation feeling |

| | |
|---|---|
| 4. | How do you estimate your nausea NOW? |

| | |
|---|---|
| No nausea | Very strong nausea |

| | |
|---|---|
| 5. | How do you estimate your anxiety NOW? |

| | |
|---|---|
| Great anxiety | No anxiety at all |

| | |
|---|---|
| 6. | How do you estimate your tiredness (fatigue, faintness) NOW? |

| | |
|---|---|
| No tiredness | Very strong tiredness |

### Example 9

Examples of Suitable Formulations for Preparing Pharmaceutical Compositions for Use in the Present Invention

Two different types of liposomes were made containing dipalmitoyl DL-α-phosphatidylcholine (DPPC ) and a mixture of phosphatidylcholin and cholesterol (PC/Chol), respectively. The liposomes were prepared by dissolving and mixing the lipids in chloroform. Chloroform was removed overnight by rotation evaporation, and the resulting lipid film was first striped with ethanol (99,9%) and then left overnight in the rotation evaporator. The multilamellar liposomes were formed by hydration in HEPES-buffer (10 mM HEPES, 50 mM KCl, 1mM NaN₃, pH=5,5) for at least 1 h. The hydration temperature was 51° C. (10° C. above the Tₘ of the phospholipids). Subsequently, the liposomes were sonicated for 30 sec every 10 min during one hour using a tip sonicator. One hundred nanometer unilamellar liposomes were made from the multilamellar liposomes by extrusion through 100 nm polycarbonate filters. Size measurements were performed by dynamic light scattering (DLS) using a Zetasizer 4 (Malvern, UK). Tₘ were determined by Differential Scanning Caleometry (DSC; MicroCal™ Incorporated). Ghrelin were added approximately 2 h before the administration of the formulation to the animals in doses of 60 μg per 500 μL

Characterization:

Size of the DPPC liposomes (Z average): 106,3 nm

Polydispersity of the DPPC liposomes: 0,29

Concentration of DPPC: 2 mM

Size of the PC/Chol liposomes (Z average): 108,4 nm Polydispersity of the PC/Chol liposomes: 0,20

Concentration of cholesterol: 0.6 mM

Concentration of phosphatidylcholine: 1.4 mM

Intralipid 30% were purchased from the local Pharmacy on the Danish University Hospital Copenhagen (Rigshospitalet). 1000 mL contain: purified soybean oil 300 g, purified egg phospholipids 12 g, glycerol anhydrous 16.7 g, water for injection q.s. ad 1 000 mL. pH is adjusted with sodium hyroxide to pH approximately 7.5.

### Example 10

Animal Study to Investigate Pharmacokinetics of Ghrelin Formulations Described in Example 9. See FIG. 10 for Overview of Study

Animals: Thirty (30) male Sprague Dawley rats (aged 6-7 weeks; approximately 300 g; Charles River, Germany) were used. Upon arrival at the animal facility, they were housed 3 per cage for one week, and subsequently transferred to individual cages with access to food and water ad libitum.

From the arrival date, rats were kept under a 12/12 L/D cycle lights on at 0600 and in temperature and humidity controlled rooms.

Treatment Groups and Randomization

After one week of singly housing, the rats were ready for experiments and were randomized into 5 weight-matched groups (6 pr. group):

| | |
|---|---|
| Group 1 | Vehicle |
| Group 2 | Ghrelin in buffer (200 mg/kg) |
| Group 3 | Ghrelin in intralipid (200 mg/kg) |
| Group 4 | Ghrelin in Chol/PC (200 mg/kg) |
| Group 5 | Ghrelin in DPPC (200 mg/kg) |

All compounds were administered by subcutaneous administration (0.5 ml/300g rat)

Experimental Procedure

Three days prior to experiment start the rats received a mock sc injection daily with NaCl (0.9%) to accustom them to the experimental procedure and to reduce injection related stress.

On the experimental day (0800-0930), 15 minute prior to receiving the test dose, a 200 μl blood sample was drawn from the tail vein. At time point 0 a second blood sample was drawn and immediately after rats received an sc injection with test compound according to the groups listed above. Seven more samples were drawn (volume 200 μl) at time points: 15, 30, 45, 60, 90, 120 and 240 minutes post injection according to study outline. The total blood volume drawn was 1800 μl (approx. 10% of total blood volume).

Blood samples were kept on ice until plasma separation could be performed. After centrifugation (4000 rpm for 10 minutes) plasma was transferred to vials/tubes containing 5 μL 1N HCl og 5 μL 2mg/mL solution of Phenylmethylsulfonyl fluoride in isopropanol.

The active (acylated) ghrelin in the plasma samples was measured by use of a RIA assay (Linco Research, Inc) according to the manufactures protocol.

Results:

In the pharmacokinetic study there was an approximately 10 fold increase in the active ghrelin after subcutaneous administration of ghrelin (200 mg/kg) in a buffer. The maximal plasma concentration of ghrelin was observed after 15 min and decreased approximately 30% the following 15 min (time point 30 min) and further 60% the following 15 min (time point 45). The plasma level of active ghrelin was slightly increased after 120 min, however back to basal level after 240 min.

Administration of ghrelin formulated in lipid emulsion did not change the maximal achieved level of ghrelin, Administration of ghrelin formulated in lipid emulsion did not change the maximal achieved level of ghrelin, however there was a tendency to an increased terminal half-life of active ghrelin (FIG. 9A), indicative of a depot effect. Ghrelin was also administrated in two different liposome formulations containing dipalmitoyl DL-α-phosphatidylcholine (DPPC ) or a mixiture of phosphatidylcholin and cholesterol (PC/Chol), respectively. The PC/Chol liposomes did not increase the maximal achieved plasma level of active ghrelin but the plasma concentration after 45 min were higher after administration of PC/Chol liposome formulated ghrelin compared to ghrelin in buffer (FIG. 9B). Administration of ghrelin formulated in DPPC liposomes increased the maximal achieved level of ghrelin almost two fold at time point 15 min. Interestingly, the plasma level of ghrelin was increased more than two fold after 30 min and 45 min (FIG. 9C).

Conclusions:

Subcutaneous injections of ghrelin resulted in significant increases in circulating plasma levels, irrespective of formulation. Formulation in lipid appeared to result in a slight depot effect, since the ghrelin concentration decreased at a slower rate than when formulated in saline. Formulation in DPPC liposomes resulted in a two fold increase in active ghrelin levels without any clear effect on the half-life.

### Example 11

Administering Ghrelin to Cancer Cachexia Patients (see FIGS. 6 and 7)

Ghrelin is administered to patients with cancer cachexia in order to improve their food intake and nutritional status over an 8 week period. Compound used in trial is produced by an authorized GMP-production facility. Wt human ghrelin (GMP grade) with acetate as the counter ion was purchased from Polypeptide Laboratories, Inc. (CAS no: SARA313). The purity of the peptide as assessed by HPLC was between 98.41 and 99.63%, respectively. The peptide content of the substance to be used in this example is 84,3%, the remainder being acetate as counter ion. The substance is dissolved in physiological saline (0.9%) on the day of injection.

A in one trial set up patients are treated with a single infusion of either ghrelin (dose range from 100 pmol/kg to 1 mol/kg) or saline. The patients have been fasted 16h before the administration. During the infusion, the patient will be monitored every 30 min according to the table shown in FIG. 9. After 30 min infusion the patient will be offered a buffet for food selection and intake. This serves as a control to ensure sufficient activity of the active compound. The acute effects of ghrelin are evaluated by the amount of food ingested from the free buffet, compared to placebo treatment. In a second round of experiments lasting for 8 weeks patients are treated with either a high dose of ghrelin ranging from 1 μg/kg-80 μg/kg s.c. ghrelin or a low dose of ghrelin ranging from 0.1 μg/kg-1.0 pg/kg s.c. ghrelin. The treatment is given by the Swedish home care system as daily subcutaneous injection on the abdomen, approximately at 10 am in the morning as injections on the abdomen. After 4 weeks of treatment, the patients will be objectively evaluated by controlled diary for food intake and by blood sampling. Furthermore, the patients are evaluated at the start and end, respectively of the trial period by controlled diary for food intake, blood sampling, exercise level, resting energy expenditure, body composition, full physical examination, standard blood-biochemistry as described, and health related quality of life among others. Assessments at the respective visits will occur as shown in FIG. 9.

Patients:

Patients with documented cancer Cachexia and—Documented cancer Cachexia with significant weight-loss in the preceeding period and reduced appetite.

The Cachexia may be caused by any type of cancer, including oesophageal, lung, breast, gastric, pancreatic, neurological and urinary tract, bone, haematological, reproductive tract, exocrine gland, endocrine gland, multiple endocrinological neoplasms, testicular, prostate, nephrological, skin, thyroid, liver, and colon

Efficacy of ghrelin action will be assessed according to FIG. 9:

Clinical Assessments:

Acute Food intake: Dietician assessed food intake during the infusion and the subsequent 2½ h.

Chronic Food intake: A daily report of the amount of food consumed during the day, and assessment of the pleasantness related to the food intake. This will be validated by urine nitrogen excretion, based on 4 day diet diary.

Body-weight: Standard and calibrated scale will be used at the clinic.

Resting energy expenditure (REE) is a very important measurement, since it is affected both by the state of the disease and the body size.

Exercise test

Actigraph—is used according to standard protocol described on the homepage (www.mtiactigraph.com)

Health related QoL using standard forms as described elsewhere

Para-clinical Assessments:

Nitrogen excretion in the urine: 24 h urine collection should be used as validation of the reported food intake.

Plasma glucose, plasma FFA, plasma triglycerides, plasma glycerol and plasma amino acids: Plasma substrates measured to ensure the reported food intake is in accordance with the absorbed amount of food intake.

Lean body mass and-fat mass assessed by TSF thickness and mid-arm circumference as a measurement of body composition.

Total body fat (and fat free mass) will be assessed DEXA scan, using software 1.31 for the lunar DPX-L (Scanexport Medical, Helsingborg, Sweden).

Plasma Leptin: Leptin is produced by and secreted from the fat cell. The plasma level of leptin gives an estimate of the total fat cell burden.

Plasma Ghrelin: The basal ghrelin level tends to be increased in cachectic patients.

Plasma-GH: In previous studies GH has been measured as a control for the effect of ghrelin administration (Enomoto et al., 2003).

IGF-I: A single determination of IGF-I summarizes 24 h of GH secretion. This has been demonstrated in healthy volunteers where levels of circulating IGF-I have been shown to correlate with spontaneous GH secretion (Rose N Engl J Med 1988;319:201-207). IGF-I may also increase independently of GH increase by improved nutritional status.

IGFBP-3: One of the carrier proteins for IGF-1. It increases in parallel with IGF-I but with a slower response rate.

Albumin: Is an indicator of nutritional status

Prealbumin: Indicator of nutritional status, with quicker response to alterations than albumin.

Cortisol: Ghrelin administration has been shown to increase the serum cortisol level (Broglio et al., 2003a). Corticosteroids have been shown to have a significant anti nausea effect and to improve asthenia and pain control, which may be beneficial for the cachectic cancer patient. However, cortisol has never been shown to increase weight in cachectic cancer patients.

CRP and ESR: Acute phase proteins and ESR are often good indicators of systemic inflammation related to the cancer process (Inui, 2002)

### Example 12

Treatment of Patients with Cancer-Related Anorexia/Cachexia

Patients with advanced cancer suffering from the anorexia/cachexia syndrome (ACS), such as patients with any type of advances, incurable cancer, are believed to benefit from the present invention in terms of improved quality of life, increased appetite, increased food intake, maintenance or gain of weight, food pleasantness, and/or fat deposition.

Patients are treated for 60 minutes with ghrelin dissolved in 250 ml normal saline (NaCl 0.9%) at a dose of 10 pmol/kg/minute (equals 0.0336 mcg/kg/min).

Investigational treatment: ghrelin is available in GMP-quality in prepared vials of 88 mcg from Calbiochem-Novabiochem AG, CLINALFA, Merck Biosciences, Switzerland. Placebo consists of an empty 250 ml normal saline infusion, which will be provided by a hospital pharmacy. Ghrelin is dissolved in saline and dose of 0.0336 mcg/kg/min ghrelin will be administered to the patient.

Assessments of Efficacy:

eating related symptoms: assessed using an adapted version of the “Functional Assessment of Appetite and Cachexia therapy (FAACT) questionnaire; the EORTC-QLQ-30 Anorexia/Cachexia questionnaire; the NCCTG—Anorexia/Cachexia questionnaire and the Edmonton Sympton assessment scale.

Quality of life: will be assessed using the EORTC-QLQ-C30 questionnaire (see example 8)

Nutritional intake and food preferences: food intake measurement will be by percentage calculation of food products consumed at each meal by the patient, the clinical dietician will assess the food preferences as part of their routine assessments.

Food pleasantness: will be assessed after lunch using visual analogue scales, following established anchors

Perceived appetite, hunger, nausea and satiety: will be assessed in the morning, before infusion, and before and after lunch using visual analogue scale, following established anchors. We will also apply a shortened ad hoc taste questionnaire.

Growth hormone (GH): since GH reflects directly the biological function of ghrelin, with a rapid increase of GH after ghrelin injections, we will also monitor GH levels at the same time points as ghrelin. A standard ghrelin assay will be used.

Body composition: body compositions will be assessed by BMI, bioimpedance analysis and dual photon absorptiometry/dual energy x-ray absorptiometry (DEXA). Albumin and transferrin levels will be determined as parameters for nutritional status.

Cardiovascular autonomic function: for the screening of autonomic disorders a 20 minute holter ekg will be performed, and the SDNN value determined.

Mediators of the primary anorexia/cachexia syndrome: mediators of the proinflammatory reaction (CRP, IL-6, TNF-alpha), the activated metabolism (free fatty acids, triglycerides, insulin, glucose, leptin), the gut-brain axis (ghrelin), and the somatotrophic axis (IGF-1, free testosterone) will be determined as baseline in the first week. A urine sample will be asserved for assessment of proteolysis-inducing factor (PIF), a mediator of the paraneoplastic anorexia/cachexia syndrome.

### REFERENCE LIST

1. Kojima M, Hosoda H, Matsuo H, Kangawa K 2001 Ghrelin: discovery of the natural endogenous ligand for the growth hormone secretagogue receptor. Trends Endocrinol Metab 12:118-122
2. Nakazato M, Murakami N, Date Y, Kojima M, Matsuo H, Kangawa K, Matsukura S 2001 A role for ghrelin in the central regulation of feeding. Nature 409:194-198
3. Bowers C Y, Momany F, Reynolds G A, Chang D, Hong A, Chang K 1980 Structure-activity relationships of a synthetic pentapeptide that specifically releases growth hormone in vitro. Endocrinology 106:663-667
4. Bowers C Y, Momany F A, Reynolds G A, Hong A 1984 On the in vitro and in vivo activity of a new synthetic hexapeptide that acts on the pituitary to specifically release growth hormone. Endocrinology 114:1537-1545
5. Patchett A A, Nargund R P, Tata J R, Chen M H, Barakat K J, Johnston D B, Cheng K, Chan W W, Butler B, Hickey G,. 1995 Design and biological activities of L-163,191 (MK-0677): a potent, orally active growth hormone secretagogue. Proc Natl Acad Sci U S A 92:7001-7005
6. Smith R G, Cheng K, Schoen W R, Pong S S, Hickey G, Jacks T, Butler B, Chan W W, Chaung L Y, Judith F,. 1993 A nonpeptidyl growth hormone secretagogue. Science 260:1640-1643
7. Howard A D, Feighner S D, Cully D F, Arena J P, Liberator P A, Rosenblum C I, Hamelin M, Hreniuk D L, Palyha O C, Anderson J, Paress P S, Diaz C, Chou M, Liu K K, McKee K K, Pong S S, Chaung L Y, Elbrecht A, Dashkevicz M, Heavens R, Rigby M, Sirinathsinghji D J, Dean D C, Melillo D G, Van Der Ploeg L H,. 1996 A receptor in pituitary and hypothalamus that functions in growth hormone release. Science 273:974-977
8. Smith R G, Van Der Ploeg L H, Howard A D, Feighner S D, Cheng K, Hickey G J, Wyvratt M J, Jr., Fisher M H, Nargund R P, Patchett AA 1997 Peptidomimetic regulation of growth hormone secretion. Endocr Rev 18:621-645
9. Kojima M, Hosoda H, Date Y, Nakazato M, Matsuo H, Kangawa K 1999 Ghrelin is a growth-hormone-releasing acylated peptide from stomach. Nature 402:656-660
10. Banks W A, Tschop M, Robinson S M, Heiman ML 2002 Extent and direction of ghrelin transport across the blood-brain barrier is determined by its unique primary structure. J Pharmacol Exp Ther 302:822-827
11. Barber, M. D., J. A. Ross, and K. C. Fearon. “Cancer cachexia.” Surg. Oncol. 8.3 (1999): 133-41.
12. Bruera, E. “ABC of palliative care. Anorexia, cachexia, and nutrition.” BMJ 315.7117 (1997): 1219-22.
13. Fearon, K. C. and A. G. Moses. “Cancer cachexia.” Int. J. Cardiol. 85.1 (2002): 73-81.
14. Inui, A. “Cancer anorexia-cachexia syndrome: current issues in research and management.” CA Cancer J. Clin. 52.2 (2002): 72-91.
15. Kojima, M. et al. “Ghrelin: discovery of the natural endogenous ligand for the growth hormone secretagogue receptor.” Trends Endocrinol. Metab 12.3 (2001): 118-22.
16. Nakazato, M. et al. “A role for ghrelin in the central regulation of feeding.” Nature 409.6817 (2001): 194-98.
17. Smith, R. G. et al. “Peptidomimetic regulation of growth hormone secretion.” Endocr. Rev. 18.5 (1997): 621-45.
18. Wisse, B. E., M. W. Schwartz, and D. E. Cummings. “Melanocortin signaling and anorexia in chronic disease states.” Ann. N.Y. Acad. Sci. 994 (2003): 275-81.
19. Wren, A. M. et al. “Ghrelin enhances appetite and increases food intake in humans.” J. Clin. Endocrinol Metab 86.12 (2001): 5992.

1. Use of a secretagogue compound for the preparation of a medicament for the prophylaxis or treatment of cancer cachexia in an individual in need of such treatment.

2. The use according to claim 1, wherein the secretagogue is ghrelin or a pharmaceutially acceptable salt thereof.

3. The use according to claims i or 2, wherein the secretagogue is a ghrelin-like compound or a pharmaceutically acceptable salt thereof wherein the ghrelin-like compound comprises a structure defined by formula I
Z¹-(X¹)ₘ—(X²)—(X³)ₙ-Z², wherein
Z¹ is an optionally present protecting group
each X¹ is independently selected from an amino acid, wherein said amino acid is selected from naturally occurring and synthetic amino acids,
X² is any amino acid selected from naturally occurring and synthetic amino acids, said amino acid being modified with a bulky hydrophobic group, preferably an acyl group, or a fatty acid,
each X³ is independently selected from an amino acid, wherein said amino acid is selected from naturally occurring and synthetic amino acids,
wherein one or more of X¹ and X³ optionally may be modified with a bulky hydrophobic group, preferably an acyl group, or a fatty acid,
Z² is an optionally present protecting group,
m is an integer in the range of from 1-10
n is 0 or an integer in the range of from 1-35.

4. The use according to claim 3, wherein m is an integer in the range of from 1-9, such as of from 1-8, such as of from 1-7, such as of from 1-6, such as of from 1-5, such as of from 1-4, such as of from 1-3, such as of from 1-2,. such as 2.

5. The use according to any of claims 3 or 4, wherein X² is selected from the group of modified Ser, modified Cys and modified Lys, such as wherein X² is modified Ser.

6. The use according to any of claims 3 to 5, wherein the ghrelin-like compound is selected from a compound of
Z¹-Gly-(X¹)ₘ₋₁—(X²)—(X³)ₙ-Z², formula II
Z¹-Gly-Ser-(X²)—(X³)ₙ-Z², and formula III
Z¹-Gly-(X²)—(X³)ₙ-Z². formula IV

7. The use according to claim 6, wherein the ghrelin-like compound is having formula III.

8. The use according to any of claims 6 or 7, wherein (X³)ₙ comprises a sequence selected from one or more of the sequences shown below:
Phe Leu Ser Pro Glu His Gln
Phe Leu Ser Pro Glu His
Phe Leu Ser Pro Glu
Phe Leu Ser Pro
Phe Leu Ser
Phe Leu
Phe

9. The use according to any of claims 3 to 8, wherein n is an integer in the range of from 1-25, such as of from 1-24, such as from 1-15, such as of from 1-10, such as of from 10-25, such as of from 10-24, such as of from 15-25, such as of from 15-24.

10. The use according to any of claims 3 to 9, wherein (X³)ₙ is selected from one or more of the sequences shown below:
Phe Leu Ser Pro Glu His Gln Arg Val Gln Gln Arg
Lys Glu Ser Lys Lys Pro Pro
Ala Lys Leu Gln Pro Arg
Phe Leu Ser Pro Glu His Gln Arg Val Gln Gln Arg
Lys Glu Ser Lys Lys Pro Pro
Ala Lys Leu Gln Pro
Phe Leu Ser Pro Glu His Gln Arg Val Gln Gln Arg
Lys Glu Ser Lys Lys Pro Pro
Ala Lys Leu Gln
Phe Leu Ser Pro Glu His Gln Arg Val Gln Gln Arg
Lys Glu Ser Lys Lys Pro Pro
Ala Lys Leu
Phe Leu Ser Pro Glu His Gln Arg Val Gln Gln Arg
Lys Glu Ser Lys Lys Pro Pro
Ala Lys
Phe Leu Ser Pro Glu His Gln Arg Val Gln Gln Arg
Lys Glu Ser Lys Lys Pro Pro
Ala
Phe Leu Ser Pro Glu His Gln Arg Val Gln Gln Arg
Lys Glu Ser Lys Lys Pro Pro
Phe Leu Ser Pro Glu His Gln Arg Val Gln Gln Arg
Lys Glu Ser Lys Lys Pro
Phe Leu Ser Pro Glu His Gln Arg Val Gln Gln Arg
Lys Glu Ser Lys Lys
Phe Leu Ser Pro Glu His Gln Arg Val Gln Gln Arg
Lys Glu Ser Lys
Phe Leu Ser Pro Glu His Gln Arg Val Gln Gln Arg
Lys Glu Ser
Phe Leu Ser Pro Glu His Gln Arg Val Gln Gln Arg
Lys Glu
Phe Leu Ser Pro Glu His Gln Arg Val Gln Gln Arg
Lys
Phe Leu Ser Pro Glu His Gln Arg Val Gln Gln Arg
Phe Leu Ser Pro Glu His Gln Arg Val Gln Gln
Phe Leu Ser Pro Glu His Gln Arg Val Gln
Phe Leu Ser Pro Glu His Gln Arg Val
Phe Leu Ser Pro Glu His Gln Arg
Phe Leu Ser Pro Glu His Gln
Phe Leu Ser Pro Glu His
Phe Leu Ser Pro Glu
Phe Leu Ser Pro
Phe Leu Ser
Phe Leu
Phe

11. The use according to any of claims 3 to 10, wherein the acyl group is selected from a C1-C35 acyl group, such as a C1-C20 acyl group, such as a C1-C15 acyl group, such as a C6-C15 acyl group, such as a C6-C12 acyl group, such as a C8-C12 acyl group.

12. -The use according to any of claims 3 to 11, wherein the acyl group is selected from the group of C7 acyl group, C8 acyl group, C9 acyl group, C10 acyl group, C11 acyl group, and C12 acyl group.

13. The use according to any of claims 3 to 12, wherein the acyl group is selected from the group of C8 acyl group, and C10 acyl group.

14. The use according to any of claims 3 to 13, wherein the acyl group is selected from the group of C7 acyl group, C9 acyl group, and C11 acyl group, such as from the group of C9 acyl group and C11 acyl group.

15. The use according to any of preceding claims 1 to 14, wherein the medicament is in a formulation for subcutaneous administration.

16. The use according to any of claims 1 to 15, wherein the medicament formulation comprises the secretagogue or a pharmaceutically acceptable salt thereof.

17. The use according to any of claims 15 to 16, wherein the formulation comprises the secretagogue or a salt thereof as a lyophilisate and the formulation further comprises a solvent, said lyophilisate and said solvent being in separate compartements until administration.

18. The use according to any of claims 15 to 17, wherein the formulation is a solution of the secretagogue or a salt thereof.

19. The use according to claim 17 or 18, wherein the solvent is saline.

20. The use according to any of claims 1 to 19, wherein the medicament is administered prior to or during a meal.

21. The use according to any of claims 1 to 20, wherein the medicament is administered in a concentration equivalent to from 10 ng to 10 mg ghrelin per kg body-weight.

22. The use according to claim 21, wherein the medicament is administered in a concentration equivalent to from 0.1 μg to 1 mg ghrelin per kg bodyweight, such as from 0.5 μg to 0.5 mg ghrelin per kg bodyweight, such as from 1.0 μg to 0.1 mg ghrelin per kg bodyweight, such as from 1.0 μg to 50 μg ghrelin per kg bodyweight, such as from 1.0 μg to 10 μg ghrelin per kg bodyweight.

23. The use according to claim 22, wherein the medicament is administered in a concentration equivalent to from 0.1 μg to 1 mg ghrelin per kg bodyweight, such as from 0.5 μg to 0.5 mg ghrelin per kg bodyweight, such as from 1.0 μg to 0.1 mg ghrelin per kg bodyweight, such as from 1.0 μg to 50 μg ghrelin per kg bodyweight, such as from 1.0 μg to 10 μg ghrelin per kg bodyweight.

24. The use according to any of claims 1 to 23, wherein the medicament is administered as a bolus prior to or during a meal, said bolus comprising an amount of the ghrelin-like compound or a salt thereof equivalent to from 0.3 μg to 600 mg ghrelin

25. The use according to claim 24, wherein the medicament is administered as a bolus prior to or during a meal, said bolus comprising an amount of the ghrelin-like compound or a salt thereof equivalent to from 2.0 μg to 200 mg ghrelin, such as from 5.0 μg to 100 mg ghrelin, such as from 10 μg to 50 mg ghrelin, such as from 10 μg to 5 mg ghrelin, such as from 10 μg to 1.0 mg ghrelin.

26. The use according to any of claims I to 25, wherein the medicament is administered from one to three times daily, each administration being during a meal or at the most 180 minutes prior to a meal, such as at the most 90 minutes prior to a meal, e.g. at the most 45 minutes prior to a meal, such as at the most 30 minutes prior to a meal, such as at the most 25 minutes prior to a meal, such as at the most 20 minutes prior to a meal, such as at the most 15 minutes prior to a meal, such as at the most 10 minutes prior to a meal, such as at the most 5 minutes prior to a meal.

27. The use according to claim 26, wherein the medicament is administered three times daily.

28. The use according to any of claims 1 to 27, wherein the cancer cachexia is caused by a catabolic disorder.

29. The use according to any of claims 1 to 27, wherein the cancer cachexia is caused by an anorectic disorder.

30. The use according to any of claims 1 to 28, where the individual is suffering from a cancer selected from lung cancer, pancreatic cancer, liver cancer, and GI tract cancers.

31. Use according to any of claims 1 to 30, wherein said medicament is administered in combination with a chemotherapy medicament.

32. The use according to any of claims 1-31, wherein the treatment or prevention of cancer cachexia leads to stimulation of appetite, stimulation of food intake, stimulation of weight gain or weight maintenance, and/or increased body fat mass.

33. A method for preventing or treating cancer cachexia, comprising administerinig to an individual in need thereof an effective amount of a secretagogue as defined in any of claims 2-14.

34. A method for preventing or treating cancer, comprising administering to an individual in need thereof an effective amount of a secretagogue as defined in any of claims 2-14, in combination with an anti-neoplastic treatment.

35. The method according to claim 34, wherein the antineoplastic treatment is radiotherapy.

36. The method according to claim 34, wherein the antineoplastic treatment is chemotherapy.

37. The method according to any of claims 33-36, wherein the treatment or prevention of cancer cachexia leads to stimulation of appetite, stimulation of food intake, stimulation of weight gain or weight maintenance, and/or increased body fat mass.

38. Use of a ghrelin-like compound or a pharmaceutically acceptable salt thereof for the preparation of a medicament for prophylaxis or treatment of cachexia in an individual by administering a subcutaneous dosage of said medicarment to the individual,
wherein the ghrelin-like compound comprises a structure defined by formula I
Z¹-(X¹)ₘ—(X²)—(X³)ₙ-Z², wherein
Z¹ is an optionally present protecting group
each X¹ is independently selected from an amino acid, wherein said amino acid is selected from naturally occurring and synthetic amino acids,
X² is any amino acid selected from naturally occurring and synthetic amino acids, said amino acid being modified with a bulky hydrophobic group, preferably an acyl group, or a fatty acid,
each X³ is independently selected from an amino acid, wherein said amino acid is selected from naturally occurring and synthetic amino acids,
wherein one or more of X¹ and X³ optionally may be modified with a bulky hydrophobic group, preferably an acyl group, or a fatty acid,
Z² is an optionally present protecting group,
m is an integer in the range of from 1-10
n is 0 or an integer in the range of from 1-35.

39. Use according to claim 38, wherein the ghrelin-like compound is as defined in any of claims 4-14.

40. Use according to claim 38 or 39, wherein the medicament is in a formulation for subcutaneous administration.

41. Use according to claim 40, wherein the formulation comprises the ghrelin-like compound or a pharmaceutically acceptable salt thereof.

42. Use according to any of the preceding claims 40 or 41, wherein the formulation
comprises the ghrelin-like compound or a salt thereof as a lyophilisate and the formulation further comprises a solvent, said lyophilisate and said solvent being in separate compartements until administration.

43. Use according to any of the preceding claims 40 or 41, wherein the formulation is a solution of the ghrelin-like compound or a salt thereof.

44. Use according to claim 42 or 43, wherein the solvent is saline.

45. Use according to any of the preceding claims, wherein the medicament is administered as defined in any of claims 20-27.

46. Use according to any of claims 38 to 45, wherein the medicament is ad ministered from one to three times daily, each administration being prior to or during a meal, preferably less than 180 minutes prior to a meal, such as less than 90 minutes prior to a meal, for example less than 45 minutes prior to a meal, such as less than 30 minutes prior to a meal, for example less than 25 minutes prior to a meal, such as less than 20 minutes prior to a meal, for example less than 15 minutes prior to a meal, such as about 10 minutes prior to a meal, for example about 5 minutes prior to a meal, such as immediately prior to a meal, or during a meal, such as less than 90 minutes after commencing a meal, for example less than 45 minutes after commencing a meal, such as less than 30 minutes after commencing a meal, for example less than 25 minutes after commencing a meal, such as less than 20 minutes after commencing a meal, for example less than 15 minutes after commencing a meal, such as less than 10 minutes after commencing a meal, for-example less than 5 minutes after commencing a meal.

47. Use according to claim 46, wherein the medicament is administered from one to three times daily, preferably once prior to or during breakfast and/or once prior to or during lunch and/or once prior to or during dinner.

48. A ghrelin-like compound wherein the ghrelin-like compound is defined by formula I
Z¹-(X¹)ₘ—(X²)—(X³)ₙ-Z², wherein
Z¹ is an optionally present protecting group
each X¹ is independently selected from an amino acid, wherein said amino acid is selected from naturally occurring and synthetic amino acids,
X² is any amino acid selected from naturally occurring and synthetic amino acids, said amino acid being modified with an acyl group, wherein the acyl group is selected from the group of C7 acyl group, C9 acyl group, and C11 acyl group, such as from the group of C9 acyl group and C11 acyl group.
each X³ is independently selected from an amino acid, wherein said amino acid is selected-from naturally occurring and synthetic amino acids,
Z² is an optionally present protecting group,
wherein one or more of X¹ and X³ optionally may be modified by a bulky hydrophobic group, preferably an acyl group, or a fatty acid,
m is 0 or an integer in the range of from 1-10
n is 0 or an integer in the range of from 1-35.

49. The compound according to claim 48, wherein m is an integer in the range of from 1-9, such as of from 1-8, such as of from 1-7, such as of from 1-6, such as of from 1-5, such as of from 1-4, such as of from 1-3, such as of from 1-2, such as 2.

50. The compound according to any of claims 48 and 49, wherein X² is selected from the group of modified Ser, modified Cys and modified Lys, such as wherein X² is modified Ser.

51. The compound according to any of the claims 48 to 50, wherein the ghrelin-like compound is selected from a compound of
Z¹-Gly-(X¹)ₘ₋₁—(X²)—(X³)ₙ-Z², formula II
Z¹-Gly-Ser-(X²)—(X³)ₙ-Z², and formula III
Z¹-Gly-(X²)—(X³)ₙ-Z²; formula IV

52. The compound according to claim 51, wherein the ghrelin-like compound is having formula III.

53. The compound according to any of claims 48 to 52, wherein n is an integer in the range of from 1-25, such as of from 1-24, such as from 1-15, such as of from 1-10, such as of from 10-25, such as of from 10-24, such as of from 15-25, such as of from 15-24.

54. The compound according to any of claims 48 to 53, wherein (X³)ₙ comprises a sequence selected from one or more of the sequences shown below:
Phe Leu Ser Pro Glu His Gin
Phe Leu Ser Pro Glu His
Phe Leu Ser Pro Glu
Phe Leu Ser Pro
Phe Leu Ser
Phe Leu
Phe

55. The compound according to any of claims 48 to 54, wherein (X³)ₙ is selected from one or more of the sequences shown below.
Phe Leu Ser Pro Glu His Gln Arg Val Gln Gln Arg
Lys Glu Ser Lys Lys Pro Pro
Ala Lys Leu Gln Pro Arg
Phe Leu Ser Pro Glu His Gln Arg Val Gln Gln Arg
Lys Glu Ser Lys Lys Pro Pro
Ala Lys Leu Gln Pro
Phe Leu Ser Pro Glu His Gln Arg Val Gln Gln Arg
Lys Glu Ser Lys Lys Pro Pro
Ala Lys Leu Gln
Phe Leu Ser Pro Glu His Gln Arg Val Gln Gln Arg
Lys Glu Ser Lys Lys Pro Pro
Ala Lys Leu
Phe Leu Ser Pro Glu His Gln Arg Val Gln Gln Arg
Lys Glu Ser Lys Lys Pro Pro
Ala Lys
Phe Leu Ser Pro Glu His Gln Arg Val Gln Gln Arg
Lys Glu Ser Lys Lys Pro Pro
Ala
Phe Leu Ser Pro Glu His Gln Arg Val Gln Gln Arg
Lys Glu Ser Lys Lys Pro Pro
Phe Leu Ser Pro Glu His Gln Arg Val Gln Gln Arg
Lys Glu Ser Lys Lys Pro
Phe Leu Ser Pro Glu His Gln Arg Val Gln Gln Arg
Lys Glu Ser Lys Lys
Phe Leu Ser Pro Glu His Gln Arg Val Gln Gln Arg
Lys Glu Ser Lys
Phe Leu Ser Pro Glu His Gln Arg Val Gln Gln Arg
Lys Glu Ser
Phe Leu Ser Pro Glu His Gln Arg Val Gln Gln Arg
Lys Glu
Phe Leu Ser Pro Glu His Gln Arg Val Gln Gln Arg
Lys
Phe Leu Ser Pro Glu His Gln Arg Val Gln Gln Arg
Phe Leu Ser Pro Glu His Gln Arg Val Gln Gln
Phe Leu Ser Pro Glu His Gln Arg Val Gln
Phe Leu Ser Pro Glu His Gln Arg Val
Phe Leu Ser Pro Glu His Gln Arg
Phe Leu Ser Pro Glu His Gln
Phe Leu Ser Pro Glu His
Phe Leu Ser Pro Glu
Phe Leu Ser Pro
Phe Leu Ser
Phe Leu
Phe

56. A pharmaceutical composition comprising the ghrelin-like compound as defined in any of the claims 48 to 55 or a pharmaceutically acceptable salt thereof and pharmaceutically acceptable carriers, vehicles and/or excipients.

57. The pharmaceutical composition according to claim 56, wherein said composition further comprises transport molecules, such as liposomes, micelles, iscoms, and/or microspheres.

58. The pharmaceutical composition according to any of claims 56 to 57, comprising a secretagogue conjugate.

59. The pharmaceutical composition according to claim 58, wherein said conjugate is comprised of a secretagogue conjugated to one or more of:
a) a polymer molecule
b) an oligosaccharide moiety
c) the Fc region of an IgG

60. A medical packaging comprising one or more dosage units of the pharmaceutical composition as defined in any of claims 56 to 59.

61. The medical packaging according to claim 60, said packaging comprising from one to three dosage units.

62. The medical packaging according to claim 61, having three dosage units.

63. The medical packaging according to claim 60, said packaging having from 7 to 21 dosage units.

64. The medical packaging according to any of the claims 60 to 63, wherein said dosage unit comprises an amount of the ghrelin-like compound or a salt thereof equivalent to from 0.3 μg to 600 mg ghrelin, such as of from 2.0 μg to 200 mg ghrelin, such as from 5.0 μg, to 100 mg ghrelin, such as from 10 μg to 50 mg ghrelin, such as from 10 μg to 5 mg ghrelin, such as from 10 μg to 1.0 mg ghrelin.

65. The medical packaging according to any of the claims 60 to 64, comprising instructions for administering the pharmaceutical composition.

66. The medical packaging according to claim 65, wherein said instructions include instructions referring to administration of said pharmaceutical composition during a meal or at the most 180 minutes prior to a meal, such as at the most 90 minutes prior-to a meal, such as at the most 45 minutes prior to a meal, such as at most 30 minutes prior to a meal, such as at the most 25 minutes prior to a meal, such as at the most 20 minutes prior to a meal, such as at the most 15 minutes prior to a meal, such as at the most 10 minutes prior to a meal, such as at the most 5 minutes of a meal, such as immediately prior to a meal.

67. The medical packaging according to any of claims 60 to 66, wherein the packaging is in the form of a cartridge, such as a cartridge for an injection pen.

68. Use of the ghrelin-like compound as defined in any of claims 48 to 55, or a pharmaceutically acceptable salt thereof, for the preparation of a medicament for the treatment of an individual in need thereof.

69. The use according to claim 68, wherein said medicament is in a formulation for subcutaneous administration.

70. The use according to any of claims 68 or 69, wherein the medicament comprises the ghrelin-like compound or a salt thereof as a lyophilisate and the medicament further comprises a solvent, said lyophilisate and said solvent being in separate compartements until administration.

71. The use according to any of the preceding claims 68 to 70 wherein the medicament comprises a solution of the ghrelin-like compound or a salt thereof.

72. The use according to claim 70 or 71, wherein the solvent is saline.

73. The use according to any of claims 68 to 72, wherein the medicament is administered before a meal or during the intake of a meal as defined in any of claims 45-47.

74. A method for monitoring the effect of a treatment of an individual with a secretagogue, comprising measuring the blood level in said individual of IGF-1, IGFBP-3, and/or -ALS.

75. A method for preventing or treating cachexia, comprising administering to an individual in need thereof an effective amount of a secretagogue and an effective amount of a NSAID medicament.

76. The method according to claim 75, wherein the secretagogue is as defined in any of claims 2 to 14.

77. Use of a ghrelin-like compound or a pharmaceutically acceptable salt thereof for the preparation of a medicament for stimulation of appetite in an individual by administering a subcutaneous dosage of said medicament to the individual,
wherein the ghrelin-like compound comprises a structure defined by formula I
Z¹-(X¹)ₘ—(X²)—(X³)ₙ-Z², wherein
Z¹ is an optionally present protecting group
each X¹ is independently selected from an amino acid, wherein said amino acid is selected from naturally occurring and synthetic amino acids,
X² is any amino acid selected from naturally occurring and synthetic amino acids, said amino acid being modified with a bulky hydrophobic group, preferably an acyl group, or a fatty acid,
each X³ is independently selected from an amino acid, wherein said amino acid is selected from naturally occurring and synthetic amino acids,
wherein one or more of X¹ and X³ optionally may be modified with a bulky hydrophobic group, preferably an acyl group, or a fatty acid,
Z² is an optionally present protecting group,
m is an integer in the range of from 1-10
n is 0 or an integer in the range of from 1-35.

78. Use according to claim 77, wherein the ghrelin-like compound is as defined in any of claims 4-14.

79. Use according to claim 77 or 78, wherein the medicament is in a formulation for subcutaneous administration.

80. Use according to claim 79, wherein the formulation is as defined in any of claims 41 or 42.

81. Use according to claim 80, wherein the solvent is saline.

82. Use according to any of claims 77 to 81, wherein the medicament is administered as defined in any of claims 45-47.
